(19) United States
(12) Patent Application Publication
(10) Pub. No.: US 2024/0082366 A1
(43) Pub. Date: Mar. 14, 2024

(54) PROBIOTIC SULFATION OF SECONDARY BILE ACIDS

(71) Applicant: Danmarks Tekniske Universitet, Kongens Lyngby (DK)

(72) Inventors: Morten Otto Alexander Sommer, Kongens Lyngby (DK); Felipe Gonzalo Tueros Farfan, Kongens Lyngby (DK); Christian Bille Jendresen, Kongens Lyngby (DK); Alex Toftgaard Nielsen, Kongens Lyngby (DK); Karl Alex Hedin, Kongens Lyngby (DK)

(21) Appl. No.: 18/269,932

(22) PCT Filed: Jan. 25, 2022
(86) PCT No.: PCT/EP2022/051600

§ 371 Date: Jun. 27, 2023

(30) Foreign Application Priority Data
Jan. 26, 2021 (EP) 21153446.6

(51) Int. Cl. A61K 38/45; A61K 35/741; A61K 36/064; C12N 15/70; C12N 15/81

(52) U.S. Cl. CPC A61K 38/45; A61K 35/741; A61K 36/064; C12N 15/70; C12N 15/81; C12Y 208/02014; A61K 2035/115

(57) ABSTRACT

The invention relates to a microbiome-based therapeutic composition comprising an engineered probiotic cell expressing a sulfotransferase and the use of the microbiome-based therapeutic composition in medicine and a therapeutic regimen.

### FIELD

The invention is in the field of bile acid disorders and complications from pathologies that result in unbalanced bile acid pools. The invention relates to a therapeutic composition comprising a system for sulfating bioactive molecules in the gastrointestinal tract, wherein the system comprises engineered probiotica expressing a sulfotransferase. Specifically, the bioactive molecules are secondary bile acids such as deoxycholic acid (DCA) and lithocholic acid (LCA) and the probiotica is Escherichia coli such as E. coli Nissle 1917 (EcN) or Saccharomyces such as Saccharomyces boulardii. The composition can be used for regulation of unbalanced bile acid pools and for prevention or treatment of disorders associated with unbalanced bile acid pools. Such disorders include e.g. intestinal inflammation, cancer, hepatic inflammation, cirrhosis, and hepatic diseases. Thus, a composition of the invention can be used in the treatment of or in the avoidance or reduction in the development of carcinogenesis in the intestine and/or in the liver.

### BACKGROUND

Bile acids are key digestive molecules, that enable fat emulsification for appropriate absorption of fatty acids and liposoluble molecules through the intestine. Bile acids are the end product metabolism of cholesterol, they are synthetized in the liver from the contribution of at least 14 different enzymes. Primary bile acids, cholic acid (CA) and chenodeoxycholic acid (CDCA) are normally conjugated with glycine or taurine, forming bile salts. First microbial modification involves hydrolysing the amino acid moiety from the bile acid body by bile salt hydrolases (BSH) positive bacteria, gatekeeping further microbial bile acid modifications. Primary unconjugated bile acid leftovers are further metabolized by microorganisms in the large intestine producing secondary bile acids (SBA)s, deoxycholic acid (DCA) and lithocholic acid (LCA) by 7a-dehydroxylation of CA and CDCA, respectively. No active transport of bile acids takes place in the large intestine; however, secondary bile acids can translocate into the blood stream by passively diffusing through the enterocytes. The harmful effects of secondary bile acids in gut and peripheral organs exposed to these molecules, includes gallstones, inflammatory diseases, onset of hepatic, intestinal and colon cancer. This highlights the importance of gut microbiota in health and disease.

Cytosolic sulfotransferases (SULTs) are responsible for the sulfation of small molecules such as neurotransmitters, steroids, xenobiotics, and bile acids. This action decreases their bioactivity and increases the solubility of such molecules, channelling them to known excretion routes.

Endeavours have been made to successfully expressed active sulfotransferases in bacteria, in order to characterize the enzymes or produce sulfated products.

WO18195097 relates to compositions comprising populations of commensal bacteria isolated from a microbiome sample of a mammalian subject and engineered to express a heterologous polynucleotide, wherein commensal bacteria are adapted to colonize or configured for colonization of a mammal. It also relates to the use thereof for delivering a therapeutic polypeptide to a mammal, for example by administering the engineered commensal bacteria.

### SUMMARY

The object of the present invention is to provide a therapeutic composition comprising a system for sulfating bioactive molecules in the gastrointestinal tract, wherein the system comprises engineered probiotica expressing a sulfotransferase. Preferably, said sulfotransferase is of heterologous origin and more preferably, the sulfotransferase is the human sulfotransferase SULT2A1. Specifically, the bioactive molecules are the secondary bile acids such as lithocholic acid (LCA), deoxycholic acid (DCA), taurodeoxycholic acid (TDCA), glycodeoxycholic acid (GDCA), glycolithocholic acid (GLCA), ursodeoxycholic acid (UDCA), taurolithocholic acid (TLCA), tauroursodeoxycholic acid (TUDCA) and glycoursodeoxycholic acid (GUDCA). It is preferred that the secondary bile acids are deoxycholic acid (DCA) and lithocholic acid (LCA) and the probiotica is E. coli, such as E. coli Nissle 1917 (EcN) or Saccharomyces such as Saccharomyces boulardii. The therapeutic composition can be used for regulation of unbalanced bile acid pools and for prevention or treatment of disorders associated with unbalanced bile acid pools. Such disorders include those described herein. In particular a therapeutic composition of the invention can be used to avoid development of carcinogenesis both intestinal and in the liver.

A first aspect of the invention relates to a microbiome-based therapeutic composition comprising an engineered probiotic cell expressing a sulfotransferase. The expression of a sulfotransferase in a host may result in enhanced sulfation of, amongst other molecules, secondary bile acids. In an appropriate host cell, such as the probiotic cell E. coli including EcN or Saccharomyces such as S. boulardii, the expression of a sulfotransferase capable of sulfating secondary bile acids, can enhance the level of sulfation in the gastrointestinal tract. More specifically, the capable sulfotransferase is one that, upon expression in the host cell can enhance sulfation of DCA and LCA.

The invention also relates to the use of the microbiome-based therapeutic composition in medicine and a therapeutic regimen. The harmful effect of the secondary bile acids, especially DCA and LCA, in the gastrointestinal tract, accounts for primary disease induction and disease progression. Specifically, the therapeutic composition, which comprises the probiotic cell that expresses—under regulation of a constitutive promoter or inducible promoter—a sulfotransferase and, optionally a sulfate permease, in combination with e.g., other sulfate related genes, can be used in the treatment of disorders related to maladapted bile acid levels. Examples of diseases related to maladapted levels of bile acids, can be diseases such as but not limited to intestinal inflammation and/or cancer, hepatic inflammation, cirrhosis and/or hepatic diseases. The microbiome-based therapeutic regimen for modulating the concentrations of bile acids via sulfation, wherein a microbiome-based therapeutic composition is administered by oral or rectal administration. In the microbiome-based therapeutic regimen the dosage of the microbiome-based therapeutic may be determined on an individual basis, and the amount of the administrated microbiome-based therapeutic can be assessed and monitored on the basis of the individual patient's age, weight, food intake, macrobiotic flora and level of sulfated secondary bile acids, as measured from e.g., blood samples, urine samples and/or faecal samples.

### ABBREVIATIONS

PAPS 3′-phosphoadenosine 5′-phosphosulfate
DCA Deoxycholic acid
LCA Lithocholic acid
TDCA Taurodeoxycholic acid
GDCA Glycodeoxycholic acid
GLCA Glycolithocholic acid
UDCA Ursodeoxycholic acid
TLCA Taurolithocholic acid
TUDCA Tauroursodeoxycholic acid
GUDCA Glycoursodeoxycholic acid
GI Gastrointestinal
PCR Polymerase chain reaction
NAFLD Non-alcoholic fatty liver disease
NASH Nonalcoholic steatohepatitis
IBD Inflammatory bowel disease
CFU Colony-forming unit
MQ MilliQ
LC-MS/MS Liquid Chromatography with tandem mass spectrometry
YT Yeast Extract Tryptone
IPTG Isopropyl β-d-1-thiogalactopyranoside

### DEFINITIONS

As used herein the microbiome is to be understood as the gut microbiome, further defined as the microbial ensemble of species represented in the gastrointestinal (GI) tract and/or defined as the microorganisms, bacteria, viruses, protozoa, and fungi, and their collective genetic material present in the GI tract.

Microbiome-based therapeutic as described herein is defined as a therapeutic based on a microorganism which may or may not divide and grow in the gastrointestinal tract.

The terms “commensal bacteria” or “native bacteria” interchangeably refer to a bacterial cell or population of cells obtained from, and adapted to, or configured for the microbiome of a mammal. Commensal bacteria are adapted to colonize or configured for colonization in a mammal e.g. mucosal GI tract, mouth/pharynx, urogenital tract, skin, anus/rectum, cheek/mouth, or eye, and are not adapted for or configured for culture in a laboratory environment. Commensal bacteria are harvested from a mammal, then genetically transformed and finally reintroduced into the same or another mammal.

Commensal microorganisms provide the host with essential nutrients and metabolize indigestible compounds and in general contribute to the development of the intestinal architecture. Both host and commensal microorganisms have adapted to each other. Thus, a commensal microorganism is a microorganism that is adapted specifically to the host, through habituation in the intestines of the host. In general, a commensal microorganism is not suitable for culturing outside the host (Martin, R. et al., Role of commensal and probiotic bacteria in human health: a focus on inflammatory bowel disease, Microbial Cell Factories 2013).

Native/commensal bacteria that are adapted to specific hosts and are isolated therefrom are described in WO2018195097. Microorganisms described in WO2018195097 are not suitable for laboratory culturing and are cultured for less than 30 days following isolation from the host.

In that regard, a probiotic microorganism of the present invention is a microorganism that is not adapted to the specific host and is a microorganism that is suitable for culturing outside the host organism. Thus, a probiotic microorganism in the context of the present invention is not a commensal microorganism.

The terms “protein” and “polypeptide” are herein used interchangeably.

The term “probiotics” as used herein means a laboratory strain of a bacterial strain; a probiotic cell is not integrated into the microbiome of the host in a permanent way but is excreted from the host over a period of time. An example of such a probiotic strain is E. coli Nissle 1917. In terms of the invention, a probiotic is to be understood as live microorganisms which when administered in adequate amounts confer a health benefit on the host.

A “sulfotransferase” is a polypeptide capable of catalyzing the transfer of a sulfo or sulfate group from a donor molecule to an acceptor alcohol or amine functional group. More specific the sulfotransferase of the invention is a sulfotransferase that catalyzes the transfer of a sulfate onto a hydroxyl group. Herein, the definition of “sulfation” is the reaction wherein a sulfate is transferred from a donating molecule onto a hydroxyl moiety of an acceptor creating an organosulfate or sulfate-ester moiety. In the literature the terms “sulfation” and “sulfonation” have often be used interchangeably. Sulfation leads to a sulfate-ester moiety, i.e. a coupling of a sulfate group to an oxygen atom, whereas sulfonation couples a sulfate group directly to a carbon atom.

The term “sulfation” as used herein relates to the addition of a sulfate group to a hydroxy group through a sulfotransferase mediated reaction, such as but not limited to the addition of a sulfate group to a secondary bile acid in a relevant position of the molecule such as e.g. in the 3-position of a cholic acid, or addition of a sulfate to the 3- or 4-position of a catecholamine. A sulfotransferase may also add a sulfate group to other substances residing in the gastrointestinal tract.

A “genetically engineered microbial cell” is understood as a bacterial cell which has been transformed, engineered or is capable of transformation or engineering by a heterologous and/or recombinant polynucleotide sequence. The genetically engineered microbial cell is preferably a prokaryotic cell. Appropriate microbial cells include yeast cells, bacterial cells, archaebacterial cells, algae cells, and fungal cells.

A bacterial host cell or a genetically engineered microbial cell may be a probiotic cell derived from an E. coli strain, a Lactobacillus species, a Corynebacterium strain, a Bacillus strain, a Lactobacillus strain, a Streptococcus strain, an Enterococcus strain, a Lactococcus strain, or a Clostridium strain.

A capable host cell or a genetically engineered host cell may be a eukaryotic cell derived from a Saccharomyces cerevisiae strain, such as Saccharomyces boulardii, a Schizosaccharomyces strain such as Schizosaccharomyces pombe, a Pichia strain such as Pichia pastoris, a Kluveromyces strain such as Kluveromyces lactis or Kluveromyces marxianus or from a Lactobacillus strain such as, Lactobacillus acidophilus, Lactobacillus rhamnosus or Lactobacillus johnsonii.

Non-limiting examples of well-known probiotica are Bifidobacterium sp., Escherichia coli Nissle 1917 and the yeast Saccharomyces boulardii.

Probiotic microorganisms are often live microorganisms, which when administered to a host organism confer a health benefit on the host (Martin, R. et al., Role of commensal and probiotic bacteria in human health: a focus on inflammatory bowel disease, Microbial Cell Factories 2013).

In the context of the present invention, a probiotic is to be understood as live microorganisms which when administered in adequate amounts confer a health benefit on the host.

Thus, in embodiments of the present invention, the engineered probiotic cell of the present invention is a prokaryote such as a bacterium or yeast. In another embodiment, the engineered probiotic cell of the present invention is a gram-negative bacterium. In another embodiment, the engineered probiotic cell of the present invention is yeast. In yet another embodiment, the engineered probiotic cell of the present invention is a bacterial species selected from Roseburia spp., Eubacterium spp., Akkermansia spp., Christensensella spp., Propionibacterium spp., and Faecalibacterium spp, Lactobacillus spp., Bifidobacterium Spp., Streptococcus spp., and Escherichia spp.

In a preferred embodiment, the bacterial species is selected from Escherichia spp., such as but not limited to E. coli Nissle 1917. In a further preferred embodiment, the engineered probiotic cell of the present invention is E. coli Nissle 1917.

In other embodiments, the engineered probiotic cell of the present invention is a fungus. In a preferred embodiment the engineered probiotic cell of the present invention is a yeast preferably, a Saccharomyces cerevisiae strain, such as Saccharomyces boulardii, a Schizosaccharomyces strain such as Schizosaccharomyces pombe, a Pichia strain such as Pichia pastoris, a Kluveromyces strain such as Kluveromyces lactis or Kluveromyces marxianus or from a Lactobacillus strain such as, Lactobacillus acidophilus, Lactobacillus rhamnosus or Lactobacillus johnsonii.

A person skilled in the art will be aware of further microbiological strains when reading the present disclosure.

As used herein, the terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary amino acid sequence thereof.

As used herein, “codon optimization” refers to the process of optimizing the DNA sequence of a gene towards a specific host cell in order to improve expression of a gene of interest and increase the translational efficiency of a gene of interest by accommodating codon bias of the host organism. An example could be optimizing a human gene for expression in E. coli.

The term “functional gene” as used herein, refers to a nucleic acid molecule comprising a nucleotide sequence which encodes a protein or polypeptide, and which also contains regulatory sequences operably linked to said protein-coding nucleotide sequence such that the nucleotide sequence which encodes the protein or polypeptide can be expressed in/by the microbial cell bearing said functional gene. Thus, when cultivated at conditions that are permissive for the expression of the functional gene, said functional gene is expressed, and the microbial cell expressing said functional gene typically comprises the protein or polypeptide that is encoded by the protein coding region of the functional gene.

The term “overexpression” or “overexpressed” as used herein refers to a level of enzyme, protein or polypeptide expression that is greater than what is measured in a wild-type cell of the same species as the host cell that has not been genetically engineered.

The term “operably linked” as used herein, shall mean a functional linkage between a nucleic acid expression control sequence (such as a promoter, signal sequence, or array of transcription factor binding sites) and a second nucleic acid sequence, wherein the expression control sequence affects transcription and/or translation of the nucleic acid corresponding to the second sequence. Accordingly, the term “promoter” designates DNA sequences which usually “precede” a gene in a DNA polymer and provide a site for initiation of the transcription into mRNA. “Regulator” DNA sequences, also usually “upstream” of (i.e., preceding) a gene in a given DNA polymer, bind proteins that determine the frequency (or rate) of transcriptional initiation. Collectively referred to as “promoter/regulator” or “control” DNA sequence, these sequences which precede a selected gene (or series of genes) in a functional DNA polymer cooperate to determine whether the transcription (and eventual expression) of a gene will occur. DNA sequences which “follow” a gene in a DNA polymer and provide a signal for termination of the transcription into mRNA are referred to as transcription “terminator” sequences.

A bacterial host cell may further comprise control sequences enabling the controlled overexpression of endogenous or recombinant nucleic acid sequences. As defined above, the term “control sequence” which herein is synonymously used with the expression “nucleic acid expression control sequence”, comprises promoter sequences, signal sequence, or array of transcription factor binding sites, which sequences affect transcription and/or translation of a nucleic acid sequence operably linked to the control sequences.

A nucleic acid sequence may be placed under the control of an inducible promoter, which is a promoter that directs expression of a gene where the level of expression is alterable by environmental or developmental factors such as, for example, temperature, pH, anaerobic or aerobic conditions, light, transcription factors, bile acids and chemicals. Such promoters are referred to herein as “inducible” promoters, which allow one to control the timing of expression of the proteins used in the present invention. For E. coli and other bacterial host cells, inducible promoters are known to those of skill in the art. For S. boulardii and other yeast host cells, inducible promoters are known to those of skill in the art.

The term “genetically engineered” as used herein refers to the modification of the microbial cell's genetic make-up using molecular biological methods. The modification of the microbial cell's genetic make-up may include the transfer of genes within and/or across species boundaries, inserting, deleting, replacing and/or modifying nucleotides, triplets, genes, open reading frames, promoters, enhancers, terminators and other nucleotide sequences mediating and/or controlling gene expression. The modification of the microbial cell's genetic make-up aims to generate a genetically modified organism possessing particular, desired properties. Genetically engineered microbial cells can contain one or more genes that are not present in the native (not genetically engineered) form of the cell. Techniques for introducing exogenous nucleic acid molecules and/or inserting exogenous nucleic acid molecules (recombinant, heterologous) into a cell's hereditary information for inserting, deleting or altering the nucleotide sequence of a cell's genetic information are known to the skilled artisan. Genetically engineered microbial cells can contain one or more genes that are present in the native form of the cell, wherein said genes are modified and re-introduced into the microbial cell by artificial means. The term “genetically engineered” also encompasses microbial cells that contain a nucleic acid molecule being endogenous to the cell, and that has been modified without removing the nucleic acid molecule from the cell. Such modifications include those obtained by gene replacement, site-specific mutations, and related techniques.

The art is rich in patent and literature publications relating to “recombinant DNA” methodologies for the isolation, synthesis, purification and amplification of genetic materials for use in the transformation of selected host organisms. Thus, it is common knowledge to transform host organisms with “hybrid” viral or circular plasmid DNA which includes selected exogenous (i.e., foreign or “heterologous”) DNA sequences, in some cases recombined with native DNA sequences or additional exogenous DNA sequences, making up a recombinant DNA sequence. The procedures known in the art first involve generation of a transformation vector by enzymatically cleaving circular viral or plasmid DNA to form linear DNA strands. Selected foreign DNA strands usually including sequences coding for desired protein product are prepared in linear form through use of the same/similar enzymes. The linear viral or plasmid DNA is incubated with the foreign DNA in the presence of ligating enzymes capable of effecting a restoration process and “hybrid” vectors are formed which include the selected exogenous DNA segment “spliced” into the viral or circular DNA plasmid.

The term “nucleotide sequence encoding” generally refers to any polyribonucleotide or polydeoxyribonucleotide, which may be unmodified RNA or DNA or modified RNA or DNA, and generally represents the portion of a gene which encodes a certain polypeptide or protein. The term includes, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

Within the scope of the present invention, also nucleic acid/polynucleotide and polypeptide polymorphic variants, alleles, mutants, and interspecies homologs are comprised by those terms, that have an amino acid or nucleotide sequence that has greater than about 60% amino acid or nucleotide sequence identity, 65%, 70%, 75%, 80%, 85%, 90%, preferably 91%, 92%, 93%, 94%, 95%, 96%, 97%, 98% or 99% or greater amino acid or nucleotide sequence identity, preferably over a region of at least about 25, 50, 100, 200, 500, 1000, or more amino acids or nucleotides, to a polypeptide encoded by a wildtype protein or an endogenous nucleotide sequence. The term “sequence identity of [a certain] %” in the context of two or more nucleic acid or amino acid sequences means that the two or more sequences have nucleotides or amino acid residues in common in the given percent when compared and aligned for maximum correspondence over a comparison window or designated sequences of nucleic acids or amino acids (i.e. the sequences have at least 90 percent (%) identity). Percent identity of nucleic acid or amino acid sequences can be measured using a BLAST 2.0 sequence comparison algorithm with default parameters, or by manual alignment and visual inspection (see e.g. http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the invention. Software for performing BLAST analyses is publicly available through the National Center for Biotechnology Information (http://www.ncbi.nlm.nih.gov/). Examples of commonly used sequence alignment algorithms are
CLUSTAL Omega (http://www.ebi.ac.uk/Tools/msa/clustalo/),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B. The term “comprising of” also includes the term “consisting of”.

### DETAILED DESCRIPTION

Unless specifically defined, all technical and scientific terms used herein have the same meaning as commonly understood by a skilled person in the field of biochemistry, genetics, biology and molecular biology.

### A Microbiome-Based Therapeutic Composition

The present invention provides a microbiome-based therapeutic composition comprising an engineered probiotic cell expressing a sulfotransferase. A microbiome-based therapeutic is defined as a therapeutic composition comprising a genetically modified microorganism. The microbiome-based therapeutic of the invention, promotes a transient occupation of the host, wherein the microbiome-based therapeutic of the present invention is excreted through the faeces. Thus, the microbiome-based therapeutic of the present invention is not incorporated permanently into the endogenous microbiome of a host organism. In that regard, the microorganism-based therapeutic of the present invention is a therapeutic based on an engineered probiotic cell. The probiotic cell may be any microorganisms that, when administered in adequate amounts, confer a health benefit on the host, examples of such are members of the Lactobacillus species, members of the Bifidobacterium species, Streptococcus thermophilus, Escherichia coli (E. coli), Bacillus cereus, Clostridum butyricum, Enterococcus faecalis, Enterococcus faecium, Saccharomyces boulardii, Saccharaomyces cerevisiae and strains derived thereof.

Several probiotic strains are used in production of dairy products and dietary supplements, such as Lactobacillus bulgaricus and Streptococcus thermophilus, but also non-pathogenic strains of E. coli have gained impasse in the production of probiotic dietary supplements and in prophylactic treatment and in treatment of disease, the most well studied example of a probiotic E. coli strain is E. coli. Nissle 1917. Thus, in one aspect of the invention the probiotic cell is E. coli. In a preferred aspect of the invention the probiotic cell is E. coli Nissle 1917. In another aspect of the invention the probiotic cell is Saccharaomyces cerevisiae. In a preferred aspect of the present invention the probiotic cell is Saccharomyces boulardii.

The sulfotransferase expressed by the engineered probiotic cell is a polypeptide capable of transferring a sulfate from a donor molecule, such as 3′-phosphoadenosine 5′-phosphosulfate PAPS), onto an acceptor molecule such as deoxycholic acid (DCA) or lithocholic acid (LCA), such as the sulfotransferases mentioned in table 1. The sulfotransferase expressed may be any polypeptide having sulfotransferase activity directed to sulfation of secondary bile acids and xenobiotics, furthermore it may be a sulfotransferase selected from table 1.

TABLE 1
Sulfotransferases of the present invention

| | | Sulfotransferase | | SEQ ID |
|---|---|---|---|---|
| Enzyme | Source | ID | Reference* | NO: |
| SULT1A1 | Rattus | pst-5 | 1 | 1, 30 |
| | norvegicus | | | |
| SULT1ST1 | Danio rerio | pst-29 | 1 | 10, 35 |
| SULT6B1 | Danio rerio | pst-31 | 1 | 11 |
| SULT1A1 | Homo sapiens | pst-4 | 1 | 7, 31 |
| SULT1A1 - | Rattus | pst-26 | 1 | 2 |
| clone 2 | norvergicus | | | |
| dmST1 | Drosophila | pst-6 | 1, 2 | 12, 36 |
| | melanogaster | | | |
| dmST1 - | Drosophila | pst-27 | 1, 2 | 13, 37 |
| clone2 | melanogaster | | | |
| dmST3 - | Drosophila | pst-8 | 1, 2 | |
| variant A | melanogaster | | | |
| dmST4 | Drosophila | pst-10 | 1, 2 | |
| | melanogaster | | | |
| SULT1A1 | Equus ferus | pst-11 | 1 | 6, 34 |
| | caballus | | | |
| SULT1E1 | Gallus gallus | pst-12 | 1 | 14, 38 |
| | domesticus | | | |
| SULT1A1 | Canis lupus | pst-13 | 1 | 4, 32 |
| | familiaris | | | |
| SULT1A1 | Sus scrofa | pst-14 | 1 | 5, 33 |
| | domesticus | | | |
| SULT1B1 - | Gallus gallus | pst-15var | 1 | |
| clone1 | domesticus | (CBJ1146) | | |
| SULT1B1 - | Gallus gallus | pst-15var | 1 | |
| clone2 | domesticus | (CBJ1147) | | |
| SULT1B1 - | Gallus gallus | pst-15var | 1 | |
| clone3 | domesticus | (CBJ1148) | | |
| SULT1B1- | Gallus gallus | pst-16var | 1 | 15, 39 |
| predicted - | domesticus | (CBJ1149) | | |
| clone1 | | | | |
| SULT1B1- | Gallus gallus | pst-16var | 1 | 16, 40 |
| predicted - | domesticus | (CBJ1150) | | |
| clone2 | | | | |
| SULT1C1 | Gallus gallus | pst-17var | 1 | 17, 41 |
| | domesticus | (CBJ1151) | | |
| SULT2B1- | Gallus gallus | pst-18var | 1 | |
| variant1 | domesticus | (CBJ1152) | | |
| SULT2B1- | Gallus gallus | pst-18var | 1 | |
| predicted | domesticus | (CBJ1153) | | |
| SULT2B1- | Gallus gallus | pst-18var | 1 | |
| variant2 | domesticus | (CBJ1154) | | |
| SULT1A1 | Rattus | pst-25 | 1 | 3, 30** |
| (codon-opt) | norvegicus | | | |
| SSU-1 | Caenorhabditis | pst-28 | 1 | |
| | elegans | | | |
| SULT4A1 | Danio rerio | pst-30 | 1 | |
| AtSOT12 | Arabidopsis | pst-32 | 1 | |
| | Thaliana | | | |
| Cpz8 | Streptomyces | pst-33 | 1 | |
| LipE | Streptomyces | pst-34 | 1 | |
| SPPG_07427 | Sphenodon | pst-35 | 1 | |
| | punctatus | | | |
| | DAOM BR117 | | | |
| Hoch_6098 | Haliangium | pst-36 | 1 | |
| | ochraceum | | | |
| | DSM 14365 | | | |
| RradSPS_0172 | Rubrobacter | pst-37 | 1 | |
| | radiotolerans | | | |
| KMZ76263.1 | Zostera marina | pst-38 | 1 | |
| KMZ64288.1 | Zostera marina | pst-39 | 1 | |
| KMZ76264.1 | Zostera marina | pst-40 | 1 | |
| KMZ59959.1 | Zostera marina | pst-41 | 1 | |
| KMZ74024.1 | Zostera marina | pst-42 | 1 | 18, 42 |
| KMZ64284.1 | Zostera marina | pst-43 | 1 | |
| KMZ72298.1 | Zostera marina | pst-44 | 1 | |
| KMZ72292.1 | Zostera marina | pst-45 | 1 | |
| KMZ72296.1 | Zostera marina | pst-46 | 1 | |
| KMZ76265.1 | Zostera marina | pst-47 | 1 | |
| KMZ73756.1 | Zostera marina | pst-48 | 1 | 19, 43 |
| KMZ69186.1 | Zostera marina | pst-49 | 1 | |
| SULT2A1 | Homo sapiens | pst-50 | | 8, 29** |
| SULT2A1 | Homo sapiens | pst-51 | | 9, 29** |
| (codon-opt) | | | | |

*Reference 1: Jendresen, C. B., & Nielsen, A. T. (2019). Production of zosteric acid and other sulfated phenolic biochemicals in microbial cell factories. Nature Communications, 10. https://doi.org/10.1038/s41467-019-12022-x
*Reference 2: Hattori, K., Motohashi, N., Kobayashi, I., Tohya, T., Oikawa, M., & Tamura, H. O. (2008). Cloning, expression, and characterization of cytosolic sulfotransferase isozymes from Drosophila melanogaster. Bioscience, biotechnology, and biochemistry, 72(2), 540-547. https://doi.org/10.1271/bbb.70647
**Codon optimized variants encodes identical protein sequences to non-codon optimized variants.

The inventors found that a suitable sulfotransferase according to the invention is a sulfotransferase that is particularly suitable for sulfation secondary bile acids such as LCA and DCA, as exemplified in example 1. Other substrates for the sulfotransferase are for example further secondary bile acids, steroids, catecholamines such as dopamine and nor-adrenaline, serotonin, iodothyronines, eicosanoids, retinol, 6-hydroxymelatonin, ascorbate and vitamin D. The sulfotransferase substrates can be produced either endogenously or by foreign organisms, making these xenobiotic substances (xenobiotics). A xenobiotic is to be understood as any chemical compound that are of foreign origin or produced in a foreign organism different from the human organism. Examples are e.g. catecholamines produced by commensal bacteria or probiotics; such catecholamines are regarded as xenobiotics as there are of foreign origin.

The engineered probiotic cell may express a human sulfotransferase. As seen from the Examples herein a suitable engineered probiotic cell expresses a human sulfotransferase, wherein the sulfotransferase is human SULT2A1, as shown in SEQ ID NO: 29. In that regard, the present invention in exemplified embodiments relates to a microbiome-based therapeutic composition comprising an engineered probiotic cell expressing a sulfotransferase, wherein the sulfotransferase is human SULT2A1, as shown in SEQ ID NO: 29, or a functional homologue thereof, having at least 80% sequence identity to SEQ ID NO: 29.

In one aspect of the invention, the nucleic acid sequence according to any one of SEQ ID NOs: 1-19 is contained in the host cell or in nucleic acid construct. In a further aspect of the invention, the probiotic cell expresses one or more proteins of SEQ ID NOs: 29-43. In another aspect of the invention the one or more proteins expressed in the probiotic cell is selected from the group; SULT1A1, SULT1A1-clone 2 or a codon optimized SULT1A1 from R. norvegicus, SULT1A1, SULT1A1 from C. lupus familiaris, SULT1A1 from S. scrofa domesticus, SULT1A1 from Equus ferus caballus, SULT2A1 and codon optimized SULT2A1 from H. Sapiens.

In one aspect of the invention the host cell or the nucleic acid construct comprises one or more of the genes of SEQ ID NOs: 20-28.

The level of sulfation of bile acids is determined by a number of factors and cellular pathways, such as the sulfate assimilation pathway of E. coli. In this pathway sulfates are taken up into the cell by sulfate transport systems and associated proteins. Secondly, the sulfates are converted into 5′-adenylylsulfate (APS), and further to 3′-phospho-adenylylsulfate (PAPS) and sulfite, by specific enzymes. PAPS is enzymatically converted into 3′-phospho-adenylyl (PAP) and sulfite. Sulfite can be reduced to sulphide which can attach to O-acetylserine to synthesize cysteine from serine, in that way forming the basis for the biosynthetic cysteine production pathway. Thus, modifications to selected steps of the sulfate assimilation pathway can optimally enhance the sulfation of secondary bile acids, through modulation of the availability of sulfates in the cell. Examples of such modifications are presented in examples 3, 4 and 5. Example 3 presents an enhanced sulfation obtained by strengthening the cellular sulfate uptake by incorporation of a heterologous sulfate permease into E coli nissle 1917. Examples 4 and 5 show that knockout of specific sulfate assimilation pathway related proteins benefit the sulfation of secondary bile acids.

Examples of such sulfate assimilation pathway related proteins are ydeN (https://biocyc.org/gene?orgid=ECOLI&id=G6788), cysH (https://biocyc.org/gene?orgid=ECOLI&id=EG10189) and cysQ (https://biocyc.org/gene?orgid=ECOLI&id=EG10043).

In one aspect of the invention the probiotic cell expresses one or more of the proteins selected from the group Sbp, cysZ, cysP, cysU, cysW, cysA, cysD, cysN, cysC and cysQ from E. coli, and/or CysP from Bacillus subtilis. In one aspect of the invention the probiotic cell expresses one or more of the proteins selected from the group cysP, cysU, cysW, cysA, cysD, cysN, cysC and cysQ from E. coli, and/or CysP from Bacillus subtilis, encoded by SEQ ID NOs: 20-28.

In one embodiment of the invention the probiotic cell expresses one or more of the proteins selected from the group consisting of Sbp, cysZ, cysP, cysU, cysW, cysA, cysD, cysN, cysC from E. coli, and CysP from Bacillus subtilis.

In one aspect of the invention, one or more of the nucleic acid sequence(s) of SEQ ID NOs: 1-19, is/are contained in the host cell or in nucleic acid construct, encoding one more of the proteins according to any one of the amino acid sequence(s) of SEQ ID NOs: 29-43 or one or more functional homologues thereof, which amino acid sequences is at least 80% sequence identity to any one of SEQ ID NOs: 29-43, such as 90%, such as 95% sequence identity. In a further aspect the probiotic cell or the nucleic acid construct contains one or more of the nucleic acid sequence(s) of SEQ ID NOs: 20-28, encoding one more of the proteins according to any one of the amino acid sequence(s) of SEQ ID NOs: 44-52 or one or more functional homologues thereof, which amino acid sequences is at least 80% identical to any one of SEQ ID NOs: 44-52 such as 90%, such as 95% or such as 99% sequence identity

By the term “functional homolog” in the present context is meant a protein that has an amino acid sequence that is more than 80%, such as 85% or more, 90% or more, 95% or more, 96% or more, 97% or more, 98% or more, 99% or more or 100% identical to any one of SEQ ID NOs: 29-52 and has a function that is beneficial to achieve at least one advantageous effect of the invention, e.g. an increase of the total formation of sulfated secondary bile. In one embodiment a functional homologue is a protein which has an amino acid sequence that is more than 80%, such as 85% or more, 90% or more, 95% or more, 96% or more, 97% or more, 98% or more, 99% or more or 100% identical to any one of SEQ ID NOs: 29-52 and a functionality of more than 50%, such as 85%-100% or above 100% of any one of SEQ ID NOs: 29-52.

A nucleic acid construct according to the invention may comprise a nucleic acid sequence having at least 80% identity with any one of SEQ ID NOs: 1-19, 64 and/or any one of SEQ ID NOs: 20-28 operably linked to one or more promoter sequences that direct the expression of the coding sequence in probiotic cell.

The nucleic acid sequence may be manipulated in a variety of ways to provide for expression of any one of the proteins of SEQ ID NOs: 29-43 and/or 44-52 or functional variants thereof. Manipulation prior to insertion of the gene or genes into a plasmid may be desirable or necessary depending on the plasmid. The techniques for modifying nucleic acid sequences utilizing recombinant DNA methods are well known in the art.

The expression of a heterologous and homologous gene in a host cell, such as a probiotic cell according to invention, relies on the DNA sequence of the gene to be transcribed and translated. A gene native to mammalians might not be well expressed in a probiotic cell, since some codons commonly used by the mammalian expression system, are rarely used in the probiotics of this invention. Thus, optimization of the genetic sequence, in this disclosure referred to as codon optimization, can be done, when transferring a genetic sequence from one host to another host. In relation to the present invention the term codon optimized relates to optimization of the DNA or RNA sequence carrying the gene in question, wherein the encoding base codon of the gene is optimized towards the expression in the host cell, in terms of host cell codon frequency and/or usage, wherein host cell refer, in relation to the invention, to the probiotic cell. The inventors found in example 2 that codon optimization of the human SULT2A1 resulted in enhanced sulfation of secondary bile acids when operably linked to a strong constitutive promoter. Therefore, an engineered probiotic cell may express a human sulfotransferase, wherein SULT2A1 is codon optimized for expression in the probiotic cell. In particular, the engineered probiotic cell expresses a human sulfotransferase, wherein SULT2A1 is codon optimized for expression in E. coli as shown in SEQ ID NO: 9 or Saccharomyces as shown in SEQ ID NO: 64.

Thus, in preferred embodiments, the engineered probiotic cell comprises a nucleic acid sequence encoding the human sulfotransferase, wherein the nucleic acid sequence comprises SEQ ID NO: 9 or SEQ ID NO: 64, or the reverse complement thereof, or a functional homologues thereof with a nucleic acid sequence having at least 80% sequence identity, such as at least 90%, such as at least 95%, such as at least 99% or such as 100% sequence identity to any one of SEQ ID NO: 9 or SEQ ID NO: 64.

The expression of a sulfotransferase may be obtained by transformation of said probiotic cell with a plasmid. In the present context, a plasmid refers to a circular DNA encoding the gene or genes of interest, the plasmid can be of endogenous origin, of exogeneous origin and/or of synthetic origin, or a mixture of these. The plasmid may carry genes originating from the host cell or genes from other species. Expression of SULT2A1 can be obtained by transformation of said probiotic cell with a plasmid. In one or more embodiments, the expression of SULT2A1 is obtained by transformation of said probiotic cell with a pMUT plasmid.

Transformation is any common method for introducing a series of nucleic acids into the probiotic cell, such as but not limited to the method comprising chemically based transformation, lipid-based transformation, physically based transformation, bacteriophage transduction, conjugation.

Chemical transformation relates to the use of chemicals such as calcium phosphate or diethylaminoethyl-dextran. Lipid based transformation relates to the use of lipids such as cationinc lipids, zwitterioninc lipids, non-ioninc lipids. Physically based transformation methods could include methods such as microinjection, electroporation, heat shock, or passive integration. Bacteriophage transduction is to be understood as the transfer of genetic material into the probiotic cell using any bacteriophage based method. Conjugation is to be understood as the transfer of genetic material through cell-to-cell contact.

As shown in example 1, the inventors found that the expression of a sulfotransferase in different strains of E. coli resulted in sulfonation of the secondary bile acids, LCA and DCA. The inventors found that especially the human SULT2A1 gene resulted in enhanced sulfation of secondary bile acids upon transformation with a plasmid encoding the human SULT2A1. The transformation can also be done using electroporation (example 2). Thus, expression of SULT2A1 may be obtained by electroporation of said probiotic cell with a plasmid.

### Metabolic Pathway Engineering

Sulfation of secondary bile acids in a probiotic cell of the present invention is influenced by a number of different pathways and will be affected by import and export of sulfates, by permeases, and by sulfatases and sulfate related enzymes, adaptor proteins and substrate availability, as described above. Thus, engineering of theses pathways may affect the overall sulfation level obtained by the probiotic cell of the present invention. Examples of engineering of such pathways is provided in examples 3, 4, 5 and 6. Example 3 shows that the overall sulfation can be enhanced by expression of a heterologous sulfate transporter. Examples 5 and 6 describes how knockout (KO) of specific genes involved in different aspects of the sulfation is used to enhance the sulfation of the major bile acids LCA and DCA.

The endogenous sulfation of secondary bile acids and xenobiotics in the probiotic cell varies, but the sulfation relies on the activity of sulfotransferases as well as, e.g., sulfate permeases, sulfatases and sulfate related enzymes, adaptor proteins and substrate availability. Examples of sulfate permeases are, e.g., members of the sulfate permease family (SulP) which is represented in archaea, bacteria, fungi, plants and animals. Examples of bacterial sulfate permeases are cysZ from E. coli and cysP from Bacillus subtilis, the sulfate transport genes cysP, cysU, cysW and cysA from E. coli. Other examples which might influence the level of sulfation according to the invention through modulation of the sulfate uptake, maturation or recycling are members of the oxyanion transporter family.

In one aspect of the invention the expression of the sulfotransferase is encoded in a genetic construct, that can be inserted into a plasmid or chromosome.

In one aspect of the invention the nucleic acid sequence encoding a sulfotransferase of the present invention is incorporated into a pMUT plasmid. The pMUT plasmid is a plasmid derived from the endogenous E. coli pMUT plasmid, and it may be modified to contain the endogenous E. coli sulfate recycling genes, such as cysD, cysN, cysC and cysQ, as suggested in in FIG. 3A. As seen from the examples herein, the expression of a sulfotransferase can be obtained by transformation of the probiotic cell with a pMUT plasmid. Thus, it is preferred that the expression of a codon optimized human SULT2A1 is obtained by transformation of the probiotic cell with a pMUT plasmid.

In order to provide a stable expression of exogeneous genes in bacteria, strains are often designed wherein genes are incorporated into the genome of the host cell. This approach provides a stable integration of the gene into the host cell, which simplifies genotyping of the cell and ensures a constant presence of the gene in the cell. Thus, in one aspect of the invention the expression of a sulfotransferase is obtained by genomic integration of the sulfotransferase expressing gene into the probiotic cell.

Genomic integration is to be understood as integration of the gene, nucleic acid sequence construct, promoter, and/or regulatory elements in question into the existing genome of the host cell, wherein the gene is inserted into the chromosome of the host cell. Genomic integration can be done in many ways, such as using endogenous recombinases to insert the gene of interest into a specified homologous plasmid or chromosome of the host cell, such as the pMUT1 or pMUT2 plasmids of E. coli Nissle 1917, wherein the homologous plasmid can be a high copy plasmid or a low copy plasmid.

In embodiments, a sulfotransferase of the present invention may be selected from the sulfotransferases described in table 1.

In one or more embodiments, the engineered probiotic cell of the present invention expresses a sulfotransferase selected from the sulfotransferases of table 1.

The genomic integration of the sulfotransferase gene, sulfatase gene or sulfatase related gene(s) could be obtained using common methods utilized for genomic integration, such as Scarless Cas9 Assisted Recombineering, recombineering, pOSIP one step cloning integration.

In embodiments, the expression of the SULT2A1 is obtained by genomic integration of the human SULT2A1 gene into the probiotic cell. In preferred embodiments, the expression of the codon optimized human SULT2A1 is obtained by genomic integration of the human SULT2A1 gene into the probiotic cell.

In one or more embodiments of the present invention the microbiome-based therapeutic composition comprises an engineered probiotic cell expressing a sulfotransferase, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29.

In an embodiment of the present invention relates to microbiome-based therapeutic composition comprising an engineered probiotic cell expressing a sulfotransferase, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29, wherein the probiotic cell may be a bacterium or yeast, such as but not limited to a bacterium or yeast selected from the group consisting of Roseburia spp., Eubacterium spp., Akkermansia spp., Christensensella spp., Propionibacterium spp., and Faecalibacterium spp, Lactobacillus spp., Bifidobacterium Spp., Streptococcus spp., and Escherichia spp., Saccharomyces spp. In one embodiment the Saccharomyces spp. is Saccharomyces boulardii. In another embodiment the Escherichia spp., is Escherichia coli. In a further embodiment the Escherichia coli is Escherichia coli Nissle 1917.

In an embodiment of the present invention relates to microbiome-based therapeutic composition comprising an engineered probiotic cell expressing a sulfotransferase, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29, wherein the probiotic cell is Escherichia coli Nissle 1917.

In an embodiment of the present invention relates to microbiome-based therapeutic composition comprising an engineered probiotic cell expressing a sulfotransferase, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29, wherein the probiotic cell is Saccharomyces boulardii.

### Regulation of Sulfotransferase and Sulfation Related Genes

Regulatory sequences, herein also referred to as promoters, include those that direct constitutive expression of a nucleotide sequence as well as those that direct inducible expression of the nucleotide sequence only under certain environmental conditions. A bacterial promoter is any DNA sequence capable of binding bacterial RNA polymerase and initiating the downstream (3′) transcription of a coding sequence (e.g., structural gene) into mRNA. A promoter will have a transcription initiation region, which is usually placed proximal to the 5′ end of the coding sequence. This transcription initiation region typically includes an RNA polymerase binding site, and a transcription initiation site. A bacterial promoter may also have a second domain called an operator, which may overlap an adjacent RNA polymerase binding site at which RNA synthesis begins. The operator permits negative regulated (inducible) transcription, as a gene repressor protein may bind to the operator and thereby inhibiting transcription of a specific gene. Constitutive expression may occur in the absence of negative regulatory elements, such as the operator. In addition, positive regulation may be achieved by a gene activator protein binding sequence, which, if present, is usually proximal (5′) to the RNA polymerase binding sequence. The control sequence may be a promoter, a polynucleotide which is recognized by a host cell for expression of the inserted gene or genes. Illustrative, regulator/promoter systems of use for expressing a heterologous polynucleotide in a transformed probiotic cell include without limitation, e.g., Lacl/PT7, Lacl/Ptrc, Pmic7, and/or AraC/PBAD. See, Balzar, et al, Microbial Cell Factories 30 2013, 12:26. Any suitable promoter can be used to carry out the invention including homologous or heterologous promoters. The promoters may be inducible or constitutive such as shown in example 2 and may be operably linked to the gene of interest.

The promoter may be heterologous or homologous with respect to the species of origin relative to the host cell of this invention, or it may be heterologous or homologous with respect to the gene which it promotes (e.g. not the native promoter sequence of the gene to be expressed). Still, with respect to the host cell, the coding DNA may be either heterologous (e.g. derived from another biological species or genus), such as e.g. the DNA sequence encoding human SULT2A1 (SEQ ID NOs: 8-9) or cysP from B. subtilis (SEQ ID NO: 28) expressed in a probiotic cell, such as EcN. In one or more embodiments of the present invention, the engineered probiotic cell expressing a sulfotransferase comprises an inducible or constitutive promoter, that regulates the expression of the sulfotransferase. In other embodiments, the inducible or constitutive promoter regulates the expression of a sulfate permease, or one or more sulfate assimilation pathway relates enzymes. A nucleic acid construct of the invention may be a plasmid DNA carrying the genes to be expressed, or it can in another aspect be an expression cassette/cartridge that is integrated in the genome of a host cell. Accordingly, the term “nucleic acid construct” means an artificially constructed segment of nucleic acid, in particular a DNA segment, which is intended to be genetically engineered into a target cell, e.g. a probiotic cell, in order to modify the expression of a gene or a set of genes of the genome or express one or more genes, which may be included in the construct. In the context of the invention, the nucleic acid construct contains one or more recombinant DNA sequences comprising two or more recombinant DNA sequences: essentially, at least one non-coding DNA sequences comprising one or more promoter DNA sequences and one or more coding DNA sequences encoding one or more genes according to the invention, e.g. T7/lacO promoter sequence (SEQ ID NO: 60), a sulfotransferase, and/or a Pmic promoter (SEQ ID NOs: 61) or a Pmic promoter sequence (SEQ ID NO: 62) and/or a sulfate permease.

In one or more embodiments, the nucleic acid construct comprises one or more recombinant nucleic acid sequences comprising at least one non-coding nuclei acid sequence comprising one or more promoter elements, and further comprises one or more nuclei acid sequences encoding one or more polypeptides according to the present invention. Thus, in an embodiment of the invention the nucleic acid construct comprises a T7/lacO promoter element of SEQ ID NO: 60 placed upstream of a nucleic acid sequence encoding a sulfotransferase, and/or an upstream or downstream Pmic promoter element of SEQ ID NOs: 61 or 62 followed by a downstream or upstream nucleic acid sequence encoding a sulfate permease. Alternatively, the nucleic acid sequences encoding the sulfate permease and the sulfotransferase are placed on two individual constructs, such as but not limited to one or more nucleic acid constructs comprising a T7/lacO promoter element of SEQ ID NO: 60 placed upstream of a nucleic acid sequence encoding a sulfotransferase and a second or further construct comprising a Pmic promoter element of SEQ ID NOs: 61 or 62 followed by nucleic acid sequence encoding a sulfate permease. Methods to combine such nucleic acid sequences into one or more constructs is well known in the art and is considered common general knowledge known to the skilled person.

A construct of the present invention may also comprise additional non-coding DNA sequences that may regulate the expression of the coding nucleic acid sequences.

Preferably, the construct comprises further non-coding DNA sequences that either regulate transcription or translation of the coding DNA of the construct, e.g., a DNA sequence facilitating ribosome binding to the transcript, a leading DNA sequence that stabilize the transcript. Integration of the recombinant gene or genes comprised in the construct into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February;2(2):137-49.); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56 .; Vetcher et al., Appl Environ Microbiol. (2005);71(4): 1829-35); or positive clones, e.g., clones that carry the expression cassette, can be selected e.g. by means of a marker gene, or loss or gain of gene function.

A single copy of the expression cassette comprising a gene of interest may be sufficient to promote the sulfation of secondary bile acids in the GI tract. Accordingly, in some aspects of the current invention, the invention relates to a probiotic cell comprising one, two, three, four, five or six copies of the genes of interest integrated in the genomic DNA of the cell. In some aspects of the invention the single copy of the gene is preferred, while in some aspect more copies of the gene are preferred.

Thus, the microbiome-based therapeutic composition comprising a probiotic cell expressing the sulfotransferase can—within the encoding plasmid or integrated gene—comprise an inducible promoter, as shown in SEQ ID NO: 60. Furthermore, the microbiome-based therapeutic composition comprising a probiotic cell expressing the sulfotransferase, can—within the encoding plasmid or integrated gene—also comprise a constitutive promoter, as shown in SEQ ID NO: 61 and/or SEQ ID NO: 62.

Thus, in one or more embodiments, the probiotic cell expressing the sulfotransferase comprises one or more nucleic acid sequences comprising a non-coding promoter sequences of any one of SEQ ID NOs: 60-62.

The genetic material, plasmid or otherwise encoding a sulfotransferase can contain a single gene to be transcribed or it can contain multiple genes to be transcribed within the probiotic cell. Therefore, the probiotic cell can contain regulation of several of the genes involved with sulfation of secondary bile acids and xenobiotics. Examples are sulfate transport genes responsible for the transport of sulfate into the bacterial lumen, such as the sulfate permeases. The sulfate permeases can be endogenous to the probiotic cell, or an exogeneous sulfate permease can be integrated in the plasmid or genome. Thus, in relation to the invention, the plasmid encoding the sulfotransferase, or the genome of the probiotic cell expressing a sulfotransferase can further comprise an exogeneous sulfate permease gene. In embodiments of the invention, the cell further expresses one or more genes resulting in an increased sulfate uptake. Thus, in embodiments of the invention, the plasmid encoding the sulfotransferase, or the genome of the probiotic cell further comprises a Bacillus subtilis cysP gene, as shown in SEQ ID NO: 28.

Since the degree of sulfation of secondary bile acids and xenobiotics relates not only to the sulfotransferases and the sulfate permeases, other parts of the sulfation machinery (related to sulfate maturation) of the probiotic cell could also be optimized towards enhancing sulfation of secondary bile acids and xenobiotics. Examples include sulfate permease related genes or sulfate maturation related genes. Thus, in embodiments of the invention the probiotic cell could be further genetically engineered, so that the expression of one or more genes, selected from the group consisting of cysZ, sbp, cysP, cysU, cysW, cysA, cysD, cysN, cysC and cysQ, (SEQ ID NOs: 20-27) encoding one or more sulfate permeases, sulfate permease related genes or sulfate recycling related genes, is/are upregulated. The cysZ, sbp, cysP, cysU, cysW, and cysA, genes are the sulfate uptake genes of E. coli and cysD, cysN, cysC and cysQ relates to sulfate recycling in E. coli.

Example 5 shows that inactivation of cysQ have a significant effect on the level of sulfation obtained for both LCA and DCA sulfation. In that regard, cysQ may be inactivated in the probiotic cell of the present invention.

The inventors demonstrated in example 3 that upregulation of these genes modulate the sulfation of the secondary bile acids, especially in combination with expression of the cysP gene from B. subtilis (SEQ ID NO: 28).

Thus, the probiotic cell can be further genetically engineered, so that the expression of one or more genes, selected from the group consisting of cysP, cysU, cysW, cysA, cysD, cysN, cysC and cysQ, encoding one or more sulfate permeases, sulfate permease related polypeptides or sulfate recycling related polypeptides of SEQ ID NOs: 44-51, is/are regulated by a promoter, wherein the protein of any one of SEQ ID NOs: 44-51, or a functional homologue thereof, has at least 80%, such as at least 85%, at least 90%, at least 95%, at least 96%, at least 97%, at least 98% or at least 99% sequence similarity to any one of SEQ ID NOs: 44-51 and wherein a functional homologue of any one of SEQ ID NOs: 44-51, has at least 50%, such as at least 75%, such as at least 85%, at least 90%, at least 95%, at least 96%, at least 97%, at least 98%, at least 99%, 100% or such as above 100% functionality of said protein. Thus, in particular, the probiotic cell can be further genetically engineered, so that the expression of one or more genes, selected from the group consisting of cysP, cysU, cysW and cysA, encoding one or more sulfate permeases, sulfate permease related proteins or sulfate recycling related proteins of SEQ ID NOs: 44-47, is/are upregulated, wherein the protein of any one of SEQ ID NOs: 44-47, or a functional homologue thereof, has at least 80%, such as at least 85%, at least 90%, at least 95%, at least 96%, at least 97%, at least 98%, such as at least 99% sequence similarity to any one of SEQ ID NOs: 44-47 and wherein a functional homologue of any one of SEQ ID NOs: 44-47, has at least 50%, such as at least 75%, at least 80%, at least 85%, at least 90%, at least 95%, at least 96%, at least 97%, at least 98%, at least 99% such as 100% or such as above 100% functionality of said protein. In particular, the probiotic cell can be further genetically engineered, so that the expression of one or more genes, selected from the group consisting of cysD, cysN, cysC and cysQ, encoding one or more sulfate recycling related genes, is/are upregulated encoding one or more sulfate permeases, sulfate permease related proteins or sulfate recycling related proteins of SEQ ID NOs: 48-51, is/are upregulated, wherein the protein of any one of SEQ ID NOs: 48-51, or a functional homologue thereof, has at least 80%, such as at least 90%, such as 95% such as 99% sequence similarity to any one of SEQ ID NOs: 48-51 and wherein a functional homologue of any one of SEQ ID NOs: 48-51, has at least 50%, such as at least 75%, at least 85%, at least 90%, at least 95%, at least 96%, at least 97%, at least 98%, at least 99% such as 100% or such as above 100% functionality of said protein.

Thus, the probiotic cell can be further genetically engineered, so that the expression of one or more genes, selected from the group consisting of cysP, cysU, cysW, cysA, cysD, cysN, cysC, encoding one or more sulfate permeases, sulfate permease related polypeptides or sulfate recycling related polypeptides of SEQ ID NOs: 44-51, is/are regulated by a promoter, wherein the protein of any one of SEQ ID NOs: 44-51, or a functional homologue thereof, has at least 80%, such as at least 85%, at least 90%, at least 95%, at least 96%, at least 97%, at least 98% or at least 99% sequence similarity to any one of SEQ ID NOs: 44-51 and wherein a functional homologue of any one of SEQ ID NOs: 44-51, has at least 50%, such as at least 75%, such as at least 85%, at least 90%, at least 95%, at least 96%, at least 97%, at least 98%, at least 99%, 100% or such as above 100% functionality of said protein. Thus, in particular, the probiotic cell can be further genetically engineered, so that the expression of one or more genes, selected from the group consisting of cysP, cysU, cysW and cysA, encoding one or more sulfate permeases, sulfate permease related proteins or sulfate recycling related proteins of SEQ ID NOs: 44-47, is/are upregulated, wherein the protein of any one of SEQ ID NOs: 44-47, or a functional homologue thereof, has at least 80%, such as at least 85%, at least 90%, at least 95%, at least 96%, at least 97%, at least 98%, such as at least 99% sequence similarity to any one of SEQ ID NOs: 44-47 and wherein a functional homologue of any one of SEQ ID NOs: 44-47, has at least 50%, such as at least 75%, at least 80%, at least 85%, at least 90%, at least 95%, at least 96%, at least 97%, at least 98%, at least 99% such as 100% or such as above 100% functionality of said protein. In particular, the probiotic cell can be further genetically engineered, so that the expression of one or more genes, selected from the group consisting of cysD, cysN and cysC, encoding one or more sulfate recycling related genes, is/are upregulated encoding one or more sulfate permeases, sulfate permease related proteins or sulfate recycling related proteins of SEQ ID NOs: 48-51, is/are upregulated, wherein the protein of any one of SEQ ID NOs: 48-51, or a functional homologue thereof, has at least 80%, such as at least 90%, such as 95% such as 99% sequence similarity to any one of SEQ ID NOs: 48-51 and wherein a functional homologue of any one of SEQ ID NOs: 48-51, has at least 50%, such as at least 75%, at least 85%, at least 90%, at least 95%, at least 96%, at least 97%, at least 98%, at least 99% such as 100% or such as above 100% functionality of said protein.

Sulfation of secondary bile acids and xenobiotics is dependent on the activity of the endogenous sulfatases, a class of enzymes that catalyse the hydrolysis of sulfate esters. In the case of sulfated secondary bile acids and the xenobiotics, the role of the sulfatases is to hydrolyse the sulfate esters, resulting in desulfation, thereby reducing the production of sulfated secondary bile acids and xenobiotics. Thus, the probiotic cell can be further genetically engineered so that the sulfatases involved in desulfation of the secondary bile acids and the xenobiotics is inhibited. In one aspect of the invention, the probiotic cell is further genetically engineered so that one or more of the sulfatase and/or sulfatase related gene(s) is/are at least partially inactivated. As shown in example 4, the inventors have found that the sulfatase related genes yjcS, aslA, ydeN, yidJ, ydeM, aslB, and hdhA (SEQ ID Nos: 53-59) was interesting targets to enhance sulfation due to a lowered sulfate metabolism. Thus, the probiotic cell may be further genetically engineered so that one or more of the sulfatases and/or sulfatase related gene(s), selected from the group consisting of yjcS, aslA, ydeN, yidJ, ydeM asIB, and hdhA is/are at least partially inactivated.

Specifically, the inventors found (example 4) that a subset of the sulfate related genes was especially efficient in enhancing the level of sulfation of the secondary bile acids. Thus, the probiotic cell may be further genetically engineered so that one or more of the sulfatase and/or sulfatase related gene(s), selected from the group consisting of ydeN, ydeM asIB, and hdhA is/are at least partially inactivated.

In an embodiment the present invention relates to microbiome-based therapeutic composition comprising an engineered probiotic cell expressing a sulfotransferase, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29, and wherein one or more endogenous genes selected from the group consisting of yjcS, aslA, ydeN, yidJ, ydeM aslB, hdhA, cysH, cysQ and acrB is/are at least partially inactivated.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease selected from the group consisting of cysP from E. coli and cysP from Bacillus subtilis, with an amino acid sequence according to SEQ ID NOs: 44 and 52, and
   wherein one or more endogenous genes selected from the group consisting of yjcS, asA, ydeN, yidJ, ydel astB, hdhA, cyst, cysQ and acrB is/are at least partially inactivated, and said probiotic coll is Escherichia Coli Nisale 1917.

References describing the nucleic acid sequences encoding each of yjcS, astA, ydeN, yidJ, ydoM aslB, hdhA, cyaH, cysQ and acrB are provided in table 2.

TABLE 2
Nucleic acid sequences encoding the targeted knockout genes

| | | NCBI identifier/ |
|---|---|---|
| Gene | Function | locus ID |
| cysH | phosphoadenosine | ECK2757/b2762 |
| | phosphosulfate reductase | |
| cysD | sulfate adenylyltransferase | ECK2747/b2752 |
| | subunit 2 | |
| cysN | sulfate adenylyltransferase | ECK2746/b2751 |
| | subunit 1 | |
| cysC | adenylyl-sulfate kinase | ECK2745/b2750 |
| cysP | thiosulfate/sulfate | ECK2420/b2425 |
| | ABC transporter | |
| | periplasmic binding protein | |
| cysU | sulfate/thiosulfate ABC transporter | ECK2419/b2424 |
| | inner membrane subunit | |
| cysW | sulfate/thiosulfate ABC transporter | ECK2418/b2423 |
| | inner membrane subunit | |
| cysA | sulfate/thiosulfate ABC transporter | ECK2417/b2422 |
| | ATP binding subunit | |
| cysZ | sulfate:H+ symporter | ECK2408/b2413 |
| cysQ | 3′(2′),5′-bisphosphate | ECK4210/b4214 |
| | nucleotidase | |
| ssuA | aliphatic sulfonate ABC transporter | ECK0927/b0936 |
| | periplasmic binding protein | |
| ssuB | aliphatic sulfonate ABC transporter | ECK0924/b0933 |
| | ATP binding subunit | |
| ssuC | aliphatic sulfonate ABC transporter | ECK0925/b0934 |
| | membrane subunit | |
| ssuD | FMNH2-dependent alkanesulfonate | ECK0926/b0935 |
| | monooxygenase | |
| yidF | uncharacterized protein YidF | ECK3665/b3674 |
| | (putative transcriptional regulator) | |
| yidG | inner membrane protein | ECK3666/b3675 |
| yidH | DUF202 domain-containing | ECK3667/b3676 |
| | inner membrane protein | |
| acrB | multidrug efflux pump | ECK0456/b0462 |
| | RND permease | |
| emrA | multidrug efflux pump | ECK2679/b2685 |
| | membrane fusion protein | |
| emrB | multidrug efflux pump | ECK2680/b2686 |
| | membrane subunit | |
| ptsl | phosphoenolpyruvate-protein | ECK2411/b2416 |
| | phosphotransferase | |
| sbp | sulfate/thiosulfate | ECK3909/b3917 |
| | ABC transporter | |
| | periplasmic binding | |
| | protein | |

*Nucleic acid sequences are available at https://www.ncbi.nlm.nih.gov/gene using the above NCBI identifier or locus tag.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease selected from the group consisting of cysP from E. coli and cysP from Bacillus subtilis, with an amino acid sequence according to SEQ ID NOs: 44 and 52, and wherein one or more endogenous genes ydeN, cysH, cysQ and/or acrB is/are at least partially inactivated, and said probiotic cell is Escherichia Coli Nissle 1917.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95%, such as 99% sequence identity to SEQ ID NO: 29 and
b) a sulfate permease, cysP from E. coli, with an amino acid sequence according to SEQ ID NO: 44 or a functional homologue thereof, having at least 80% sequence identity, such as 90%, such as 95% such as 99% sequence identity to SEQ ID NO: 44, and wherein one or more endogenous genes ydeN, cysH, cysQ and/or acrB is/are at least partially inactivated, and said probiotic cell is Escherichia coli Nissle 1917.

Accordingly, in embodiments one or more endogenous genes ydeN, cysH, cysQ and/or acrB, with a nucleic acid sequence according to SEQ ID NOs: 55, 69, 70 and 71 respectively, is/are at least partially inactivated, in said engineered probiotic cell.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease, cysP from Bacillus subtilis, with an amino acid sequence according to SEQ ID NO: 52 or a functional homologue thereof, having at least 80% sequence identity, such as 90%, such as 95% such as 99% sequence identity to SEQ ID NO: 52, and
   wherein one or more endogenous genes ydeN, cysH, cysQ and/or acrB is/are at least partially inactivated, and said probiotic cell is Escherichia coli Nissle 1917.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease, cysP from Bacillus subtilis, with an amino acid sequence according to SEQ ID NO: 52 or a functional homologue thereof, having at least 80% sequence identity, such as 90%, such as 95% such as 99% sequence identity to SEQ ID NO: 52, and
   wherein the endogenous gene ydeN is at least partially inactivated, and said probiotic cell is Escherichia coli Nissle 1917.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease, cysP from Bacillus subtilis, with an amino acid sequence according to SEQ ID NO: 52 or a functional homologue thereof, having at least 80% sequence identity, such as 90%, such as 95% such as 99% sequence identity to SEQ ID NO: 52, and wherein the endogenous gene cysH is at least partially inactivated, and said probiotic cell is Escherichia coli Nissle 1917.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease, cysP from Bacillus subtilis, with an amino acid sequence according to SEQ ID NO: 52 or a functional homologue thereof, having at least 80% sequence identity, such as 90%, such as 95% such as 99% sequence identity to SEQ ID NO: 52, and wherein the endogenous genes cysQ is at least partially inactivated, and said probiotic cell is Escherichia coli Nissle 1917.

In an embodiment the present invention relates to microbiome-based therapeutic composition comprising an engineered probiotic cell expressing;
a) a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and
b) a sulfate permease, cysP from Bacillus subtilis, with an amino acid sequence according to SEQ ID NO: 52 or a functional homologue thereof, having at least 80% sequence identity, such as 90%, such as 95% such as 99% sequence identity to SEQ ID NO: 52, and wherein the endogenous gene acrB is at least partially inactivated, and said probiotic cell is Escherichia coli Nissle 1917.

In an embodiment the present invention relates to microbiome-based therapeutic composition comprising an engineered probiotic cell expressing a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and wherein the endogenous genes yjcS, ydeN, ydeM aslB, hdhA, cysH, cysQ, acrB, yidF, yidG, yidH and/or emrA is at least partially inactivated, and said probiotic cell is Escherichia coli Nissle 1917.

In an embodiment the present invention relates to microbiome-based therapeutic composition comprising an engineered probiotic cell expressing a sulfotransferase, wherein the sulfotransferase is human SULT2A1, with an amino acid sequence according to SEQ ID NO: 29, or a functional homologue thereof, having at least 80% sequence identity, such as 90%, such as 95% such as 99% sequence identity to SEQ ID NO: 29 and wherein the endogenous genes ydeN, cysH and/or cysQ is at least partially inactivated, and said probiotic cell is Escherichia coli Nissle 1917.

In an or more exemplified embodiments, the yjcS gene of the engineered probiotic cell of the present invention is at least partially inactivated. In one or more exemplified embodiments, the ydeN gene of the engineered probiotic cell of the present invention is at least partially inactivated. In one or more exemplified embodiments, the ydeM gene of the engineered probiotic cell of the present invention is at least partially inactivated. In one or more exemplified embodiments, the as/B gene of the engineered probiotic cell of the present invention is at least partially inactivated. In one or more exemplified embodiments, the hdhA gene of the engineered probiotic cell of the present invention is at least partially inactivated. In one or more exemplified embodiments, the cysH of the engineered probiotic cell of the present invention is at least partially inactivated. In one or more exemplified embodiments, the cysQ gene of the engineered probiotic cell of the present invention is at least partially inactivated. In one or more exemplified embodiments, the acrB gene of the engineered probiotic cell of the present invention is at least partially inactivated.

In one or more embodiments at least one sulfate assimilation pathway related gene is knocked out in the engineered probiotic cell expressing a sulfotransferase, such as at least two genes, such as at least three genes, such as at least four genes or such as at least five genes.

As mentioned above, example 4 shows examples on genes that enhance the level of sulfated secondary bile acids when knocked out (FIG. 11). However, as shown in FIG. 11, some genes, such as ssuB, emrB and pstl abolish the sulfation of the secondary bile acids when knocked out. This could to some extend indicate that overexpression of the said genes could be used to enhance sulfation of the secondary bile acids. Thus, in one or more embodiments of the present invention, one or more of the genes ssubB, emrB and pstl, are overexpressed in the engineered probiotic cell. Overexpression of said genes may be obtained by any method known to the skilled person e.g., using promoter swapping or inclusion of additional genetic copies of said gene(s). Non-limiting examples of suitable promoters are provided herein.

Accordingly, in embodiments one or more endogenous genes ssuB, emrB, and/or pstl, with a nucleic acid sequence according to SEQ ID NOs: 72, 73 and 74 respectively, are overexpressed in the engineered probiotic cell. In embodiments the endogenous gene ssuB with a nucleic acid sequence according to SEQ ID NOs: 72 is overexpressed in the engineered probiotic cell. In embodiments the endogenous gene emrB with a nucleic acid sequence according to SEQ ID NOs: 73 is overexpressed in the engineered probiotic cell. In embodiments the endogenous gene pst/with a nucleic acid sequence according to SEQ ID NOs: 74 is overexpressed in the engineered probiotic cell.

Partially inactivated is to be understood as a genetic modification reducing the functionality and/or expression of the indicated gene or protein, thus a partial inactivation can be a functionality of less than 100% compared to the normal functionality of said gene/protein, such as less than 90%, 80%, 70%, 50%, 40%, 20% or less than 10% such as less than 1% functionality. Inactivated, in relation to a gene may refer to inclusion of a stop codon or frame shift into the gene or deletion of the gene or otherwise genetically modified, which reduces or abolishes the functionality of said gene or genes. DNA techniques for full or partial inactivation of genes using genetic modification is well known in the art and are all parts of the knowledge of the skilled person.

The genetic modifications can for example be selected from inclusion of the human SULT2A1, and/or sulfate pathway engineering, and inclusion of sulfate transporters as described in the above sections, which the skilled person will know how to combine into a genetically engineered probiotic cell of the present invention.

### A Process for Preparing/Producing a Microbiome-based Therapeutic Composition Comprising an Engineered Probiotic Cell

The invention also relates to the process for preparing an engineered probiotic cell for use in a microbiome-based therapeutic composition.

The process for preparing an engineered probiotic can comprise a step for preparing the host cell such as EcN for transformation with a plasmid or for genetic integration. The process furthermore contains a step for preparing the plasmid, construct or gene for transformation or genomic integration. The process could include a step for transforming the probiotic cell with a plasmid or construct, encoding the sulfotransferase and/or additional genes, such as, but not limited to a sulfate permease, cofactor recycling genes and/or sulfate maturation genes, as well as one or more promoter sequences. Alternatively, the process could contain a step for integrating the gene encoding the sulfotransferase and/or additional genes, such as, but not limited to a sulfate permease, cofactor recycling genes and/or sulfate maturation genes, as well as one or more promoter sequences, into the genome of the probiotic cell. The process also contains a step for selecting the genetically engineered probiotic cell over a non-genetically engineered probiotic cell. Said selection step could comprise, antibiotic selection or nutritional selection. The genetically engineered probiotic cell can be subjected to validation in order to confirm their genus/species identity, the absence of pathogenic toxins and susceptibility to clinically used antibiotics. For example, using PCR, qPCR, next-generation sequencing and/or Sanger sequencing, e.g., of all or part of the genome sequence, can be performed to confirm genus/species identity.

To ensure that the genetically engineered probiotic cell is excreted from the subject, or in the case of side effects, the probiotic cell may be tested for antibiotic resistance prior to administration to a subject.

Thus, in embodiments of the invention the genetically engineered probiotic cell is confirmed to be sensitive or susceptible (e.g., lack resistance) to one or more antibiotic agents selected from the group consisting of antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, or gemifloxacin), β-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), and/or tetracyclines (e.g., tetracycline, chlortetracycline, oxytetracycline, demeclocycline, lymecycline, meclocycline, methacycline, minocycline, rolitetracycline, or tigecycline). Generally, transformation of the purified native bacterial colony with a heterologous polynucleotide confers antibiotic resistance to one or more antibiotic agents used for selection of transformed probiotic cells, e.g,., resistance to kanamycin, chloramphenicol, carbenicillin, hygromycin and/or trimethoprim. Genetic integration of the heterologous polynucleotide into the genome of the probiotic cell, can in one aspect of the invention confer antibiotic resistance to one or more antibiotic agents used for selection of the probiotic cell. In another aspect the invention the genetic integration, of the heterologous polynucleotide into the genome of the probiotic cell, does not confer antibiotic resistance to one or more antibiotic agents used for selection of the probiotic cell.

The process also includes a step for culturing the genetically engineered probiotic cell, such as in a shaker culture or in a bioreactor, such as a fermenter or bacterial culture tank, or in a sponge culture. Following cultivation, the process contains a step for harvesting the genetically engineered probiotic cell, the harvesting method could be any common method for harvesting bacterial cultures, such as centrifugation, microfiltration, membrane cross flow microfiltration, ultrafiltration, harvest by viafuge, sedimentation or flocculation, freeze drying and/or direct spray drying. The process might also contain a combination of the above methods for harvesting. The harvested genetically engineered probiotic cell culture may be stored, in any common manner known to the skilled person, and/or further processed in order to prepare the cell for administration. Further processing could contain drying, granulation, powdering, micronization, resuspension or other methods known to the skilled person.

The microbiome-based therapeutic may be delivered as a lyophilized (freeze-dried) powder packaged in a consumable capsule. One process for preparing a lyophilized powder of the microbiome-based therapeutic of the invention may be prepared as described in brief; the liquid culture can be: centrifuged, resuspended in a lyophilization medium which optionally can include cryoprotectants and biological- and/or chemical-oxygen scavengers transferred under anaerobic conditions to a lyophilizer, lyophilized, encapsulated in a capsule under anaerobic conditions, and packaged in a glass ampoule to maintain oxygen free conditions during transport and storage. The robustness of the microbiome-based therapeutic over time can be assessed using different configurations containing single and various factorial mixtures of excipients prepared via the same lyophilization, encapsulation, and packaging procedures. Products can then be stored in a laboratory setting on a shelf at room temperature, in a refrigerator or in a freezer and tested for viability at 0, 30, 60, 180, and 360 days from the date of production. Validation can be performed by breaking an ampoule under aerobic conditions (as would be encountered when delivering the capsule to a subject in a medical setting) and then placing the capsule in a suitable media, such as M9 medium (table 3 and table 4, example 1).

### Use of the Microbiome-based Therapeutic in Medicine and a Dosage Regimen for the Microbiome-based Therapeutic

In one aspect of the invention the expression of a sulfotransferase results in an enhanced sulfation of secondary bile acids. In a preferred aspect of the invention, the microbiome-based therapeutic composition is for use in medicine. The therapeutic composition comprises an engineered probiotic cell expressing under promoter regulation, a sulfotransferase and/or a sulfate permease and/or sulfate recycling or maturation genes. Especially, the microbiome-based therapeutics can be given as prophylactic treatment and for treatment of diseases.

In a further aspect of the invention, the microbiome-based therapeutic composition according to the invention is given as a treatment of diseases. The secondary bile acids, as disclosed herein, play a role in intestinal cancer, colorectal cancer and hepatic cancer, as well as intestinal inflammation, hepatic inflammation, cirrhosis, hepatic diseases, gall stones, non-alcoholic fatty liver disease (NAFLD), non-alcoholic steatohepatitis (NASH), fatty liver, cholestasis, hepatic fibrosis, hepatitis, inflammatory bowel disease (IBD), Crohn's disease, ulcerative colitis, wherein the amount of bile acids is maladapted in a way that promotes the disease state. Some bile acids are also known to cross the blood brain barrier (BBB), and act on receptors in the central nervous system, which has been implicated in the development of Parkinson's disease, wherein bile acid levels in patients in a pre-onset Parkinson stage is in some cases elevated. Therefore, a reduction in the amount of bile acids and secondary bile acids through sulfation, can be beneficial in the treatment of the disease, since the sulfation of the bile acids promotes excretion of the secondary bile acid. Accordingly, in one or more embodiments, the microbiome-based therapeutic composition of the present invention is for use as a medicament. Furthermore, in one or more additional embodiments, the microbiome-based therapeutic composition of the present invention is for use in the treatment of cancer and/or inflammatory diseases. Thus, the microbiome-based therapeutic composition according to the invention may be used in the treatment of one or more of diseases, such as intestinal cancer, colorectal cancer and hepatic cancer, as well as intestinal inflammation, hepatic inflammation, cirrhosis, hepatic diseases, gall stones, NAFLD, NASH, fatty liver, cholestasis, hepatic fibrosis, hepatitis, IBD, Crohn's disease, ulcerative colitis. In preferred embodiments, the microbiome-based therapeutic composition according to the invention is used in the treatment of intestinal inflammation, intestinal cancer, colorectal cancer and/or hepatic cancer.

Prophylactic treatment can be given to prevent spreading or occurrence of a disease or infection. In the present context, a prophylactic treatment is a treatment that is given to prevent diseases originating from maladapted levels of secondary bile acids, wherein the prophylactic treatment comprises administering to a subject in need thereof a microbiome-based therapeutic composition comprising an engineered probiotic cell expressing at least a sulfotransferase. Secondary bile acids are also seen as tumor promoters in intestinal cancers, where especially DCA and LCA induce oxidative stress and DNA damage, which may result in tumor progression. Thus, the microbiome-based therapeutic composition according to the invention can be provided as prophylactic treatment. Accordingly, the microbiome-based therapeutic composition for use according to the present invention is for use in the treatment of colon cancer. In another embodiment, the microbiome-based therapeutic composition for use according to the present invention is for use in ameliorating cancer and/or inflammatory disease(s). In a further embodiment, the microbiome-based therapeutic composition for use according to the present invention is for use in ameliorating colon cancer. In another embodiment, the microbiome-based therapeutic composition for use according to the present invention is for use in inhibiting cancer and/or inflammatory disease(s). In a further embodiment, the microbiome-based therapeutic composition for use according to the present invention, is for use in inhibiting colon cancer.

In a further embodiment, the microbiome-based therapeutic composition for use according to the present invention is for use in the treatment of a metabolic disorder.

The level of secondary bile acids can be maladapted for several reasons, such as a high fat diet, that promotes synthesis of secondary bile acids, and the maladaptation might be reversed over a period of time or in a single treatment. Therefore, the microbiome-based therapeutic composition of the invention, is administered once or repeatedly. Also, as a consequence of the excretion of the microbiome-based therapeutic composition of the present invention, the treatment is likely to require multiple dosing. As is described in FIGS. 16-18, the microbiome-based therapeutic composition may be administered to the host by repeated dosing, in the figure, once every second day. Accordingly, in one or more embodiment, the microbiome-based therapeutic composition of the present invention is administered repeatedly to the host, such as daily, weekly, or monthly, depending on the host and the specific transit/retention times and/or excretion profile.

The microbiome-based therapeutic composition of the invention may be used in bacterial sulfation of secondary bile acids for treating and/or preventing bile acid disorders and/or complications resulting from and/or leading to unbalanced bile acid pools.

Moreover, the microbiome-based therapeutic composition according to the invention may be used in bacterial sulfation of secondary bile acids for treating and/or preventing bile acid disorders and/or complications resulting from and/or leading to unbalanced bile acid pools.

Secondary bile acids are the result of partial dehydroxylation, and in some cases also oxidation, of one or more of the secondary hydroxyl groups in the primary bile acids such as the bile acids derived from cholic acid (CA) and chenodeoxycholic acid (CDCA). The dehydroxylation, and potential oxidation of CA and CDCA, potentially produces a number of derivates of the primary bile acids. Additionally, the secondary bile acids may also be conjugated with, in example, an amino acid, such as but not limited to glycine, taurine, phenylalanine, tyrosine or leucine at the carboxylic acid functional group in a condensation reaction between the carboxylic acid of the bile acid and the amine of the amino acid to form a conjugated primary or secondary bile acid, such as but not limited to taurolithocholic acid (TLCA), glycolithocholic acid (GLCA) and glycodeoxycholic acid (GDCA). Conjugates of the secondary bile acids are also referred to as derivates of secondary bile acids.

Accordingly in an embodiment, the secondary bile acids of the present invention may also comprise conjugates of the secondary bile acid, such as but not limited to taurolithocholic acid (TLCA), glycolithocholic acid (GLCA) and glycodeoxycholic acid (GDCA).

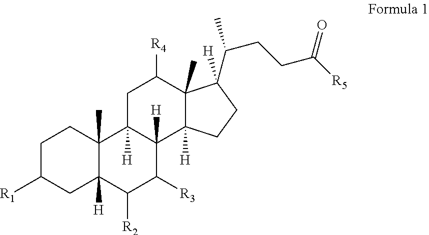

Thus in an embodiment, the secondary bile acids of the present invention are derived from primary bile acids, wherein said secondary bile acids are of the general formula 1, wherein, R1, R2, R3, and R4 may be OH, O or H and R5 is OH, O or NH conjugated with for instance, an amino acid, such as but not limited to glycine, taurine, phenylalanine, tyrosine or leucine and wherein at least one of R₁, R₂, R₃, or R₄ is H or O while one or two of R₁, R₂, R₃ or R₄ is OH.

In that regard, in one or more embodiments, the secondary bile acid(s) sulfated by the engineered probiotic cell of the present invention is/are selected from the group consisting of LCA, DCA, UDCA, HDCA, GLCA, GDCA, GUDCA, TLCA, TDCA, TUDCA, THDCA and conjugates thereof.

Due to the stereochemical variants of the bile acid sterol backbone presented in formula 1 the R₁, R₂, R₃, and R₄, along with H and CH₃ groups exist in different isomeric variants, accordingly the above-mentioned secondary bile acids may also be found as isomeric variants of the secondary bile acids, such as but not limited to isoLCA, isoDCA, alloLCA, alloDCA and derivates thereof.

Additionally, the microbiome-based therapeutic composition according to the invention may be used in bacterial sulfation of secondary bile acids for treating and/or preventing bile acid disorders and/or complications resulting from and/or leading to unbalanced bile acid pools, wherein said secondary bile acids are selected from the group consisting of LCA, DCA, TDCA, GDCA, GLCA, UDCA, TLCA, TUDCA and GUDCA.

The microbiome-based therapeutic composition according to the invention can also be used in bacterial sulfation of secondary bile acids for treating and/or preventing bile acid disorders and/or complications resulting from and/or leading to unbalanced bile acid pools, the secondary bile acids are LCA and DCA.

In one or more exemplified embodiments, the ydeN gene of the engineered probiotic cell of the present invention is at least partially inactivated wherein the inactivation of ydeN enhances sulfation of DCA at least 1.1-fold, such as but not limited to at least 2-fold, such as but not limited to at least 3-fold compared to an engineered probiotic cell expressing ydeN.

In one or more exemplified embodiments, the ydeM gene of the engineered probiotic cell of the present invention is at least partially inactivated, wherein the inactivation of ydeM enhances sulfation of DCA at least 1.1-fold, such as but not limited to at least 1.5-fold, such as but not limited to at least 2-fold compared to an engineered probiotic cell expressing ydeM.

In one or more exemplified embodiments, the asIB gene of the engineered probiotic cell of the present invention is at least partially inactivated wherein the inactivation of asIB enhances sulfation of DCA at least 1.1-fold, such as but not limited to at least 1.5-fold, such as but not limited to at least 1.75-fold, such as but not limited to at least 2-fold compared to an engineered probiotic cell expressing aslB.

In one or more exemplified embodiments, the hdhA gene of the engineered probiotic cell of the present invention is at least partially inactivated wherein the inactivation of hdhA enhances sulfation of DCA at least 1.1-fold, such as but not limited to at least 1.5-fold, such as but not limited to at least 1.75-fold, such as but not limited to at least 2.2-fold compared to an engineered probiotic cell expressing hdhA.

In one or more exemplified embodiments, the cysH gene of the engineered probiotic cell of the present invention is at least partially inactivated wherein the inactivation of cysH enhances sulfation of DCA at least 2-fold, such as but not limited to at least 5-fold, such as but not limited to at least 10-fold, such as but not limited to at least 14-fold compared to an engineered probiotic cell expressing cysH.

In one or more exemplified embodiments, the cysQ gene of the engineered probiotic cell of the present invention is at least partially inactivated wherein the inactivation of cysQ enhances sulfation of DCA at least 5-fold, such as but not limited to at least 10-fold, such as but not limited to at least 15-fold, such as but not limited to at least 20-fold compared to an engineered probiotic cell expressing cysQ.

In one or more exemplified embodiments, the acrB gene of the engineered probiotic cell of the present invention is at least partially inactivated wherein the inactivation of acrB enhances sulfation of DCA at least 2-fold, such as but not limited to at least 3-fold, such as but not limited to at least 4-fold compared to an engineered probiotic cell expressing acrB.

In one or more exemplified embodiments, the cysH gene of the engineered probiotic cell of the present invention is at least partially inactivated wherein the inactivation of cysH enhances sulfation of LCA at least 2-fold, such as but not limited to at least 3-fold, such as but not limited to at least 4-fold compared to an engineered probiotic cell expressing cysH.

In one or more exemplified embodiments, the cysQ gene of the engineered probiotic cell of the present invention is at least partially inactivated wherein the inactivation of cysQ enhances sulfation of LCA at least 2-fold, such as but not limited to at least 3-fold, such as but not limited to at least 4-fold compared to an engineered probiotic cell expressing cysQ.

Another aspect of the microbiome-based therapeutic composition according to the invention relates to the sulfation of xenobiotics, such as the catecholamines and serotonin, wherein these are susceptible to sulfation of the hydroxyl groups, e.g. both the hydroxy groups of dopamine are known to undergo sulfation. Sulfation of the catecholamines alters the chemical properties of the molecules and can affect the solubility, permeability and stability of the catecholamines. Sulfation of dopamine is known to enhance the blood brain barrier permeability of dopamine, which is otherwise blood brain barrier impermeable. Thus, the microbiome-based therapeutic composition according to the invention can also be used for enhancing the bioavailability of xenobiotic and native catecholamines and serotonin. In one aspect of the invention the microbiome-based therapeutic composition is for use in the treatment of neurological disorders and/or metabolic disorders. In a further aspect of the invention the microbiome-based therapeutic composition is used in the treatment of disorders related to maladapted serotonin levels, such as but not limited to metabolic disorders, such as obesity, diabetes, arteriosclerosis, hypertension, cardiovascular disease, impaired glucose homeostasis and/or insulin resistance, such as diabetes, sleep disorders, psychiatric disorders such as depression, anxiety, psychosis and/or schizophrenia. Another aspect of the invention relates to the use of the use of the microbiome-based therapeutic composition in the treatment of diseases related to maladapted dopamine or serotonin homeostasis and/or dopamine or serotonin levels, such as Parkinson disease, depression, attention-deficit hyper-activity disorder, schizophrenia and dementia.

A special feature of microbiome-base therapeutic, wherein the probiotic strain is EcN, is that upon cessation of the treatment, the microbiome-base therapeutic will be cleared from the subject with a timeframe of from 1 day to 35 days, such as 2 days to 28 days, such as 3 days to 21 days or such as 4 days to 14 days. Furthermore, the microbiome-based therapeutic composition according to the invention can be administered in a dose of 10⁸ to 10¹¹ CFU pr. day, such as 10⁹ to 9×10¹⁰, or more preferably such as 5×10⁹ to 8×10¹⁰, even more preferably 1×10¹⁰ to 7.5×10¹⁰. In one aspect of the invention the microbiome-based therapeutic composition according to the invention is administered in a dose of 1.0×10¹⁰ to 7.5×10¹⁰ CFU pr. Day. In one aspect of the invention, the amount and effect of the microbiome-based therapeutic in a human is monitored, by specific detection of gut microbes (e.g., by 16S rRNA gene sequencing or 16S rRNA copy number). The microbiome-based therapeutic composition is intended for use in the gastrointestinal tract. Thus, the composition could be designed for administration by oral or rectal administration. Alternatively the composition could be designed for administration by faecal microbiota transplantation for direct dosing in the intestine. The composition can be provided as a tablet, capsule or suppository or other dosage forms relevant for oral or rectal administration.

The invention is further illustrated in the following non-limiting figures and examples.

### BRIEF DESCRIPTION OF THE FIGURES

FIG. 1 shows the sulfation of secondary bile acids in E. coli. FIG. 1A, shows sulfation of secondary bile acids, DCA and LCA, by E. coli KRX expressing a panel of sulfotransferases, as well the cysP sulfate permease from B. subtilis encoded by the pCBJ-368 plasmid.

FIG. 2 pCBJ-368 plasmid used for inducible expression of the cysP gene from B. subtilis under the pT7 promoter (SEQ ID NO: 60), also including the the cysD, cysN, cysC and cysQ genes (SEQ ID NOs: 24-27).

FIG. 3A. Insertion of the sulfotransferase (pst) gene under regulation of the inducible pT7 promoter into the native E. coli Nissle 1917 (EcN) derived plasmid (pMUT) encoding the cysP, cysU, cysW, cysA (SEQ ID NOs: 20-23) under regulation of endogenous EcN promoters, and the cysD, cysN, cysC and cysQ under regulation of the pT7 inducible promoter. B. Insertion of pst gene under regulation of the constitutive Pmic7 promoter (SEQ ID NO: 61) into the native EcN derived plasmid (pMUT.3) encoding the under regulation of the endogenous promoter, cysD, cysN, cysC and cysQ.

FIG. 4A. A. Shows sulfation of EcN expressing different sulfotransferases under an inducible promoter. B. Shows sulfation of EcN expressing different sulfotransferases under a constitutive promoter. C. Shows sulfation of DCA by E. coli BL21 or E. coli KRX expressing 3 different sulfotransferases.

FIG. 5 Sulfation capacity of transporter variants integrated in the genome of EcN harboring pMUT.3-pst51. Strains were culture in biological triplicates in M9 media supplemented with 100 μM DCA (A) or 50 μM LCA (B) and 0.2% Casamino acids. One-way ANOVA (Dunnett's multiple comparison test) with 95% CI was used to determine significance, P<0.05.

FIG. 6 Identification of metabolic engineering targets for optimization of sulfation capabilities in DCA (A) and LCA (B), by using KEIO strains. Strains were cultured in biological triplicates in M9 media supplemented with either 100 μM DCA or 50 μM LCA. One-way ANOVA (Dunnett's multiple comparison test) against WT control, with 95% CI was used to determine significance, P<0.05.

FIG. 7 pETDuet-1 plasmid for expression of the pst gene under the inducible pT7 promoter.

FIG. 8 pT7pol-CHLM plasmid with T7 polymerase repressed by lacl

FIG. 9 Nucleic acid sequences of SEQ ID NOs: 20-28.

FIG. 10 Nucleic acid sequence of SEQ ID NO: 64 encoding the human SULT2A1.

FIG. 11 Extended KO targets screened for enhanced sulfation of secondary bile acids. A and B show sulfation of KEIO strains expressing SULT2A1 for DCA and LCA, respectively. Dash line represents the mean of MG1655 control. One-Way ANOVA was used to perform multiple comparison against MG1655 control.

FIG. 12 Sulfation of DCA and LCA by E. coli Nissle and S. boulardii expressing SULT2A1. Total sulfated DCA and LCA from 48- and 72-hour timepoints (A & B). Total sulfated DCA and LCA normalized per CFU of final culture (C & D).

FIG. 13 Sulfation of DCA and LCA by E. coli Nissle and S. boulardii expressing SULT2A1 under varying oxygen concentrations. A and B show Sulfation of DCA and LCA respectively by E. coli Nissle and S. boulardii expressing SULT2A1 under varying oxygen concentrations (0%, 5% and 21%).

FIG. 14 E. coli Nissle expressing SULT2A1 reduces levels of DCA and LCA in human and mouse fecal suspension matrix (FM). A shows reduction of LCA in human fecal matrix (FM) and fecal matrix with supplemented 100 μM of DCA and LCA (FM+SBAs) and incubated anaerobically (P=0.004 and P=0.0079). B shows reduction of DCA in FM supplemented with MgSO4, kanamycin and 100 μM of DCA and LCA (FM+MgSO4+KAN+SBAs) incubated anaerobically (P=0.003). C shows reduction of DCA in mouse FM supplemented with MgSO4, kanamycin and 100 μM of DCA and LCA (FM+MgSO4+KAN+SBAs) and MgSO4, kanamycin and 100 μM of DCA (FM+MgSO4+KAN+DCA) incubated aerobically (P=0.041 and P=0.0011).

FIG. 15 Sulfation of LCA and DCA by E. coli Nissle expressing SULT2A1 in human and mouse fecal suspension matrix (FM). A shows sulfation of LCA by EcN-SULFO in human FM and FM supplemented with 100 μM DCA/LCA and incubated aerobically (P=0.0004, P=0.0079). B shows sulfation of LCA by EcN-SULFO in mouse FM supplemented with 100 μM DCA/LCA and FM+MgSOP4+SBAs incubated aerobically (P=0.0001, P=0.0001). C shows sulfation of LCA by EcN-SULFO in mouse FM incubated anaerobically (P=0.0005). D shows sulfation of DCA by EcN-SULFO in different FM preparations and incubated anaerobically. Student T-test was used to compare strains.

FIG. 16 In vivo sulfation in a model host organism. About three days prior to inoculation of host with composition comprising the probiotic cell of the present invention at 10¹⁰ CFU the host is exposed to antibiotic pre-treatment. The host is inoculated with probiotic treatment every second day of the study and plasma and faeces samples are collected, before and during study. At day 4, the diet is changed to comprise 0.3% DCA or LCA for the remaining period of the study. The model host organism is sacrificed after at least 12 days post onset of treatment. Bile acid levels is evaluated multiple times during the treatment period. Additional readouts are activity, body weight and gene regulation. After study end, the model host animal is sacrificed and plasma, gut, liver and faeces are sampled. Following the study, optimally, a detection of elevated sulfation of secondary bile acids is obtained, compared to a control cohort, potentially in combination with a positive change in bile acid pools, e.g., a higher amount of sulphated secondary bile acids, such as but not limited to a higher amount of sulphated LCA and/or DCA along with derivates and conjugates thereof.

FIG. 17 In vivo sulfation in colon cancer diseases model host organism. The preventive and therapeutic potential of bile acid sulfation is optimally tested in a model host organism such as in an Apc^{min/+} (ApcMin (Min, multiple intestinal neoplasia) is a point mutation in the murine homolog of the APC gene) model for multiple intestinal adenomas. The study is a two-stage study, with the stages CRC-1 and CRC-2. In CRC-1 (early stage), three cohorts or more is tested, a first cohort treated (repeted dosing) with an advanced microbiome therapeutic (sulfo AMT) i.e., a microbiome-based therapeutic composition comprising an engineered probiotic cell expressing a sulfotransferase (Sulfo), a second non-treated control cohort and a third cohort treated (repeated dosing) with an AMT not comprising a probiotic cell of the present invention (control AMT/empty AMT). In the first stage of the study, mice are treated on a DCA diet for 12 weeks. After completion of CRC-1, CRC-2 is initiated and the control cohort (no treated with an AMT) of CRC-1 is split into three new cohorts, one cohort treated with the AMT of the present invention, one cohort receiving no treatment, and one treated with a control AMT. Following completion of CRC-2 the cohorts are dissected and the bile acid (BA) level is evaluated, and intestine tumours are scored for each cohort. Optimally, the cohort treated with the sulfo AMT has a reduced number of tumours, compared to the control groups in both CRC-1 and CRC-2. About three days prior to inoculation of host with composition comprising the probiotic cell of the present invention at 10¹⁰ CFU the host is potentially exposed to antibiotic pre-treatment. The host is inoculated with probiotic treatment every second day of the study and plasma and faeces samples are collected, before and during study. The model host organism is sacrificed after at least 12 days post onset of treatment. Bile acid levels is evaluated multiple times during the treatment period. Additional readouts are activity, body weight and gene regulation. After study end, the model host animal is sacrificed and plasma, gut, liver and faeces are sampled. qPCR is used to identify gene regulation in specific tissues.

FIG. 18 In vivo sulfation in metabolic diseases in a model host organism. About three days prior to inoculation of host with composition comprising the probiotic cell of the present invention at 10¹⁰ CFU the host is potentially exposed to antibiotic pre-treatment. The host is inoculated with probiotic treatment every second day of the study and plasma and faeces samples are collected, before and during study. The model host organism is sacrificed after at least 12 days post onset of treatment. Bile acid levels is evaluated multiple times during the treatment period. Additional readouts are activity, glucose tolerance test (GTT), body weight and gene regulation. After study end, the model host animal is sacrificed and plasma, gut, liver and faeces are sampled. qPCR is used to identify gene regulation in specific tissues.

### SEQUENCES

The genes and polynucleotides of the present invention are listed in the sequence listing, and an overview of sequences is provided in table 3.

TABLE 3
Protein and sequence listing overview

| | | | NCBI ref | SEQ |
|---|---|---|---|---|
| Protein/ | | Origin | (nucleotide sequence reference/ | ID |
| gene ID | Function | host | protein reference)* | NO: |
| SULT1A1 | Sulfotransferase | Rattus | NP_114022.1/AF394783.1 | 1, 30 |
| | | norvegicus | | |
| SULT1ST1 | Sulfotransferase | Danio | LP858384.1/AAH56729.1 | 10, 35 |
| | | rerio | | |
| SULT6B1 | Nucleic acid sequence encoding | Danio | LP858386.1 | 11 |
| | sulfotransferase SULT6B1 | rerio | | |
| SULT1A1 | Sulfotransferase | Homo | —/AAI10888.1 | 7, 31 |
| | | sapiens | | |
| SULT1A1 - | Nucleic acid sequence encoding | Rattus | — | 2 |
| clone 2 | sulfotransferase SULT1A1 - clone 2 | norvergicus | | |
| dmST1 | Sulfotransferase | Drosophila | — | 12, 36 |
| | | melanogaster | | |
| dmST1 - | Sulfotransferase | Drosophila | — | 13, 37 |
| clone2 | | melanogaster | | |
| SULT1A1 | Sulfotransferase | Equus ferus | — | 6, 34 |
| | | caballus | | |
| SULT1E1 | Sulfotransferase | Gallus gallus | —/XP_420616.4 | 14, 38 |
| | | domesticus | | |
| SULT1A1 | Sulfotransferase | Canis lupus | —/NP_001003223.1 | 4, 32 |
| | | familiaris | | |
| SULT1A1 | Sulfotransferase | Sus scrofa | — | 5, 33 |
| | | domesticus | | |
| SULT1B1- | Sulfotransferase | Gallus gallus | —/NP_001299607.1 | 15, 39 |
| predicted - | | domesticus | | |
| clone 1 | | | | |
| SULT1B1- | Sulfotransferase | Gallus gallus | — | 16, 40 |
| predicted - | | domesticus | | |
| clone2 | | | | |
| SULT1C1 | Sulfotransferase | Gallus gallus | —/NP_989932.1 | 17, 41 |
| | | domesticus | | |
| SULT1A1 | Sulfotransferase | Rattus | —/NP_114022.1 | 3, 30 |
| (codon-opt) | | norvegicus | | |
| KMZ74024.1 | Aryl sulfotransferase | Zostera | —/KMZ74024.1 | 18, 42 |
| | | marina | | |
| KMZ73756.1 | Aryl sulfotransferase | Zostera | —/KMZ73756.1 | 19, 43 |
| | | marina | | |
| SULT2A1 | sulfotransferase SULT2A1 | Homo | NM_003167.4/NP_003158.2 | 8, 29 |
| | | sapiens | | |
| SULT2A1 | Codon optimized nucleic acid | — | — | 9 |
| (codon-opt) | sequence encoding sulfotransferase | | | |
| | SULT2A1 | | | |
| cysP | Nucleic acid sequence encoding | E. coli | — | 20 |
| | Thiosulfate/sulfate ABC transporter | | | |
| | periplasmic binding protein CysP | | | |
| cysU | Nucleic acid sequence encoding | E. coli | — | 21 |
| | Sulfate/thiosulfate ABC transporter | | | |
| | inner membrane subunit CysU | | | |
| cysW | Nucleic acid sequence encoding | E. coli | — | 22 |
| | Sulfate/thiosulfate ABC transporter | | | |
| | inner membrane subunit CysW | | | |
| cysA | Nucleic acid sequence encoding | E. coli | — | 23 |
| | Sulfate/thiosulfate ABC transporter | | | |
| | ATP binding subunit CysA | | | |
| cysD | Nucleic acid sequence encoding | E. coli | — | 24 |
| | Sulfate adenylyltransferase | | | |
| | subunit 2 CysD | | | |
| cysN | Nucleic acid sequence encoding | E. coli | — | 25 |
| | Sulfate adenylyltransferase | | | |
| | subunit 1 CysN | | | |
| cysC | Nucleic acid sequence encoding | E. coli | — | 26 |
| | Adenylyl-sulfate kinase CysC | | | |
| cysQ | Nucleic acid sequence encoding | E. coli | — | 27 |
| | 3′(2′),5′-bisphosphate | | | |
| | nucleotidase CysQ | | | |
| cysP | Nucleic acid sequence encoding | Bacillus | — | 28 |
| | Sulfatepermease CysP | subtilis | | |
| CysP | Thiosulfate/sulfate ABC transporter | E. coli | NP_416920.1 | 44 |
| | periplasmic binding protein CysP | | | |
| CysU | Sulfate/thiosulfate ABC transporter | E. coli | NP_416919.1 | 45 |
| | inner membrane subunit CysU | | | |
| CysW | Sulfate/thiosulfate ABC transporter | E. coli | YP_026168.2 | 46 |
| | inner membrane subunit | | | |
| CysA | Sulfate/thiosulfate ABC transporter | E. coli | NP_416917.1 | 47 |
| | ATP binding subunit | | | |
| CysD | Sulfate adenylyltransferase subunit 2 | E. coli | NP_417232.1 | 48 |
| CysN | Sulfate adenylyltransferase subunit 1 | E. coli | NP_417231.1 | 49 |
| CysC | Adenylyl-sulfate kinase | E. coli | NP_417230.1 | 50 |
| CysQ | 3′(2′),5′-bisphosphate nucleotidase | E. coli | NP_418635.1 | 51 |
| CysP | Sulfatepermease | E. coli | NP_389441.1 | 52 |
| yjcS | Nucleic acid sequence encoding the | E. coli | NC_000913.3:4306597-4304612 | 53 |
| | SDS sulfatase YjcS | | | |
| aslA | Nucleic acid sequence encoding the | E. coli | NC_000913.3:3986007-3984352 | 54 |
| | putative Ser-type sulfatase AslA | | | |
| ydeN | Nucleic acid sequence encoding the | E. coli | NC_000913.3:1582524-1580842 | 55 |
| | putative Ser-type sulfatase YdeN | | | |
| yidJ | Nucleic acid sequence encoding the | E. coli | NC_000913.3:3858404-3856911 | 56 |
| | putative Cys-type sulfatase YidJ | | | |
| ydeM | Nucleic acid sequence encoding the | E. coli | NC_000913.3:1580790-1579633 | 57 |
| | anaerobic sulfatase maturation | | | |
| | enzyme YdeM | | | |
| aslB | Nucleic acid sequence encoding the | E. coli | NC_000913.3:3982958-3984193 | 58 |
| | anaerobic sulfatase maturation | | | |
| | enzyme AslB | | | |
| hdhA | Nucleic acid sequence encoding the | E. coli | NC_000913.3:1698040-1697273 | 59 |
| | 7-α-hydroxysteroid dehydrogenase | | | |
| | HdhA | | | |
| T7 | Inducible promoter sequence | — | — | 60 |
| Pmic7 | Constitutive promoter sequence | — | — | 61 |
| BBa_J23110 | promoter sequence | — | — | 62 |
| pMUT REV | pMUT backbone reverse primer | — | — | 63 |
| Primer | | | | |
| SULT2A1 | S. boulardii codon optimized | — | — | 64 |
| (Opt) | SULT2A1 | | | |
| SULT2A1-fw | S. boulardii SULT2A1-fw primer | — | — | 65 |
| primer | | | | |
| SULT2A1 rv | S. boulardii SULT2A1-rv primer | — | — | 66 |
| primer | | | | |
| pCfB2055-fw | S. boulardii pCfB2055-fw primer | — | — | 67 |
| primer | | | | |
| pCfB2055 rv | S. boulardii pCfB2055-rv primer | — | — | 68 |
| primer | | | | |
| cysH | Nucleic acid sequence encoding the | E. coli | ECK2757 | 69 |
| | phosphoadenosine phosphosulfate | | | |
| | reductase CysH | | | |
| cysQ | Nucleic acid sequence encoding the | E. coli | ECK4210 | 70 |
| | 3′(2′),5′-bisphosphate nucleotidase | | | |
| | CysQ | | | |
| acrB | Nucleic acid sequence encoding the | E. coli | ECK0456 | 71 |
| | multidrug efflux pump RND | | | |
| | permease AcrB | | | |
| ssuB | Nucleic acid sequence encoding the | E. coli | ECK0924 | 72 |
| | aliphatic sulfonate ABC transporter | | | |
| | ATP binding subunit SsuB | | | |
| emrB | Nucleic acid sequence encoding the | E. coli | ECK2680 | 73 |
| | multidrug efflux pump membrane | | | |
| | subunit EmrB | | | |
| pstl | Nucleic acid sequence encoding the | E. coli | ECK2411 | 74 |
| | phosphoenolpyruvate-protein | | | |
| | phosphotransferase Pstl | | | |

*Protein are referred to with a protein reference (https://www.ncbi.nlm.nih.gov/protein/) and genes are referred with either the genome reference (https://www.ncbi.nlm.nih.gov/nuccore), where NC_ number refers to the host reference sequence, and the remaining part refers to the gene position in the reference sequence or to the gene reference (https://www.ncbi.nlm.nih.gov/gene/).

### EXAMPLES

It should be understood that any feature and/or aspect discussed above in connections with the compounds according to the invention apply by analogy to the methods described herein. The following figures and examples are provided below to illustrate the present invention. They are intended to be illustrative and are not to be construed as limiting in any way.

### Example 1—Sulfotransferase Screen for Sulfation of Secondary Bile Acids in E. coli KRX.

Sulfotransferase Screen

Example 1 is used to demonstrate that expression of a sulfotransferase in a host cell, can lead to sulfation of secondary bile acids in vitro. In total 43 different sulfotransferases were tested for their ability to sulfate secondary bile acids.

Material and Methods

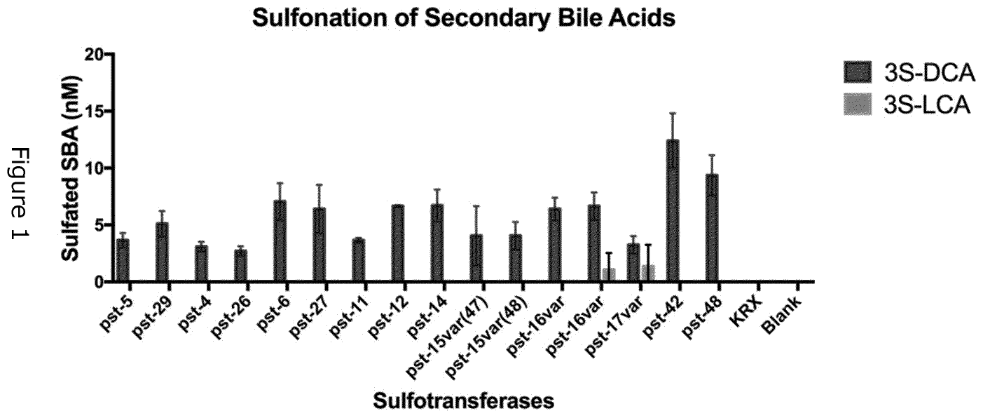

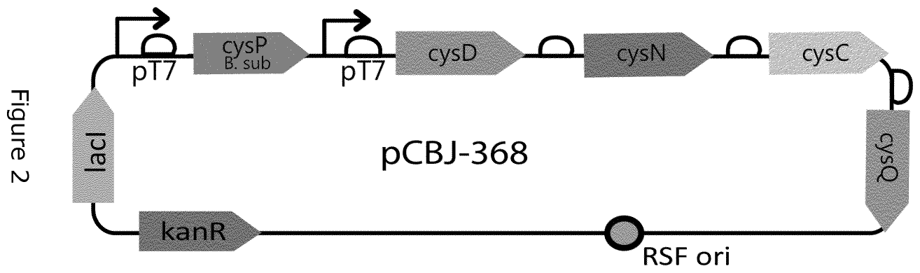

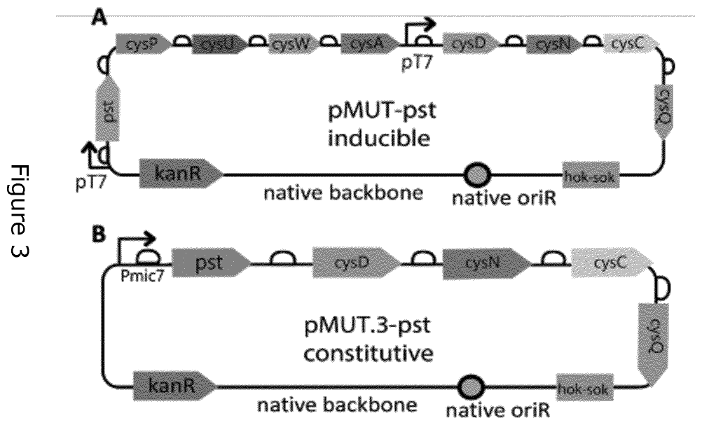

A library of 43 sulfotransferases, as listed in table 1, were expressed and tested as described below, for in vitro inducible sulfation using E. coli KRX (FIG. 1). The E. coli KRX strains harbouring the sulfotransferase plasmid and pCBJ368 (FIG. 2) were inoculated into 350 μl of 2xYT, supplemented with the appropriate antibiotics, using a 96-deepwell plate. The plate was incubated at 37° C. for 24 hours in a shaking incubator. After 24 hours, the culture was diluted 1:100 into M9 minimal media (See table 4 and table 5) including a final of 0.4% glucose and supplemented with 50 μM LCA or 100 M DCA. Plate was incubated for 24 additional hours, before harvesting the supernatant by centrifugation at 4500 g for 10 minutes. Samples were then frozen at −20° C. until further processing for LC-MS/MS analysis, which was conducted using 50 μl of bacterial broth was extracted with 3 volumes of methanol containing deuterated internal standards (d4-DCA or d4-LCA; 50 nM of each). After 10 minutes of vortex and 10 minutes of centrifugation at 20 000 g, the supernatant was diluted 1:50 in methanol:water [1:1]. Bile acids were analyzed using ultra-performance liquid chromatography-tandem mass spectrometry (UPLC-MS/MS) which was done by injection of (5 μL) the bile acids which were afterwards separated on a C18 column (1.7u, 2.1×100 mm; Kinetex, Phenomenex, USA) using water with 7.5 mM ammonium acetate and 0.019% formic acid (mobile phase A) and acetonitrile with 0.1% formic acid (mobile phase B). The chromatographic separation started with isocratic separation at 20% B for 1 minute. The B-phase was then increased to 35% for 4 minutes. During the next 10 minutes the B-phase was increased to 100%. The B-phase was held at 100% for 3.5 minutes before returning to 20%. The total runtime was 20 minutes. Bile acids were detected using multiple reaction monitoring (MRM) in negative mode on a QTRAP 5500 mass spectrometer (Sciex, Concord, Canada).

TABLE 4
M9 media composition

| Stock Solution/ | | |
|---|---|---|
| Compounds | Per 1 Liter | |
| MQ H₂O | Up to 1 L | |
| 20% (w/v) glucose | 20 ml for 0.4% | |
| 10X M9 salts | 100 | ml |
| 2M MgSO₄ | 1 | ml |
| Trace elements | 500 | μl |
| (table 5) | | |
| Wolfe's vitamin | 1 | ml |
| 1M CaCl₂ | 100 | μl |

TABLE 5
10x M9 salts

| Compound | Amount per 1 L | |
|---|---|---|
| Disodium EDTA | 15 | g |
| ZnSO₄ 7H₂O | 4.5 | g |
| MnCl₂ 4H₂O | 0.7 | g |
| CoCl₂ 6H₂O | 0.3 | g |
| CuSO₄ 2H₂O | 0.2 | g |
| Na₂MoO₄ 2H₂O | 0.4 | g |
| CaCl₂ 2H₂O | 4.5 | g |
| FeSO₄ 7H₂O | 3 | g |
| H₃BO₃ | 1 | g |
| Kl | 0.1 | g |

Sult2a1op (pst-51) was codon optimized by Integrated DNA technologies (IDT) internal algorithms, codon optimized sulfotransferase DNA fragments were subsequently ordered from IDT.

Results

The screen showed that some sulfotransferases were capable of sulfating the secondary bile acids, LCA and DCA, as is shown in FIG. 1.

### Example 2—Inducible and Constitutive Sulfation of Secondary Bile Acids in E. coli Nissle, BL21 or KRX Expressing Different Sulfotransferases

Expression of a Sulfotransferase in Different E. coli Strains

Example 2 describes the expression of a sulfotransferase in the different E. coli strains KRX, BL21 and Nissle 1917, furthermore, this example describes the regulation of the expression, using either an inducible promoter or a constitutive promoter. Also, this example describes combination of the sulfotransferase gene, with the Cys permease genes cysP, cysU, cysW, cysA and/or the sulfate recycling related genes cysD, cysN, cysC and cysQ.

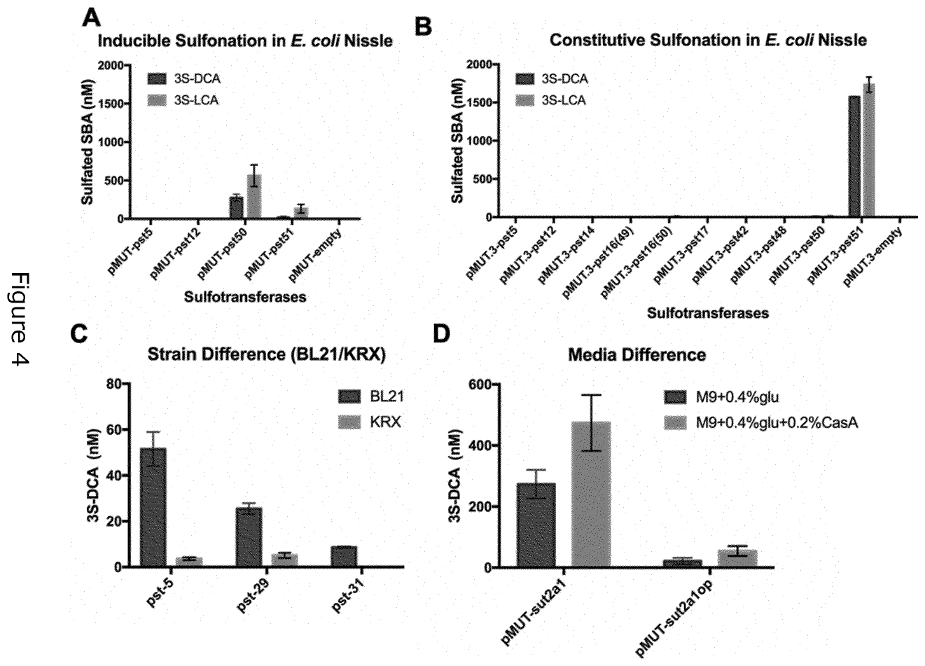

Sulfation activity was tested in EcN, obtained from commercially available Mutaflor product, using an inducible system (FIG. 3A) and a constitutive system (FIG. 3B) but cloned into a native EcN plasmid of the strain, and incorporating the sulfotransferases, pst50 (SEQ ID NO: 8) or pst51 (SEQ ID NO: 9), which encode for the native and codon optimized human SULT2A1 gene, both encoding the expression of the amino acid sequence of SEQ ID NO: 29.

Materials and Methods

Cloning reaction was performed using 1 μl of USER enzyme (NEB), 1 μl of Dpnl (ThermoFisher Scientific), 1 μl of 10X Cut Smart buffer (NEB) and 200 ng of DNA fragments and MQ water, for a total reaction volume of 10 μl. Mixture was incubated at 37° C. for 30 minutes, followed by 15 minutes at 15° C. 5 μl of the USER reaction was used to transformed chemically competent E. coli TOP10 (Invitrogen, Carlsbad, CA, USA) by heat-shock at 42° C. 1 ml of SOC media was used to recover the transformed cells for 1 hour, and 50 μl were plated in LB plates supplemented with the appropriate antibiotic. Plates were incubated at 37° C. overnight. Next day colonies were screened through colony PCR, using OneTaq Quick-Load 2x Master Mix (NEB). Positive colonies were inoculated into 5 ml of 2xYT medium (containing 16 g/L Tryptone, 10 g/L Yeast Extract and 5 g/L NaCl) and were incubated overnight. Next day, plasmids were purified using NucleoSpin Plasmid EasyPure purification kit (Macherey-Nagel), following manufacturer's instructions. Concentration of purified plasmids were measured with NanoDrop (ThermoScientific), and later sequenced using Eurofins overnight sequencing service. Cultures of colonies harboring the correct plasmid were stored at −80° C. as glycerol stocks.

A single colony of EcN was inoculated in LB media overnight. Next day 100 μl of the overnight culture was used to inoculate 10 ml of 2xYT. Optical density (OD) was followed, and cultures were harvested between OD600=0.4-0.5, using a prechilled centrifuged at 4500 g for 10 minutes. Pellets were washed 3 times with cold 10% glycerol/MQ H₂O solution. Lastly, 100 ng of the desired plasmid was transferred onto the pellets and 50 μl of 10% glycerol/MQ H2O solution was used for resuspension. Resuspended cells were then transferred to a cold 0.1 cm Gene Pulser electroporation cuvette (Bio-Rad) and were electroporate (BioRad MicroPulser) at 1.8 kV. Cells were recovered using 1 ml of SOC media for 1 hour in a shaking incubator at 37° C., before plating.

In order to prepare the bacteria for sulfation experiments small scale fermentations were performed by inoculating strains, in biological duplicates, unless otherwise stated, into 500 μl of M9 media (table 4 and table 5) (0.4% glucose) supplemented with appropriate antibiotics in 96-deep well plates. Preculture was allowed to grow until saturation (24 hours), after which an aliquot of 5 μl was taken to inoculate the production culture (500 μl), using the same setup. After 22 hours, optical density was measured, and plates were centrifuged at 4500 rpm for 10 min. Supernatants were then frozen until further LC-MS/MS preparations were performed. For fermentations of KRX strains, 2xYT was used for preculture, and rhamnose (0.1%) and IPTG (0.1 mM) was added to the production culture to induce expression T7 RNA polymerase.

Plasmids (FIG. 3A and 3B) were constructed following standard cloning methods, as described above. Fragments of interest containing the sulfotransferase gene, the genes encoding for the recycling genes, cysD, cysN, cysC and cysQ and the sulfate transporters, cysP, cysU, cysW cysA from E. coli or, were amplified and cloned using USER cloning, as a single operon. Promoters were incorporated in the reverse primer (SEQ ID NO: 63) used for amplifying the backbone fragment from the pMUT plasmid. For the inducible approach, the T7 promoter (SEQ ID NO: 60) was used to drive expression of the sulfotransferase, recycling enzymes and the transporters. Additionally, a plasmid having the T7 polymerase repressed by lacl had to be transformed in the strains harboring the inducible sulfation system (FIG. 8). A strong constitutive promoter, Pmic7, (SEQ ID NO: 61), was used for driving expression of the constitutive sulfation approach (FIG. 3B).

Results

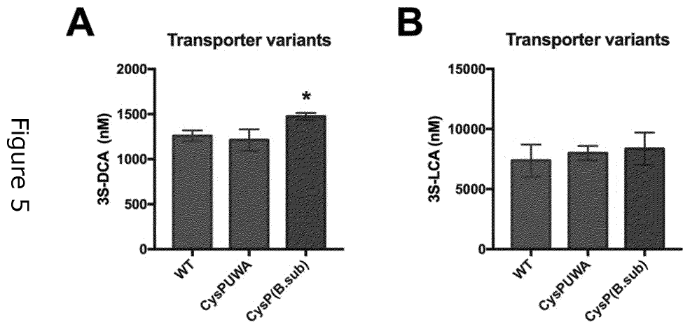

The human SULT2A1 variants pst-50 and pst-51 showed to be the only ones having activity in this setup (FIG. 4A). The non-codon optimized version outperformed the optimized version for both DCA and LCA sulfation. This clearly provides a proof of concept, that expression of a specific sulfotransferase, not just any sulfotransferase, can be used to solve the problem of the invention, of enhancing the levels of sulfated secondary bile acids.

In the constitutive system, having both the sulfotransferase and the sulfate recycling related genes cysD, cysN, cysC and cysQ driven by a strong constitutive promoter, the codon optimized SULT2A1, pst51, showed much greater activity towards DCA and LCA than the non-optimized variant (FIG. 4B), reaching similar sulfation values for both secondary bile acids.

The combination of having a strong promoter to promote expression of the codon optimized SULT2A1 in the probiotic cell, and a strong promoter promoting expression of the sulfate recycling related genes, cysD, cysN, cysC and cysQ in the probiotic cell, clearly shows that combining the expression of the sulfotransferase and the cofactor recycling genes, can enhance the level of sulfation of secondary bile acids.

The sulfation activity of E. coli BL21 (obtained from ThermoScientific) and E. coli KRX (obtained from Promega) following transformation with the plasmid according to FIG. 3A, encoding pst-5 (SEQ ID NO: 1), pst-29 (SEQ ID NO: 10) or pst-31 (SEQ ID NO: 11), resulted showed LCA and DCA sulfation, as shown in FIG. 4C. The level of sulfation obtained in E. coli KRX and E. coli BL-21 were low compared to the level of sulfation obtainable in EcN (FIG. 4A and FIG. 4B), also indicating that the specific choice of E. coli also has some effect on the level of sulfation. For pst-5 no sulfation was observed in EcN while a small amount of was seen for E. coli KRX and E. coli BL-21.

Example 2 teaches that taking a sulfotransferase that have been seen to work in one E. coli strain and transform it into a different strain might not be successful. Thus, it is not possible to select any sulfotransferase and any host cell and obtain sulfation of secondary bile acids as a result. Example 2 clearly states that combination the sulfotransferase gene with the transporter genes cysP, cysU, cysW cysA and/or the sulfate recycling related genes cysD, cysN, cysC and cysQ, with the right promoter sequences is essential in obtaining sulfation of secondary bile acids.

Example 3—Upregulation of a Sulfate Transporter

Regulation of Expression

Example 3 describes the upregulation of the endogenous sulfate uptake machinery of E. coli, and the expression of the sulfate permease, cysP from B. subtilis, which can also be used to drive sulfation of secondary bile acids.

Materials and Methods

Plasmids (FIG. 3A and 3B) were constructed following standard cloning methods, as described above. Fragments of interest containing the sulfotransferase gene, the genes encoding for the sulfate recycling related genes, cysD, cysN, cysC and cysQ and the sulfate permease gene cysP from B. subtilis (SEQ ID NO:28), were amplified and cloned using USER cloning, as a single operon. Promoters were incorporated in the reverse primer (SEQ ID NO: 63) used for amplifying the backbone fragment from the pMUT plasmid. For the inducible approach, the T7 promoter (SEQ ID NO: 60) was used to drive expression of the sulfotransferase, recycling enzymes and the transporters. Additionally, a plasmid having the T7 polymerase repressed by lacl had to be transformed in the strains harboring the inducible sulfation system (FIG. 8). A strong constitutive promoter, Pmic7, (SEQ ID NO: 61), was used for driving expression of the constitutive sulfation approach (FIG. 3B).

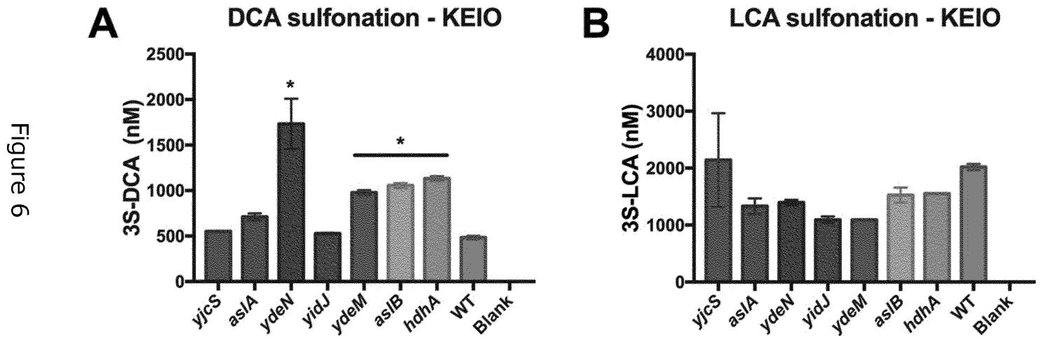

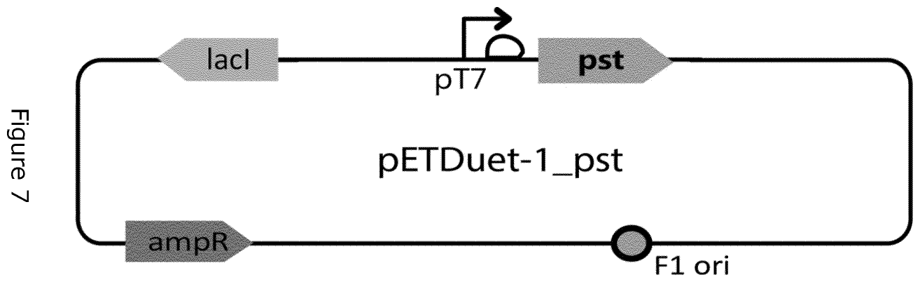

Improved sulfate uptake capabilities were tested in order to assess whether the native uptake system was a limitation for optimal secondary bile acid sulfation. Integration of a low-mid strength Anderson promoter (BBa_J23110, SEQ ID NO: 63) substituting the native promoter, upstream of the cysP, cysU, cysW and cysA genes, which forms part of E. coli's native sulfate/thiosulfate uptake machinery, was introduced. Another approach tested consisted of boosting native uptake capabilities of the cysP, cysU, cysW and cysA genes, by integrating a copy of cysP gene from B. subtilis, which is part of the inorganic phosphate transporter family and that has been shown to restore sulfate starvation. When tested together with the constitutive expression of pst51 and cysD, cysN, cysC and cysQ, only cysP from B. subtilis seemed to improve significantly sulfation of DCA (FIG. 5A). LCA, on the other hand, showed no significant improvements (FIG. 5B).

Results

Example 3 clearly shows that sulfation of secondary bile acids can be enhanced by introduction of cysP from B. subtilis into EcN. It would on the basis of this example 1-3, be obvious to combine the codon optimized human SULT2A1 under a constitutive promoter (SEQ ID NO: 61), with the cysP gene from B. subtilis, under a constitutive promoter (SEQ ID NO: 61) in a single construct, for insertion into a plasmid or for genomic integration.

### Example 4—Sulfation Related Genes which Enhance or Decrease Sulfation of Secondary Bile Acids

Knockout of Sulfate Related Genes

Example 4 describes knockout (KO) of specific genes, that affect the level of sulfation of secondary bile acids.

Materials and Methods

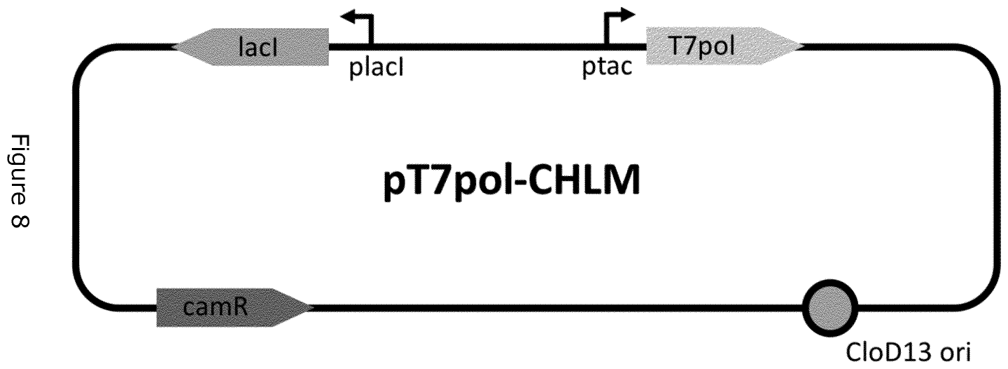

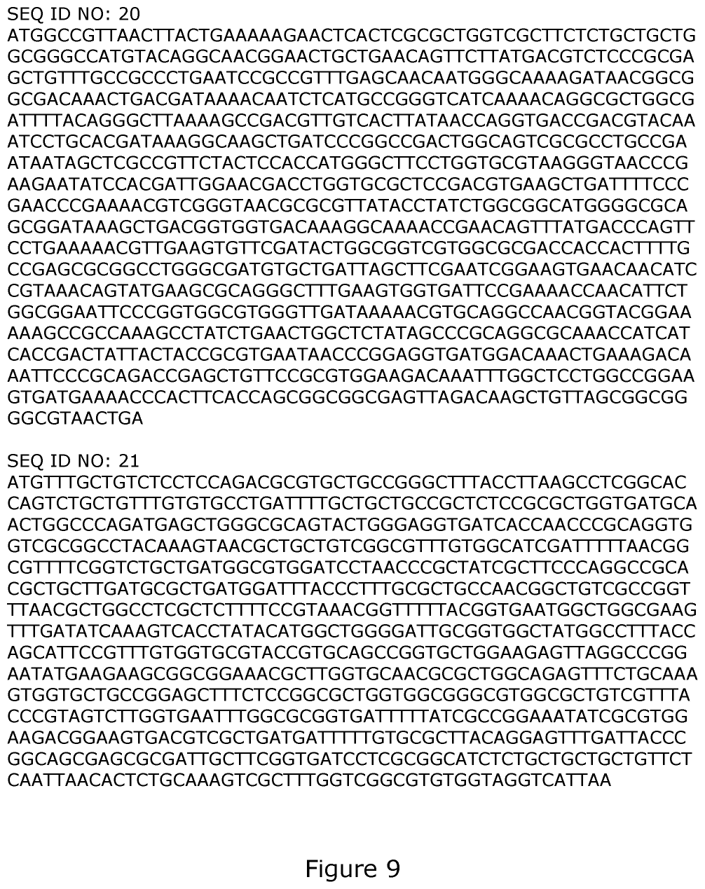

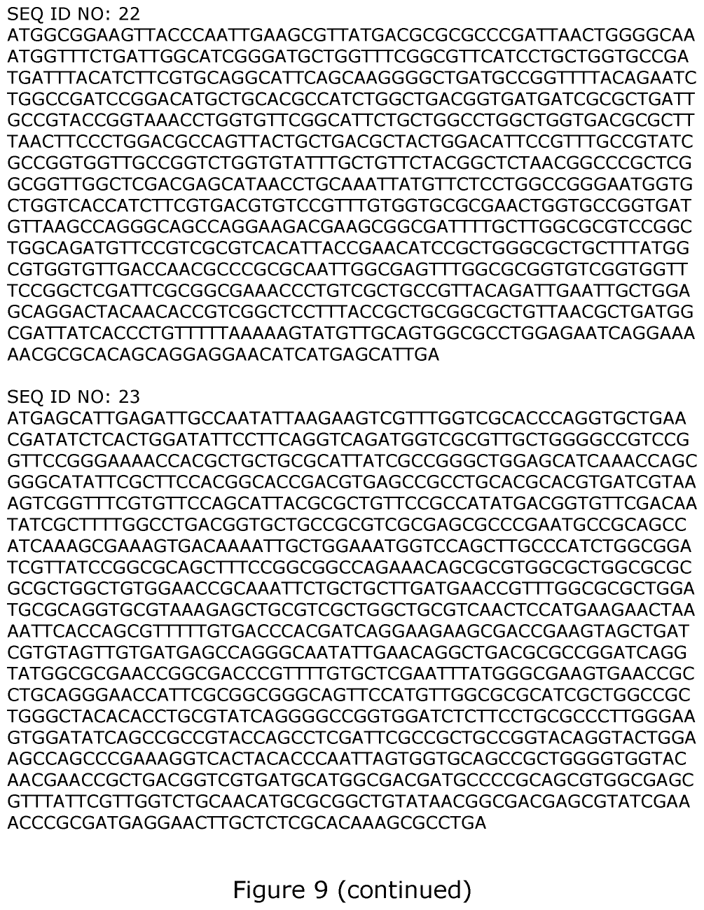

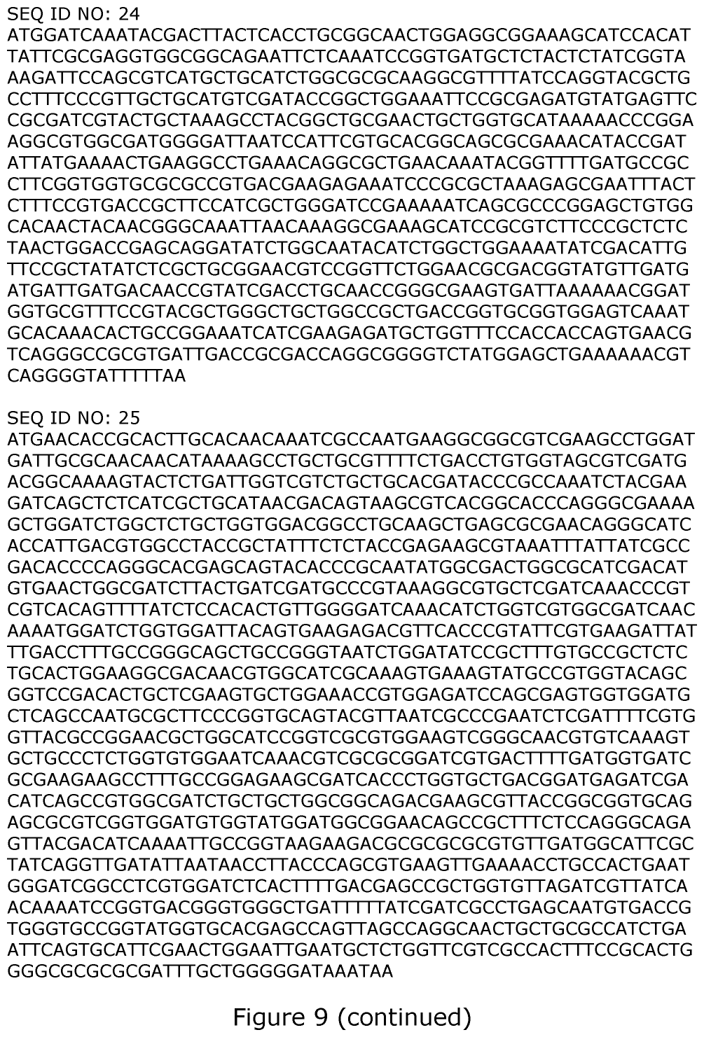

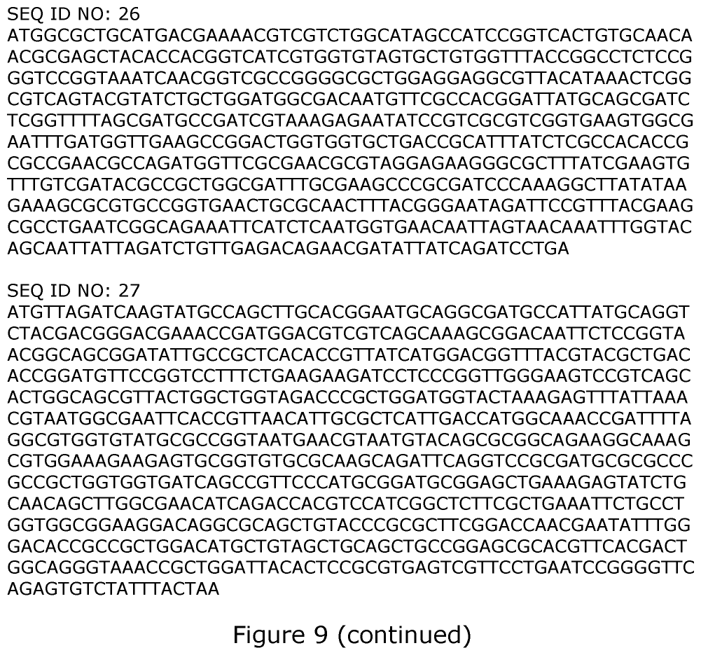

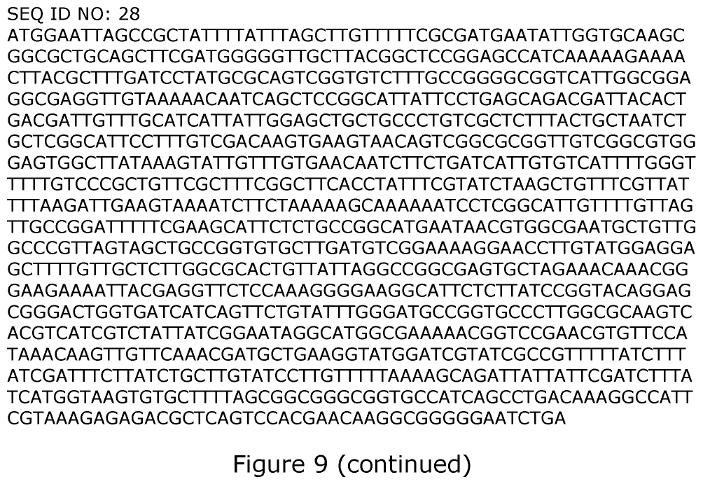

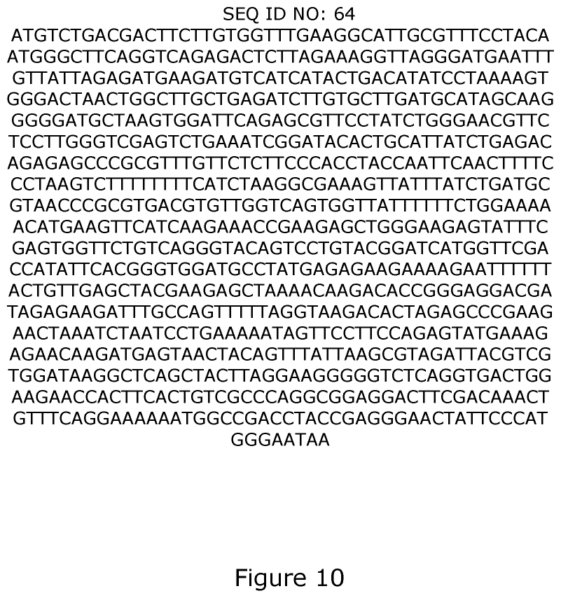

In order to identify metabolic engineering targets for increasing sulfation capabilities in E. coli, a plasmid encoding the pst50 gene, human SULT2A1 non-codon optimized, was transformed into several KEIO strains (FIG. 6A and FIG. 6B). The KEIO collection is a publicly available collection of single gene KOs in E. coli K-12. These strains had KOs for known and putative sulfatases, and sulfatase maturating enzymes (table 6). One-way ANOVA analysis showed that KO of ydeN, ydeM, asIB and hdhA significantly increased the sulfation of DCA (FIG. 6A).

TABLE 6
KEIO strain KO genes

| Gene | Function |
|---|---|
| yjcS | SDS sulfatase |
| aslA | putative Ser-type sulfatase |
| ydeN | putative Ser-type sulfatase |
| yidJ | YidJ is a putative Cys-type sulfatase |
| ydeM | YdeM, member of the anaerobic sulfatase |
| | maturation enzyme subfamily of the |
| | Radical SAM superfamily of enzymes |
| aslB | AslB is a member of the anaerobic sulfatase |
| | maturation enzyme subfamily of the |
| | Radical SAM superfamily of enzymes |
| hdhA | 7-α-hydroxysteroid dehydrogenase catalyzes |
| | the dehydroxylation of cholic and |
| | chenodeoxycholic acids |

A single colony of each KEIO strain was inoculated in LB media overnight. Next day 100 μl of the overnight culture was used to inoculate 10 ml of 2xYT. Optical density (OD) was followed, and cultures were harvested between OD600=0.4-0.5, using a prechilled centrifuged at 4500 g for 10 minutes. Pellets were washed 3 times with cold 10% glycerol/MQ H₂O solution. Lastly, 100 ng of the desired plasmid was transferred onto the pellets and 50 μl of 10% glycerol/MQ H2O solution was used for resuspension. Resuspended cells were then transferred to a cold 0.1 cm Gene Pulser electroporation cuvette (Bio-Rad) and were electroporate (BioRad MicroPulser) at 1.8 kV. Cells were recovered using 1 ml of SOC media for 1 hour in a shaking incubator at 37° C., before plating.

In order to prepare the bacteria for sulfation experiments small scale fermentations were performed by inoculating KEIO strains (previously cured from genomic kanamycin marker), in biological duplicates, unless otherwise stated, into 500 μl of M9 media (table 4 and table 5) (0.4% glucose) supplemented with appropriate antibiotics in 96-deep well plates. Preculture was allowed to grow until saturation (24 hours), after which an aliquot of 5 μl was taken to inoculate the production culture (500 μl), using the same setup. After 22 hours, optical density was measured, and plates were centrifuged at 4500 rpm for 10 min. Supernatants were then frozen until further LC-MS/MS preparations were performed. Quantification of sulfated bile acids was conducted as described in Example 1.

Results

None of the KOs showed to decrease sulfation, compared to a E. coli MG1655 WT control (FIG. 6A). LCA sulfation was largely decreased by the presence of the KOs, with the exception of yjcS (FIG. 6B). LCA sulfation was significantly decreased by yidJ and ydeM KO strains. E. coli MG1655 is not to be considered the background strain of the KEIO collection. The KEIO parent strain have the genotype, lacl⁺rrnB_{T14} ΔlacZ_{WJ16} hsdR514 ΔaraBAD_{AH33} ΔrhaBAD_{LD78} rph-1 Δ(araB-D)567 Δ(rhaD-B)568 ΔlacZ4787(::rrnB-3) hsdR514 rph-1, while the MG1655 have the genotype K-12 F⁻ λ⁻ ilvG⁻ rfb-50 rph-1. E. coli MG1655 is considered an E. coli K-12 WT strain in many regards and has been maintained as a laboratory strain with minimal genetic manipulation, having only been cured of the temperate bacteriophage lambda and F plasmid by means of ultraviolet light and acridine orange.

Sulfation performance in KEIO strains (FIG. 6A and FIG. 6B) clearly show a positive effect of knocking out sulfatase maturation units ydeM and asIB, and ydeN, a gene encoding for a putative sulfatase (table 5). Anaerobic sulfatase maturating enzymes (YdeM and AsIB) also seems to play a role on aerobic sulfatase capabilities. Knockout of hdhA (table 2) increases sulfation of DCA (FIG. 6A).

Example 4 teaches that knockout of one or more genes related to sulfation of secondary bile acids can have a beneficial effect on the level of sulfation. On the basis of examples 1-4, it would be obvious to combine the codon optimized human SUL2A1 under a constitutive promoter (SEQ ID NO: 61), with the cysP gene from B. subtilis, under a constitutive promoter (SEQ ID NO: 61) in a single construct, for insertion into a plasmid or for genomic integration, while also knocking out (KO) one or more of the genes which have a beneficial effect on the level of sulfation, thus generating a microbiome-based therapeutic capable of sulfating secondary bile acids and xenobiotics.

### Example 5—Additional Sulfation Related Genes which Enhance or Decrease Sulfation of Secondary Bile Acids when Knocked Out

Materials and Methods

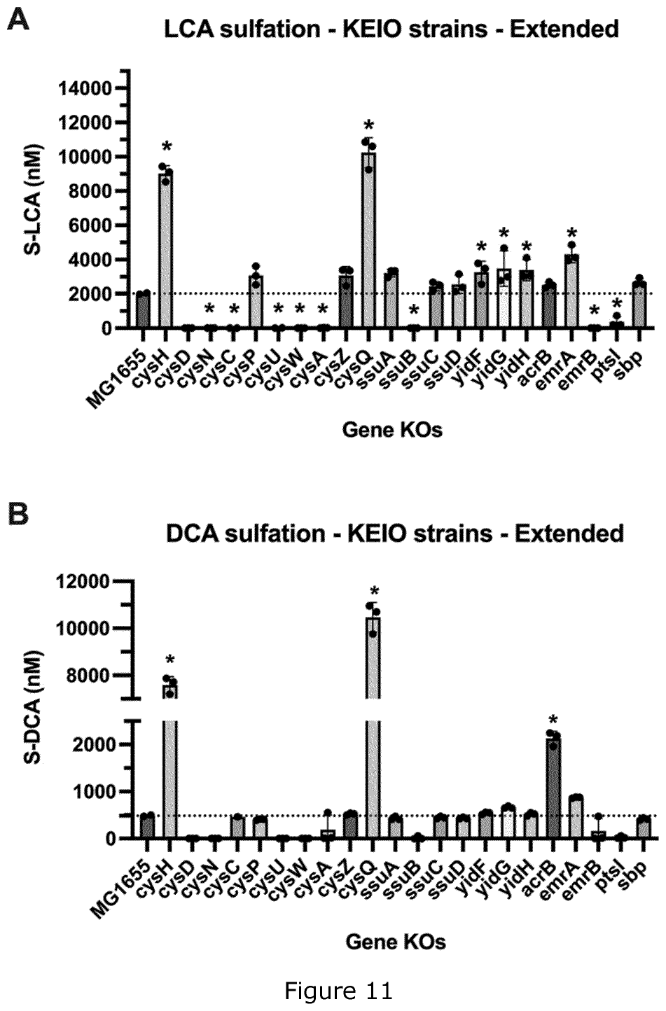

In order to identify metabolic engineering targets for increasing sulfation capabilities in E. coli, a plasmid encoding the human SULT2A1 non-codon optimized, was transformed into several KEIO strains (FIG. 11). The KEIO collection is a publicly available collection of single gene KOs in E. coli K-12. These strains had KOs for known and putative sulfatases, and sulfatase maturating enzymes (table 2). One-way ANOVA analysis showed that KO cysH and cysQ significantly increased the sulfation of DCA and LCA (FIG. 11), while KO of yidF, yidG, yidH and emrA only significantly increased LCA sulfation, and acrB only significantly increased DCA sulfation. A single colony of each KEIO strain was inoculated in LB media overnight. Next day 100 μl of the overnight culture was used to inoculate 10 ml of 2xYT. Optical density (OD) was followed, and cultures were harvested between OD600=0.4-0.5, using a prechilled centrifuged at 4500 g for 10 minutes. Pellets were washed 3 times with cold 10% glycerol/MQ H₂O solution. Lastly, 100 ng of the desired plasmid was transferred onto the pellets and 50 μl of 10% glycerol/MQ H2O solution was used for resuspension. Resuspended cells were then transferred to a cold 0.1 cm Gene Pulser electroporation cuvette (Bio-Rad) and were electroporate (BioRad MicroPulser) at 1.8 kV. Cells were recovered using 1 ml of SOC media for 1 hour in a shaking incubator at 37° C., before plating.

In order to prepare the bacteria for sulfation experiments small scale fermentations were performed by inoculating KEIO strains (previously cured from genomic kanamycin marker), in biological duplicates, unless otherwise stated, into 500 μl of M9 media (table 3 and table 4) (0.4% glucose) supplemented with appropriate antibiotics in 96-deep well plates. Preculture was allowed to grow until saturation (24 hours), after which an aliquot of 5 μl was taken to inoculate the production culture (500 μl), using the same setup. After 22 hours, optical density was measured, and plates were centrifuged at 4500 rpm for 10 min. Supernatants were then frozen until further LC-MS/MS preparations were performed. Quantification of sulfated bile acids was conducted as described in example 1.

### Example 6—Sulfation of DCA and LCA by E. coli Nissle and S. boulardii

Plasmid Construction for S. boulardii

The oligonucleotides and gBlock sequences were codon-optimised and ordered from Integrated DNA Technologies, IDTs listed in table 7. All plasmids for S. boulardii were constructed using Gibson Assembly Master Mix (Gibson et al., 2009; New England Biolabs) and are listed in table 8. Phusion high-fidelity DNA polymerase (Thermo Scientific, Waltham, MA, USA) was used for amplifying SULT2A1. SULT2A1 was assembly with the 2μ plasmid pCfB0132. The assembly reactions were used to transform competent One Shot® TOP10 Escherichia coli (Thermo Fisher Scientific, Waltham, MA, USA) cells and extracted with GeneJET Plasmid Miniprep Kit (Thermo Scientific, Waltham, MA, USA) and verified with sequencing. All E. coli cultures were grown in lysogeny broth (LB) media containing 5 g/L yeast extract, 10 g/L tryptone and 10 g/L NaCl; (Sigma Aldrich-Merck Life Science) supplemented with 100 mg/L ampicillin. For LB ampicillin agar plates, 16 g/L agar was added.

TABLE 7
S. boulardii primers

| Oligo | | |
|---|---|---|
| Name | Sequence | SEQ ID NO |
| SULT2A1- | TCGTCATCCTTGTAATCCATCGATACTAGTcaacggaatgc | 65 |
| fw | gtgcgatcg | |
| SULT2A1- | CTAACTCCTTCCTTTTCGGTTAGAGCGGAT | 66 |
| rv | | |
| pCfB2055- | ATCCGCTCTAACCGAAAAGGAAG | 67 |
| fw | | |
| pCfB2055- | GAATGCACGCGATCGCAC | 68 |
| rv | | |

TABLE 8
S. boulardii Plasmids

| | | Antibiotic resistance |
|---|---|---|
| Plasmid | Genotype | marker |
| pCfB353 | X-2-loxP-KanMX4-loxP | kanMX |
| pCfB2055 | X-2 loxP-KanMX4-loxP | kanMX |
| pCfB2055- | pCfB2055; TEF1p- | kanMX |
| GFP | GFP-CYC1t | |
| pCfB0132 | TEF1p-CYC1t | URA |
| pCfB0132- | pCfB0132; TEF1p- | URA |
| SULT2A1 | SULT2A1-CYC1 | |

Strain Construction

All S. boulardii and E. coli strains used in this study are listed in table 9.

S. boulardii transformations were performed via high-efficiency yeast transformation using the LiAc/SS carrier DNA/PEG method (Gietz et al., 2006). S. boulardii carrying pCfB2055-GFP was selected on YPD agar plates (10 g/L yeast extract, 20 g/L casein peptone, 20 g/L agar and 20 g/L glucose) containing 200 mg/L geneticin (G418; Sigma Aldrich—Merck Life Science). For selection for auxotroph markers in S. boulardii (URA3), synthetic complete (SC) dropout medium was used (6.7 g/L yeast nitrogen base without amino acids, 0.77 g/L of Complete Supplement Mixture (CSM) (Sigma-Aldrich, St. Louis, MO, USA) without uracil, 20 g/L agar and 20 g/L glucose).

TABLE 9
strain genotypes

| | | Parental |
|---|---|---|
| Strain | Genotype | strain |
| EcN-S | EcN(Tn7::CysP(B.sub) + | EcN* |
| | pMUT-SULFO.3-SULTop | |
| EcN-C | EcN(Tn7::CysP(B.sub) + | EcN* |
| | pMUT-SULFO.3-Empty | |
| SbU | ΔURA3 | SB**-ATCC-796 |
| SbU-GFP | X-2 loxP-KanMX4-loxP | SbU |
| | TEF1p-GFP-CYC1t | |
| SbU- | X-2 loxP-KanMX4-loxP | SbU-GFP |
| SULT2A1 | TEF1p-GFP-CYC1t + | |
| | pCfB0132 TEF1p- | |
| | SULT2A1-CYC1t | |

*EcN: E. coli Nissle 1917
**SB: S. boulardii

Sulfation Assessment in E. coli Nissle and S. boulardii

S. boulardii and E. coli Nissle were cultivated in DELFT minimal medium containing 7.5 g/L (NH4)2SO4, 14.4 g/L KH2PO4, 0.5 g/L MgSO4·7H20, 20 g/L glucose, 2 mL/L trace metals solution, and 1 mL/L vitamins, supplemented with 50 μM LCA or 100 μM DCA. The pH was adjusted to 6. Liquid cultures were performed in biological triplicates aerobically at 37° C. in a 24 deep well plates with a shaking of 250 rounds per minute (RPM) and with an initial OD600 of 0.10. Cultures were harvested after 48 h and 72 h, centrifuged at 10,000 g and supernatant were collected and stored at −20 ° C.

Sulfation Assessment in E. coli Nissle and S. boulardii Under GI Tract Mimicking Conditions

Cultivations followed the same protocol as above. For 5% oxygen condition, a Biotek Synergy H1 couple with gas-controlled mechanism was used. For 0% oxygen, plate was incubated in an anaerobic container with anaerobic atmosphere generation bags. Same strains as above. After 24 hours of incubation, cultures were centrifuged at 5000 G for 10 minutes, supernatant was taken and stored at −20° C. until processing for LC-MS/MS. Analytics were performed as previously described.

Results

Quantification of sulfated secondary bile acids from supernatant of small-scale fermentations. Both E. coli Nissle 1917 and S. boulardii can sulfate LCA and DCA by expressing human a codon optimized sulfotransferase SULT2A1. E. coli Nissle seems to produce more sulfated DCA and approximately the same amount of sulfated LCA, however, when normalized per CFU of culture S. boulardii outperforms E. coli Nissle 1917 (FIG. 12).

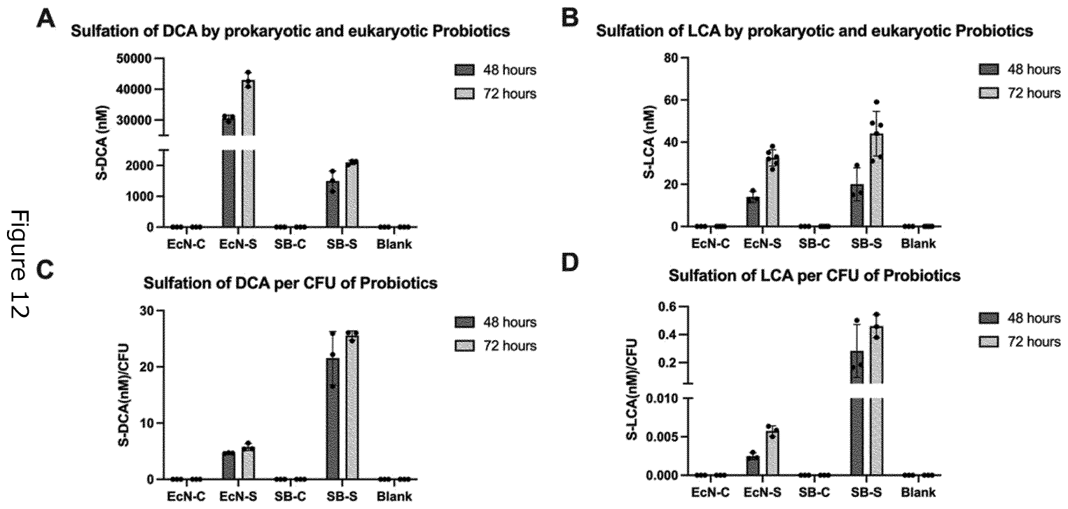

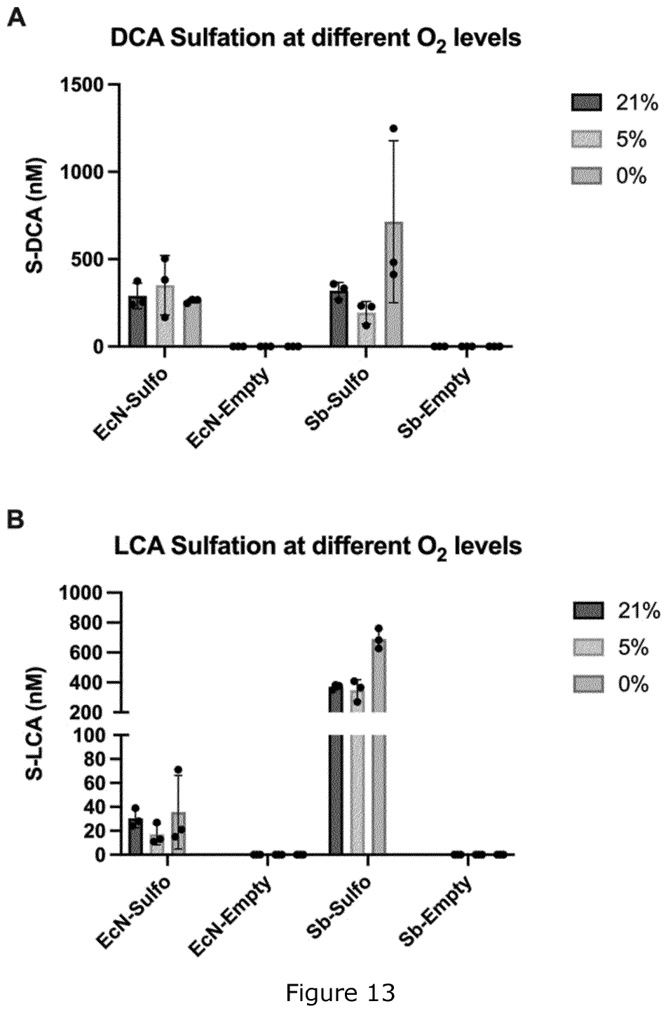

E. coli Nissle 1917 and S. boulardii expressing SULT2A1 was found to sulfate secondary bile acids (DCA and LCA) under different oxygen concentrations (FIG. 13), resembling conditions encountered in vivo, for example in the GI tract.

### Example 7—Sulfation in Faecal Matrices

Methods

Precultures of the strains were made in 2xYT supplemented with kanamycin, cultures were incubated overnight in a shaking incubator at 37° C. The following day preculture was used to inoculate fecal suspension matrixes (FM) at a ratio of 1:25. FM were prepared using frozen fecal samples diluted 1 g in 10 mL of phosphate-buffered saline (PBS). Tubes were vortexed until and homogenous suspension was achieved and were subsequently centrifuged at at 100 g for 10 minutes, following another centrifugation step at 150 g for 5 minutes. Supernatant was decanted and frozen into working stocks of 10 mL. On the day of the experiment, FM stock was thawed at room temperature and the different FM conditions were prepared adding MgSO4 for a final concentration of 2 mM, kanamycin for a final concentration of 50 μg/ml and DCA/LCA for a final concentration of 100 M. Once inoculated, 96 deep well plates were incubated at 37° C. in a shaking incubator for aerobic growth or in an anaerobic container with anaerobic atmosphere generation bags placed in a fixed 37° C. incubator. After 24 hours of incubation, cultures were centrifuged at 5000 g for 10 minutes, supernatant was taken and stored at −20° C. until processing for LC-MS/MS. Analytics were performed as previously described. Student T-test was used to compare the strains tested.

Results

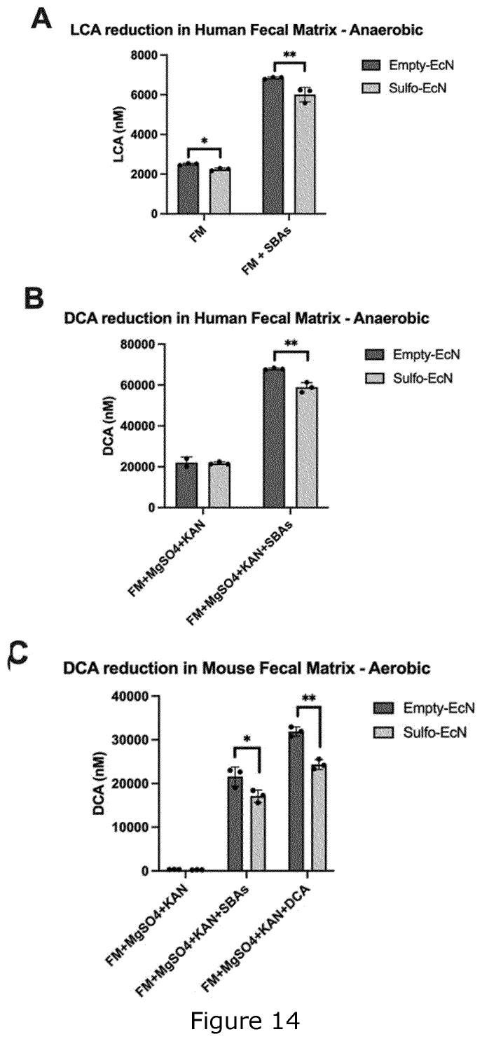

The results presented in FIG. 14 shows that E. coli Nissle 1917 expressing SULT2A1 can decrease the levels of secondary bile acids, DCA and LCA, in fecal suspensions from human and mouse. This validates the capacity of the strain to be active in vivo and maintaining the potential to lower specific secondary bile acids.

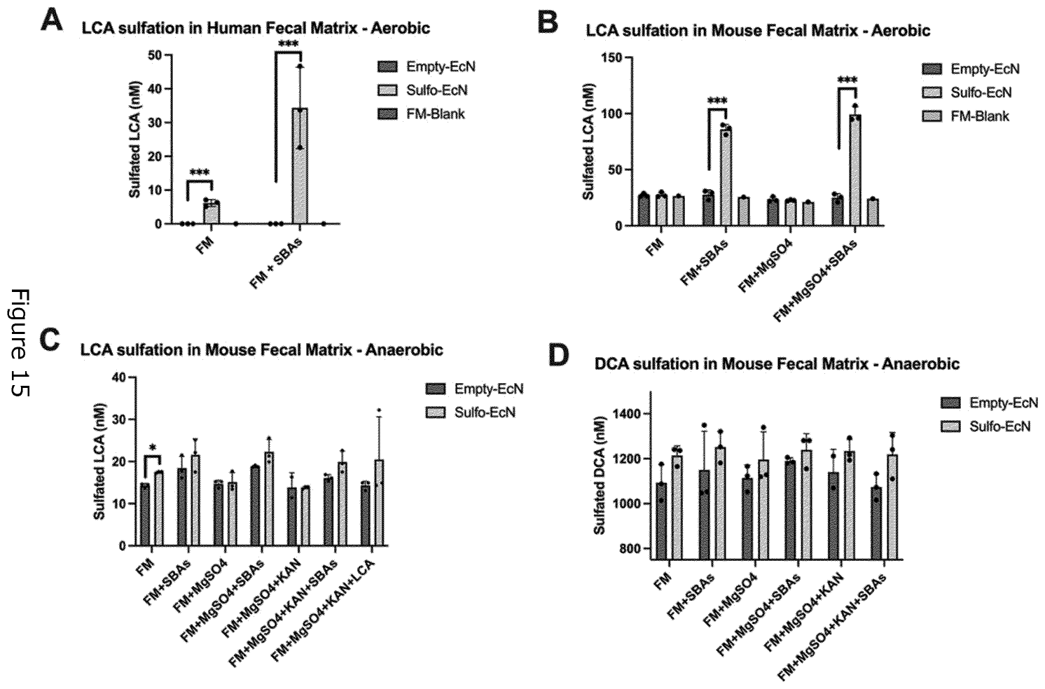

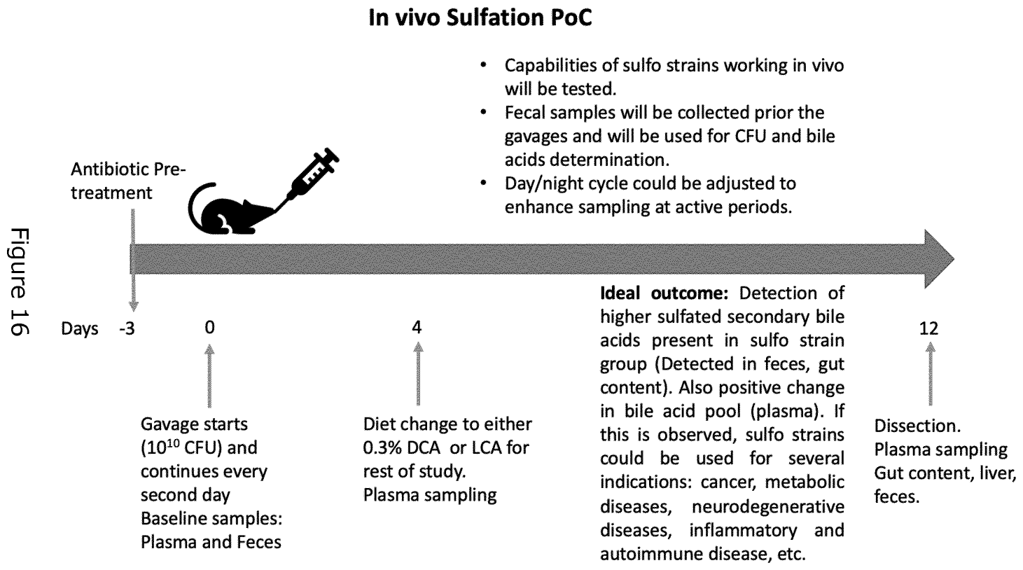

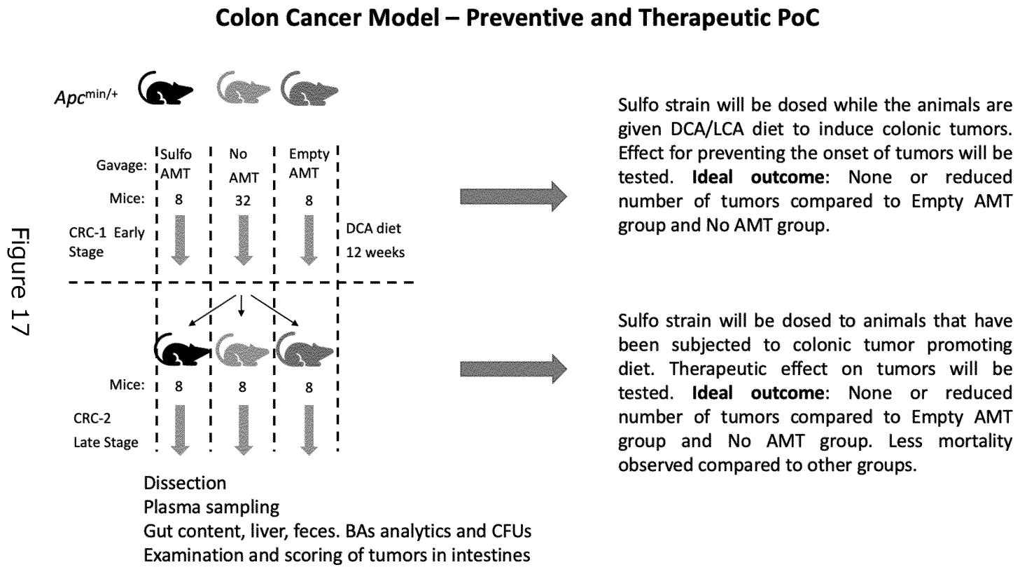

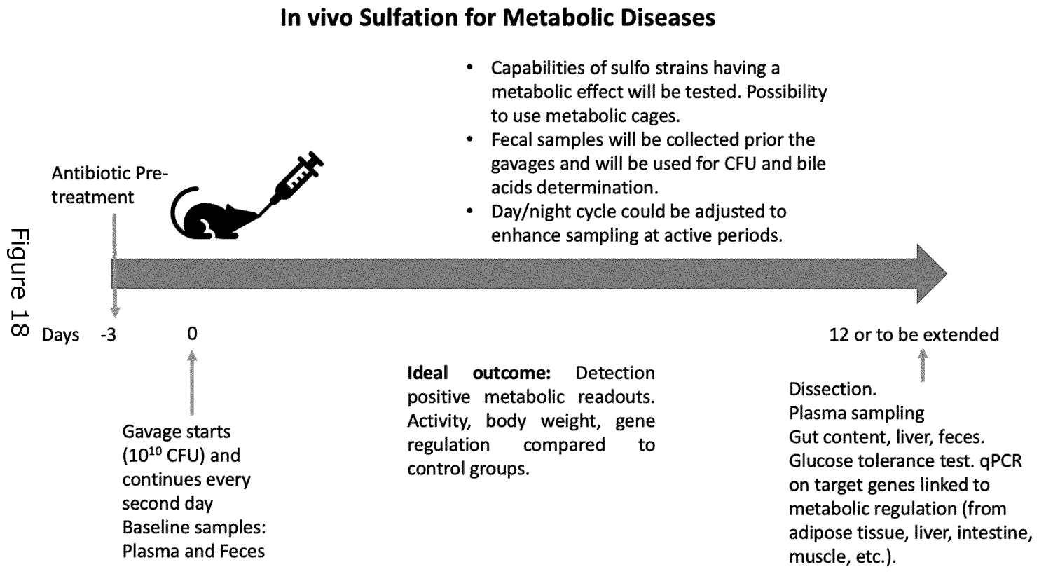

The results presented in FIG. 15 shows sulfation activity of E. coli Nissle 1917 expressing SULT2A1 in a biologically relevant matrix for both LCA and DCA. It is worth noticing that these matrixes have a complex composition, however, the SULT2A1 expressed by E. coli Nissle 1917 holds affinity towards DCA and LCA. The low levels of sulfation anaerobically could be a result of the pre-culture growing aerobically and anaerobically. Usually, the cells must adapt to anaerobic metabolism before being exposed to the experimental condition, this could explain the low performance here. Even though low levels of sulfation anaerobically, a trend is clear where more sulfated secondary bile acids are observed in samples from the sulfating strain.

### SPECIFIC EMBODIMENTS

1. A microbiome-based therapeutic composition comprising an engineered probiotic cell expressing a sulfotransferase.
2. The microbiome-based therapeutic composition according to item 1, wherein the probiotic cell is a bacterium or yeast.
3. The microbiome-based therapeutic composition according to item 1 or 2, wherein the probiotic cell is a bacterium selected from the group consisting of Lactobacillus spp., Bifidobacterium Spp., and Escherichia spp.
4. The microbiome-based therapeutic composition according to any of the preceding items, wherein the probiotic cell is a bacterium is selected from Escherichia species.
5. The microbiome-based therapeutic composition according to any of the preceding items, wherein the probiotic cell is Escherichia coli.
6. The microbiome-based therapeutic composition according to any of the preceding items wherein the probiotic cell is E. coli Nissle 1917.
7. The microbiome-based therapeutic composition according to item 1 or 2, wherein the probiotic cell is selected form Saccharomyces species.
8. The microbiome-based therapeutic composition according to item 7, wherein the probiotic cell is Saccharomyces boulardii.
9. The microbiome-based therapeutic composition according to any of the preceding items, wherein the engineered probiotic cell expresses a human sulfotransferase.
10. The microbiome-based therapeutic composition according to any of the preceding items, wherein the sulfotransferase is human SULT2A1, as shown in SEQ ID NO: 29, or a functional homologue thereof, having at least 80% sequence identity, such as 90%, such as 95% such as 99% sequence identity to SEQ ID NO: 29.
11. The microbiome-based therapeutic composition according to any of the preceding items, wherein SULT2A1 is codon optimized for expression in the probiotic cell.
12. The microbiome-based therapeutic composition according to any of the preceding items, wherein SULT2A1 is codon optimized for expression in E. coli, as shown in SEQ ID NO: 9.
13. The microbiome-based therapeutic composition according to any of the preceding items, wherein SULT2A1 is codon optimized for expression in Saccharomyces boulardii, as shown in SEQ ID NO: 64.
14. The microbiome-based therapeutic composition according to any of the preceding items, wherein the expression of SULT2A1 is obtained by transformation of said probiotic cell with a plasmid.
15. The microbiome-based therapeutic composition according to any of the preceding items, wherein the expression of SULT2A1 is obtained by transformation of said probiotic cell with a pMUT plasmid.
16. The microbiome-based therapeutic composition according to any of the preceding items, wherein the expression of SULT2A1 is obtained by transformation of said probiotic cell with a pCF plasmid.
17. The microbiome-based therapeutic composition according to any of items 1-13, wherein the expression of a sulfotransferase is obtained by genomic integration.
18. The microbiome-based therapeutic composition according to any of items 1-13, wherein the plasmid according to any of items 14-16, or the integrated gene according to item 17, comprises an inducible or constitutive promoter.
19. The microbiome-based therapeutic composition according to any of items 1-13, wherein the plasmid according to any of items 14-16, or the integrated gene according to item 17, comprises an inducible promoter, as shown in SEQ ID NO: 60 or a functional variant thereof, wherein the nucleic acid sequence of the inducible promoter has at least 80% sequence identity to SEQ ID NO: 60.
20. The microbiome-based therapeutic composition according to any of items 1-13, wherein the plasmid according to any of items 14-16, or the integrated gene according to item 17, comprises a constitutive promoter, as shown in SEQ ID NO: 61 or SEQ ID NO: 62 or a functional variant thereof, wherein the nucleic acid sequence of the inducible or constitutive promoter has at least 80% sequence identity to SEQ ID NO: 61 or SEQ ID NO: 62.
21. The microbiome-based therapeutic composition according to claim 20, wherein the plasmid according to any of items 14-16, or the integrated gene according to item 17, further expresses one or more genes resulting in an increased sulfate uptake.
22. The microbiome-based therapeutic composition according any of the preceding items, comprises a Bacillus subtilis cysP gene, as shown in SEQ ID NO: 28, wherein the nucleic acid sequence of the cysP gene has at least 70%, such as at least 80%, such as at least 90%, such as at least 95% or such as at least 99% sequence identity to SEQ ID NO: 28.
23. The microbiome-based therapeutic composition according to any of the preceding items, wherein the probiotic cell is further genetically engineered, so that the expression of one or more genes, selected from the group consisting of cysZ, sbp, cysP, cysU, cysW, cysA, cysD, cysN, cysC and cysQ, encoding one or more sulfate permeases, sulfate permease related genes or sulfate recycling related genes, is/are upregulated.
24. The microbiome-based therapeutic composition according to any of the preceding claims, wherein the probiotic cell is further genetically engineered, so that the expression of one or more genes, selected from the group consisting of cysP, cysU, cysW, cysA, cysD, cysN, cysC and cysQ, encoding one or more sulfate permeases, sulfate permease related proteins or sulfate recycling related proteins of SEQ ID NOs: 44-51, is/are regulated by a promoter, wherein the protein of any one of SEQ ID NO: 44-51, or a functional homologue thereof, has at least 80% sequence similarity to any one of SEQ ID NOs: 44-51 and wherein a functional homologue of any one of SEQ ID NOs: 44-51, has at least 50% functionality of said protein.
25. The microbiome-based therapeutic composition according to any of the preceding items, wherein the probiotic cell is further genetically engineered, so that the expression of one or more genes, selected from the group consisting of cysP, cysU, cysW and cysA, encoding one or more sulfate permeases, sulfate permease related proteins or sulfate recycling related proteins of SEQ ID NOs: 44-47, is/are upregulated, wherein the protein of any one of SEQ ID NOs: 44-47, or a functional homologue thereof, has at least 80% sequence similarity to any one of SEQ ID NOs: 44-47 and wherein a functional homologue of any one of SEQ ID NOs: 44-47, has at least 50% functionality of said protein.
26. The microbiome-based therapeutic composition according to any of the preceding items, wherein the probiotic cell is further genetically engineered, so that the expression of one or more genes, selected from the group consisting of cysD, cysN, cysC and cysQ encoding one or more sulfate permeases, sulfate permease related proteins or sulfate recycling related proteins of SEQ ID NOs: 48-51, is/are upregulated, wherein the protein of any one of SEQ ID NOs: 48-51, or a functional homologue thereof, has at least 80% sequence similarity to any one of SEQ ID NOs: 48-51 and wherein a functional homologue of any one of SEQ ID NOs: 48-51, has at least 50% functionality of said protein.
27. The microbiome-based therapeutic composition according to any of the preceding items, wherein the probiotic cell is further genetically engineered, so that the expression of one or more genes, selected from the group consisting of cysD, cysN, and cysC encoding one or more sulfate permeases, sulfate permease related proteins or sulfate recycling related proteins of SEQ ID NOs: 48-51, is/are upregulated, wherein the protein of any one of SEQ ID NOs: 48-51, or a functional homologue thereof, has at least 80% sequence similarity to any one of SEQ ID NOs: 48-51 and wherein a functional homologue of any one of SEQ ID NOs: 48-51, has at least 50% functionality of said protein.
28. The microbiome-based therapeutic composition according to any of the preceding items, wherein the probiotic cell is further genetically engineered so that one or more sulfatase genes of said probiotic cell is/are at least partially inactivated.
29. The microbiome-based therapeutic composition according to any of the preceding items, wherein the probiotic cell is further genetically engineered so that one or more of the sulfatase(s) and sulfatase related gene(s), selected from the group consisting of yjcS, as/A, ydeN, yidJ, ydeM aslB, hdhA, cysQ, cysH and acrB with a nucleic acid sequence according to SEQ ID NOs: 53-59 and SEQ ID NOs: 69-70 respectively, is/are at least partially inactivated.
30. The microbiome-based therapeutic composition according to any of the preceding items, wherein the probiotic cell is further genetically engineered so that one or more gene(s) selected from the group consisting of ydeN, cysQ, cysH and acrB, with a nucleic acid sequence according to SEQ ID NOs: 55, 69, 70 and 71 respectively is/are at least partially inactivated.
31. The microbiome-based therapeutic composition according to any of the preceding items for use as a medicament.
32. The microbiome-based therapeutic composition for use according to item 31, wherein the medicament is for use in the treatment of cancer and/or inflammatory diseases.
33. The microbiome-based therapeutic composition for use according to any of items 31-32, wherein the medicament is for use in the treatment of colon cancer.
34. The microbiome-based therapeutic composition for use according to any of items 31-32, wherein the medicament is for use in ameliorating cancer and/or inflammatory disease(s).
35. The microbiome-based therapeutic composition for use according to item 31, wherein the medicament is for use in ameliorating colon cancer.
36. The microbiome-based therapeutic composition for use according to item 31, wherein the medicament is for use in inhibiting cancer and/or inflammatory disease(s).
37. The microbiome-based therapeutic composition for use according to item 31, wherein the medicament is for use in inhibiting colon cancer.
38. The microbiome-based therapeutic composition for use according to item 31, wherein the medicament is for use in the treatment of a metabolic disorder.
39. The microbiome-based therapeutic composition for use according to any of items 31-38 wherein the composition is designed for administered by oral or rectal administration.
40. The microbiome-based therapeutic composition for use according to any of items 31-39, wherein the composition is provided as a tablet, capsule or suppository.
41. The microbiome-based therapeutic composition for use according to any of items 31-40, wherein the composition is provided as prophylactic treatment.
42. The microbiome-based therapeutic composition for use according to any of items 31-41, wherein the composition is administered once or repeatedly.
43. A microbiome-based therapeutic composition according to any of items 1-30 for use in bacterial sulfation of secondary bile acids for treating and/or preventing bile acid disorders and/or complications resulting from and/or leading to unbalanced bile acid pools.
44. The microbiome-based therapeutic composition for use according to item 43, wherein said secondary bile acids are selected from the group consisting of LCA, DCA, TDCA, GDCA, GLCA, UDCA, TLCA, TUDCA and GUDCA.
45. A microbiome-based therapeutic regimen for modulating the concentrations of bile acids via sulfation, wherein a microbiome-based therapeutic composition according to any of items 1-30 is administered by oral or rectal administration.
46. A microbiome-based therapeutic regimen for modulating the concentrations of bile acids via sulfation, wherein a microbiome-based therapeutic composition according to any of items 1-30 wherein the composition is administered by fecal microbiota transplantation.
47. The microbiome-based therapeutic regimen according to any one of items 45-46, which is administered once or repeatedly.
48. The microbiome-based therapeutic regimen according to any one of items 45-47, which is provided as a tablet, capsule or suppository.

1. A microbiome-based therapeutic composition comprising:
an engineered probiotic cell expressing a sulfotransferase, wherein the sulfotransferase is human SULT2A1, as shown in SEQ ID NO: 29, or a functional homologue thereof, having at least 80% sequence identity to SEQ ID NO: 29.

2-27. (canceled)

28. The microbiome-based therapeutic composition according to claim 1, wherein the probiotic cell is E. coli Nissle 1917 or Saccharomyces boulardii.

29. The microbiome-based therapeutic composition according claim 1, wherein SULT2A1 is codon optimized for expression in the probiotic cell.

30. The microbiome-based therapeutic composition according to claim 1, wherein the SULT2A1 is codon optimized for expression in E. coli, as shown in SEQ ID NO: 9 or for expression in Saccharomyces boulardii as shown in SEQ ID NO: 64.

31. The microbiome-based therapeutic composition according to claim 1, wherein the expression of SULT2A1 is obtained by transformation of said probiotic cell with a pMUT plasmid or pCF plasmid.

32. The microbiome-based therapeutic composition according to claim 1, wherein the expression of a sulfotransferase is obtained by genomic integration of a nucleic acid sequence encoding said sulfotransferase.

33. The microbiome-based therapeutic composition according claim 31, wherein the plasmid comprises an inducible promoter, as shown in SEQ ID NO: 60 or a constitutive promoter, as shown in SEQ ID NO: 61 or SEQ ID NO: 62.

34. The microbiome-based therapeutic composition according to claim 1 further comprising a Bacillus subtilis cysP gene, as shown in SEQ ID NO: 28, wherein the nucleic acid sequence of the cysP gene has at least 70% sequence identity to SEQ ID NO: 28.

35. The microbiome-based therapeutic composition according to claim 1 wherein the probiotic cell is further genetically engineered, so that the expression of one or more genes, selected from the group consisting of cysP, cysU, cysW, cysA, cysD, cysN, cysC, and homologues thereof, encoding one or more sulfate permeases, sulfate permease related proteins or sulfate recycling related proteins according to SEQ ID NOs: 44-51, is/are regulated by a promoter and wherein the cysP, cysU, cysW, cysA, cysD, cysN, cysC according to any one of SEQ ID NOs:
44-51, or a functional homologue thereof, has at least 80% sequence similarity to any one of SEQ ID NOs: 44-51 and wherein a functional homologue of any one of SEQ ID NOs: 44-51, has at least 50% functionality of said protein.

36. The microbiome-based therapeutic composition according to claim 1, wherein the probiotic cell is further genetically engineered so that one or more sulfatase genes of said probiotic cell is/are at least partially inactivated.

37. The microbiome-based therapeutic composition according to claim 1, wherein the probiotic cell is further genetically engineered so that one or more of the sulfatase and sulfatase related gene(s), selected from the group consisting of yjcS, aslA, ydeN, yidJ, ydeM aslB, hdhA, cysQ, cysH and acrB with a nucleic acid sequence according to SEQ ID NOs: 53-59 and SEQ ID NOs: 69-70 respectively, is/are at least partially inactivated.

38. The microbiome-based therapeutic composition according to claim 1, wherein the probiotic cell is further genetically engineered so that one or more of the sulfatase and sulfatase related gene(s), selected from the group consisting of ydeN, cysQ, cysH and acrB, with a nucleic acid sequence according to SEQ ID NOs: 55, 69, 70 and 71 respectively is/are at least partially inactivated.

39. A method of treating or inhibiting a cancer or an inflammatory disease in a subject comprising:
administering the microbiome-based therapeutic composition of claim 1 to a subject in need thereof.

40. The method of claim 39, wherein said cancer is a colon cancer.

41. A method of treating or inhibiting a metabolic disorder in a subject comprising:
administering the microbiome-based therapeutic composition of claim 1 to a subject in need thereof.

42. The method of claim 39, wherein the composition is administered by oral or rectal routes.

43. The method of claim 39, wherein the composition is administered by fecal microbiota transplantation.

44. The method of claim 39, wherein the composition is administered as a tablet, capsule or suppository.

45. The method of claim 39, wherein the composition is administered prophylactically.

46. The method of claim 39, wherein the composition is administered once or repeatedly.

47. A method of treating or inhibiting a bile acid disorder and/or a complication resulting from and/or leading to unbalanced bile acid pools in a subject comprising:
administering the microbiome-based therapeutic composition of claim 1 to a subject in need thereof.

48. The method according to claim 47, wherein said secondary bile acids are selected from the group consisting of LCA, DCA, TDCA, GDCA, GLCA, UDCA, TLCA, TUDCA and GUDCA.


SEQUENCE LISTING (74 sequences)

SEQ ID NO: 1 - DNA, 876 bp, Rattus norvegicus
  SULT1A1, pst-5 sequence, GeneBank AF394783.1
atggagttctcccgtccaccgctagtgcatgtgaagggtatcccactcatcaaatactttgcagagacaattgggccattgcagaacttcacagcctggcctgatgacttgctgatcagcacatacccaaagtctggtactacctggatgagtgagatcctggatatgatctatcagggtggcaagctagagaagtgtggccgcgcccccatctatgcccgggtacccttccttgagttcaaatgtccaggggttccctcaggtcttgaaactttggaagagacaccagccccacggctccttaagacacatctgcccctgtccttgctccctcagagtctgctggatcagaaggtcaaggtgatctacattgcccgaaatgcaaaggatgtggttgtctcctattataacttctacaacatggccaagctgcaccctgatccaggcacctgggacagcttcttggagaacttcatggatggggaagtgtcctatgggtcgtggtaccagcacgtgaaggagtggtgggagctgagacacactcaccctgttctctatctcttctatgaagacataaaggagaaccccaaaagggagatcaagaagattctagagtttttggggcgctctctacccgaggagactgtggattccattgttcaccacacatctttcaagaaaatgaaagagaactgcatgactaactacacaaccatccccactgagattatggaccacaatgtttctcccttcatgaggaaaggtactactggggactggaaaaataccttcactgtagcccagaatgagcgctttgatgcccactatgctaagacaatgacagattgtgacttcaagtttcgttgtgaactatga

SEQ ID NO: 2 - DNA, 918 bp, Rattus norvegicus
  SULT1A1-clone 2, pst-26
atggagttctcccgtccaccgctagtgcatgtgaagggtatcccactcatcaaatactttgcagagacaattgggccattgcagaacttcacagcctggcctgatgacttgctgatcagcacatacccaaagtctggtactacctggatgagtgagatcctggatatgatctatcagggtggcaagctagagaagtgtggccgcgcccccatctatgcccgggtacccttccttgagttcaaatgtccaggggttccctcaggtcttgaaactttggaagagacaccagccccacggctccttaagacacatctgcccctgtccttgctccctcagagtctgctggatcagaaggtcaaggtgatctacattgcccgaaatgcaaaggatgtggttgtctcctattataacttctacaacatggccaagctgcaccctgatccaggcacctgggacagcttcttggagaacttcatggatggggaagtgtcctatgggtcgtggtaccagcacgtgaaggagtggtgggagctgagacacactcaccctgttctctatctcttctatgaagacataaaggagaaccccaaaagggagatcaagaagattctagagtttttggggcgctctctacccgaggagactgtggattccattgttcaccacacatctttcaagaaaatgaaagagaactgcatgactaactacacaaccatccccactgagattatggaccacaatgtttctcccttcatgaggaaaggtactactggggactggaaaaataccttcactgtagcccagaatgagcgctttgatgcccactatgctaagacaatgacagattgtgacttcaagtttcgttgtgaactacgacaagcttgcggccgcataatgcttaagtcgaacagaaagtaa

SEQ ID NO: 3 - DNA, 873 bp, Rattus norvegicus
  codon optimized SULT1A1, pst-25
atggaattttcacgtccgcctctggttcatgttaaaggtattccgctgattaaatacttcgccgaaaccattggtccgctgcagaattttaccgcatggcctgatgatctgctgattagcacctatccgaaaagcggcaccacctggatgagcgaaattctggatatgatttatcagggtggcaaactggaaaaatgtggtcgtgcaccgatttatgcacgtgttccgtttctggaattcaaatgtccgggtgttccgagcggtctggaaaccctggaagaaacaccggcaccgcgtctgctgaaaacccatctgccgctgagcctgctgccgcagtcactgctggatcagaaagttaaagttatctatattgcccgtaacgccaaagatgttgtggtgagctattataacttctataacatggcaaaactgcatccggatcctggcacctgggatagctttctggaaaactttatggatggtgaagttagctatggtagctggtatcagcatgtgaaagaatggtgggaactgcgtcatacccatccggttctgtacctgttttatgaagatatcaaagagaacccgaaacgcgaaatcaaaaaaatcctggaatttctgggtcgtagcctgccggaagaaaccgttgatagcattgttcatcataccagcttcaaaaaaatgaaagaaaactgcatgaccaactacaccaccattccgaccgaaattatggatcacaatgttagcccgtttatgcgtaaaggcaccaccggtgattggaaaaatacatttaccgttgcacagaacgaacgctttgatgcacattatgcaaaaaccatgaccgactgcgatttcaaatttcgttgtgaactg

SEQ ID NO: 4 - DNA, 888 bp, Canis lupus familiaris
  SULT1A1, pst-13
atggaggacattcccgacacctcgcgcccaccactgaagtatgtgaaggggatccctctaatcaagtactttgcagaggcactggagtcactgcaggacttccaagctcagcctgatgacctgcttatcagcacgtaccccaaatctggcaccacttgggtgagcgagatcctggacatgatctaccaagacggtgatgtagagaagtgtcgcagggcccccgtcttcatccgggtacccttccttgagttcaaggctccagggattcccacaggtttggaggttctaaaagatacaccagccccacgactcatcaagacacacctgcccctggccctgctcccccagaccttgttggatcagaaggtcaaggtggtctacgtcgcccgcaacgcaaaagatgtagctgtctcctattaccacttctaccgcatggccaaggtgcaccctgaccctgacacctgggacagcttcctggagaagttcatggctggggaagtgtcctatgggtcctggtatcagcatgtgcaggaatggtgggagctgagtcacactcaccctgttctctacctcttctatgaggacatgaaagagaaccccaaaagggagattcagaagatcctgaagtttgtggggcgctccctgccagaggagactgtggatctcattgtccagcacacgtctttcaaggagatgaagaacaactccatggctaactacaccaccttatctcctgacatcatggaccacagcatttctgccttcatgaggaaaggcatctcgggggactggaagaccaccttcactgtggcccagaatgagcgctttgatgccgactatgccaagaagatggagggttgtggcctcagcttccgcacacagctgtga

SEQ ID NO: 5 - DNA, 888 bp, Sus scrofa domesticus
  SULT1A1, pst-14
atggagccggtccaggacacctaccgcccgccactggagtacgtgaagggggtccctctcatcaagtactttgcagaggcactggggccactggagagtttccaagcttggcccgatgacgtgctgatcagcacctatcccaaatctggtaccacctgggtgagcgagatcctggacctaatctaccagggtggtgacctgcagaagtgtcagagagcccccatctttgtgcgggtgcccttccttgagttcaaaatccccgggtgtcccacaggttttgagctgctgaaagatacaccagccccacggctcctcaagacacacttgcccctggccctgctaccccagactctgctggaccagaaggtcaaggtggtctacgtcgcccgcaacgcaaaggacgtggccgtctcctattaccacttctaccgtatggccaaagtgcaccctaaccctggcacgtgggacagcttcctggaagacttcatggccggagaagtgtcctacgggtcctggtaccagcacgtgcaggagtggtgggagctgcgtcacacccaccctgtcctctatctcttctacgaggacatgaaggagaaccccaagagggagattcagaagatcctggagtttgtggggcgctcgctgccagaggagacggtggaggacatcgtccagcacacgtccttccaggagatgaagaacaacgccatgaccaactaccgcaccctcccctccgacctcctggaccacagcatctcggccttcatgaggaaaggcatcactggagattggaaaagcaccttcacagtggcccagaacgagcgctttgaggccgactatgctgagaagatggcaggctgtaacctccgcttccgctctgagctgtga

SEQ ID NO: 6 - DNA, 888 bp, Equus caballus ferus
  SULT1A1, pst-11
atggagctgatccaggacacctcccgcccaccactgaagtatgtgaaaggggtccctctcatcaagtactttgcagaggcactggggccactgcagagcttccaagcccggcctgatgacctgctcatcagcacctaccccaaatctggcaccacgtgggtgagcgagatcctggacatgatctaccatggtggtgaccttgaaaagtgtcgcagggcccctatcttcatccgagtgcccttccttgagtttaaggccccagagattccctcaggtgtcgaggttctgaaagatacaccagccccacggctcctcaagacacacttgcccctgtccctgctcccccaaaccttgctggatcagaaggtcaaggtggtgtaccttgcccgcaatgcaaaggatgtggccgtctcctattaccacttctaccgcatggccaaagtgcaccctgaccctggcacctgggacagcttcctggagaagttcatggctggggaagtgtcctacgggtcttggtacaagcacgtgcaggagtggtgggagctgagtcacacccaccccgtcctctatctcttctatgaggacatgaaagagaaccccaaaaaggagattcagaagatcctggagtttgtggggcgttccctgccagaggagaccttagatcgcattgtccagcacacatctttcaaggagatgaagaagaaccccatggctaactacagcaccatcccctgtgacatcatggaccacaacatttctgccttcatgaggaaaggcatcgcgggggactggaaaaacaccttcaccgtggcccagaacgagcactttgacaccgactatgctgagaagatggcaggctgcaaactcagcttccgctcagaggtgtga

SEQ ID NO: 7 - DNA, 888 bp, Homo sapiens
  SULT1A1, pst-4
atggaactgattcaggataccagccgtccgcctctggaatatgttaaaggtgttccgctgattaaatactttgcagaagcactgggtccgctgcaaagctttcaggcacgtccggatgatctgctgattagcacctatccgaaaagcggcaccacctgggttagccagattctggatatgatttatcagggtggtgatctggaaaaatgtcatcgtgcaccgatttttatgcgtgttccgtttctggaatttaaagcaccgggtattccgagcggtatggaaaccctgaaagatacaccggcaccgcgtctgctgaaaacccatctgccgctggcactgctgccgcagaccctgctggatcagaaagttaaagttgtttatgttgcccgtaacgccaaagatgttgcagttagctattatcacttttatcacatggccaaagttcatccggaaccgggtacatgggatagctttctggaaaaattcatggttggtgaagttagctacggtagctggtatcagcatgttcaagaatggtgggaactgagccgtacccatccggttctgtacctgttttatgaagatatgaaagagaacccgaaacgcgagattcagaaaatcctggaatttgttggtcgtagcctgccggaagaaaccgttgattttatggtgcagcataccagcttcaaagaaatgaaaaaaaacccgatgaccaactataccaccgttccgcaagaatttatggatcattcaattagcccgtttatgcgtaaaggtatggcaggcgattggaaaaccacctttaccgttgcacagaatgaacgttttgatgcagattatgccgaaaaaatggcaggttgtagcctgagctttcgtagcgaactgtaa

SEQ ID NO: 8 - DNA, 858 bp, Homo sapiens
  SULT2A1, pst-50
atgtcggacgatttcttatggtttgaaggcatagctttccctactatgggtttcagatccgaaaccttaagaaaagtacgtgatgagttcgtgataagggatgaagatgtaataatattgacttaccccaaatcaggaacaaactggttggctgagattctctgcctgatgcactccaagggggatgccaagtggatccaatctgtgcccatctgggagcgatcaccctgggtagagagtgagattgggtatacagcactcagtgaaacggagagtccacgtttattctcctcccacctccccatccagttattccccaagtctttcttcagttccaaggccaaggtgatttatctcatgagaaatcccagagatgttttggtgtctggttattttttctggaaaaacatgaagtttattaagaaaccaaagtcatgggaagaatattttgaatggttttgtcaaggaactgtgctatatgggtcatggtttgaccacattcatggctggatgcccatgagagaggagaaaaacttcctgttactgagttatgaggagctgaaacaggacacaggaagaaccatagagaagatctgtcaattcctgggaaagacgttagaacccgaagaactgaacttaattctcaagaacagctcctttcagagcatgaaagaaaacaagatgtccaattattccctcctgagtgttgattatgtagtggacaaagcacaacttctgagaaaaggtgtatctggggactggaaaaatcacttcacagtggcccaagctgaagactttgataaattgttccaagagaagatggcagatcttcctcgagagctgttcccatgggaataa

SEQ ID NO: 9 - DNA, 858 bp, Homo sapiens
  codon optimized SULT2A1, pst-51
atgtcggacgattttctgtggttcgaaggaattgcgtttcctaccatggggttccgttcggagaccttgcgcaaggtgcgcgatgaatttgtgatccgtgacgaagacgtaattatcctgacttatcccaaatcaggcaccaattggttagcagagattttatgtttgatgcattccaaaggagatgccaaatggatccagtcggtgccgatttgggaacgtagtccttgggttgagtcagaaattggttatacggcattaagtgaaactgaaagtcctcgcttgttttcctctcacttgccgatccaactttttccgaaaagtttcttttcgagcaaagcaaaggtgatctatcttatgcgcaacccgcgtgacgtattagtaagcggttatttcttctggaaaaacatgaagttcattaaaaagcccaagtcatgggaagagtattttgaatggttctgccagggaactgtgctttatgggtcatggtttgaccacatccacggctggatgcctatgcgtgaagagaagaactttttgttattatcatacgaggagttgaagcaggacacaggacgtactatcgagaaaatttgccaattcttaggaaagacccttgagcctgaggaactgaacctgattcttaaaaactcctcattccagtccatgaaggagaataagatgtccaattacagcctgttgtctgtagactacgtcgtggataaagctcaattattacgcaaaggggtgtctggtgactggaagaatcacttcaccgtggcgcaggccgaagactttgataaattgttccaggaaaaaatggctgacctgccgcgcgagttattcccgtgggagtaa

SEQ ID NO: 10 - DNA, 897 bp, Danio rerio
  SULT1ST1, pst-29, GeneBank LP858384.1
atggacatcccggattttagcagcattagcagccgtccgaccatttttgaatttgaaggtattagcatgatcaaccactttaccgaaaactgggagaaagtgaaaaactttcaggcacgtccggatgatattctgattgcaacctatccgaaagcaggcaccacctgggttagctatattctggatctgctgtattttggtgaaaacgcaccggaagaacataccagccagccgatttatatgcgtgttccgtttctggaaagctgctttaaagttattgcaagcggcaccgaactggcagataatatgaccaccagtccgcgtctgattaaaacccatctgccggttcagctgattccgaaaagtttttgggaacagaatagccgtgttgtttatgttgcacgtaatgccaaagataacgtggtgagctattttcattttgatcgcatgaatatcgttgaaccggatcctggtgattggaatacctttctgcatcgttttatggatggcaaaagcgtttttggtccgtggtatgatcacgttaatggctattgggaaaaaaaacagacctatagcaacctgctgtacctgttttatgaagatctggttgaagataccggtcgtgaagttgatcgtctgtgtagctttctgggtctgagcaccagcgttagcgatcgtgaaaaaatcaccaaagatgttcagttcgatgccatgaaacagaacaaaatgaccaattatagcaccctgccggtgatggatttcaaaatttcaccgtttatgcgcaaaggcaaagttggcgattggaaaaatcattttaccgtggcacagaacgaacagtttgatgaggtgtacaaagaaaaaatgaaaaacgccaccgtgaaatttcgcaccgaaatc

SEQ ID NO: 11 - DNA, 888 bp, Danio rerio
  SULT6B1, pst-31, GeneBank LP858386.1
atgagccagatgaaaagccgtatggaaaccgcagcaaaaatgaaagatgaggataaactgtatcgccgtgatggtattctgtatagcaccgttctgagccctccggaaaccctggacaaactgaaagatctgcaggcacgtgaagatgatctgattctggttgcatatccgaaatgcggttttaattggatggttgcagttctgcgcaaaatcattaatgcaagcaccggcaaagatgaaaaaccgcctgaacgtccgcctctggttgaatttctgcctccgaccgttcaagaagaaatggcccagatgcctccgcctcgtctgctgggcacccatctgcatccggataatatgcctgcaaccttttttaccaaaaaaccgaaaatcctggtggtgtttcgtaatccgaaagataccctggttagctattatcactttatgaacaaaaatccggttctgccgaatgcagaaagctgggataaattctttagcgattttatgaccggtgatgttagctggggtagctattttgatcatgcactggcatgggaaaaacgtattgatgatccgaatgtgatgatcgtgatgtatgaagatctgaaacagaatctgccggaaggcgtgaaaaaaatcagcgaatttttcagcctgccgctgaccgatgaacaggttagcagcattgcaggtcagagcacctttagcgcaatggttgaaaattcacagaaaagccatggtaactttggcagcatcttttttcgtaaaggtgaagtgggtgattggaaaaaccattttagcgaagcacagagcaaacaaatggatgagctgtatcatagcaaactggcaggtacaaaactggctgcacgtatgaattatgatctgtattgccag

SEQ ID NO: 12 - DNA, 1017 bp, Drosophila melanogaster
  dmST1, pst-6
atgccccagtcgagcttctttgccaagagcgtgcccttcgagcagatcgacaagctggccatcagtggcggctactcctcgatcttcgccagcagcaagccgtcggtgccggtggtcgggaactgggagcagcgtttctgccgcctagcggacactttccagccggtcctggatcgggtgtacgacttcgaggtgcgggacgacgatgtgtggattgtcacgctgccaaaatgcggcaccacctggatgcaggagctggcctggctggtgatcaatgagtgtgattttgagacggccaagagcgtggatctcacgcatcgatcgcccttccttgaattcaatggcgtggttcccaatgtaccacacgacaccatagccgcagcgaatgccctgccatcgccgcgcctgatcaagtcccatctgcccgcctggatgctgccgaggcagatctggagcaagaggcccaagatcatttatgtgtaccgcaacccgaaggacgcggccatctcgtacttccaccactggcgtggaatggtgggctaccagggcaccaagtcggacttcatgcactcctttatcgacggctatgtgaacttcacgccctgctggccgcacatcctggacttttggcagctgcgccacgagcccaacatcttcttcaccagctacgagcggatgaagggccagctgggccaggttatctcggaggtggcccagttcctggagcgcagtgtcagtggggagcagatccagcagatgcagcgacacctctccttcgagagcatgcgcgacaatcccgcctgcaaccacgtcaaggagttcgagagcatgaaggcggcagccggcagggaggtggaggagttcaggttcgttcgccgcggagtcgtgggcagccacaaggatgagctcaccgccgacatcatccgggaattcgatctctggtccgatagtaatctgcgggatttcaaattgaacatggatgactttgccaactacagcaagtttgcgtccacgtaa

SEQ ID NO: 13 - DNA, 984 bp, Drosophila melanogaster
  dmST1 - clone2, pst-27
atgccccagtcgagcttctttgccaagagcgtgcccttcgagcagatcgacaagctggccatcagtggcggctactcctcgatcttcgccagcagcaagccgtcggtgccggtggtcgggaactgggagcagcgtttctgccgcctagcggacactttccagccggtcctggatcgggtgtacgacttcgaggtgcgggacgacgatgtgtggattgtcacgctgccaaaatgcggcaccacctggatgcaggagctggcctggctggtgatcaatgagtgtgattttgagacggccaagagcgtggatctcacgcatcgatcgcccttccttgaattcaatggcgtggttcccaatgtaccacacgacaccatagccgcagcgaatgccctgccatcgccgcgcctgatcaagtcccatctgcccgcctggatgctgccgaggcagatctggagcaagaggcccaagatcatttatgtgtaccgcaacccgaaggacgcggccatctcgtacttccaccactggcgtggaatggtgggctaccagggcaccaagtcggacttcatgcactcctttatcgacggctatgtgaacttcacgccctgctggccgcacatcctggacttttggcagctgcgccacgagcccaacatcttcttcaccagctacgagcggatgaagggccagctgggccaggttatctcggaggtggcccagttcctggagcgcagtgtcagccaggagcagatccagcagatgcagcgacacctctccttcgagagcatgcgcgacaatcccgcctgcaaccacgtcaaggagttcgagagcatgaaggcggctgccggcagggaggtggaggagttcaggttcgttcgccgcggagtcgtgggcagccacaaggatgagctcaccgccgacatcatccgggaattcgatctctggtccgatagtaatctgcgggatttcaaattgaacatggaatga

SEQ ID NO: 14 - DNA, 882 bp, Gallus gallus
  SULT1E1, pst-12
atggggaatgatgaggtgatcaggcaggacctgggctgcctttacgacatcccactctacaaatgctttgtggcaggctggccacaggtggaggccttccaagcccggcctgatgacctgctcatcgctacctaccccaagtcaggcacaacatggctgagcgagatcctggatgcaatctaccatgatggtgatttggagaagtgtcggcgggatgctatctataaccgggtgcccttcctggagatgaaagcccctgggatactgagcggtgtcgaacagctggagaaaatcccatccccacggctggtgaagactcacctgccagtccacctcctcccggcctccttccaggagaaagactgcaaggtaatctatatggctcgcaacgccaaggacgtcgtaatctcctactactacttctaccagatggccaagatccaccctgatcctggcacactgagcgagtttctgcaggcattcatggatgggaaagtggcctatgggtcctggtacaagcatgtgaagggctggtgggagaagaggcacgagaagcggcttctctacctcttctatgaggatatgaagaaggacccacggcgagagattcagaagatcctgcagttcctgggcaaggaggtggcagaagagacagtggcaaggatcctccaccacacctccttccaggagatgaagaagaaccctgccaccaactatgagacaatgcccactgaactgatggaccacagcctctccccattcatgcggaaaggcatctccggcgactgggcgaatcacttcactgtggcccagaatgagcgctttgaccagcactaccagcagcagatggcaggctcggatctctgcttccagatggaggca

SEQ ID NO: 15 - DNA, 888 bp, Gallus gallus
  SULT1B1-predicted -clone1, pst-16var (CBJ1149)
atggccacggtggacagatacctgcgtcagccctggagcgtggtgcacggcatccccatggtctgcgcctttgccccgaactgggagcgggtggacaatttccagagccgccctgaagatattgtggtggccaccttccccaagtctggcaccacttgggtcagtgagattgtggacatgatcctgcaaggcggtgacccagagaagtgcaaacgggatgccatcgtcaaccgggtacccatgttggagtttgttgcccctggggagatgccagcaggaacagaggtgctagctaccatgccctcaccccgtgtggtcaagagccacctgcctgcacatatcctgcccaaatccttctgggacaatggctgcaagataatctacgtgggtcgcaatgccaaggacgtggctgtctccttctactactttgacctgatgaataagtttgaacaacatccaggaacatgggaccagtacctggaggctttcatggctggcaaagtggcctatggctcttggtttgaccacgtgcggggctactgggagcgccggcaggagcaccccatcctctacctcttctatgaggacatgaaggaggatctccgccgggaggttgccaaggtggcccagttcctggggcgggagctgacggaagtggcactggacaccattgcccaccacacatcctttgaggccatgcgggacaatcccagcaccaattacagcagtgtgccctcccacctcatggaccatggggtctcccccttcatgcgcaaaggcatcaccggtgactggaagaaccacttcactgtggcccagagtgcacactttgaccagtactatgctcagaagatggcgggcactgacctgcgcttccgcacagaaatc

SEQ ID NO: 16 - DNA, 888 bp, Gallus gallus
  SULT1B1-predicted -clone2, pst-16var (CBJ1150)
atggccacggtggacagatacctgcatcagccctggagcgtggtgcacggcatccccatggtctgcgcctttgccccgaactgggagcgggtggacaatttccagagccgccctgaagatattgtggtggccaccttccccaagtctggcaccacttgggtcagtgagattgtggacatgatcctgcaaggcggtgacccagagaagtgcaaacgggatgccatcgtcaaccgggtacccatgttggagtttgttgcccctggggagatgccagcaggaacagaggtgctagctaccatgccctcaccccgtgtggtcaagagccacctgcctgcacatatcctgcccaaatccttctgggacaatggctgcaagataatctacgtgggtcgcaatgccaaggacgtggctgtctccttctactactttgacctgatgaataagtttgaacaacatccaggaacatgggaccagtacctggaggctttcatggctggcaaagtggcctatggctcttggtttgaccacgtgcggggctactgggagcgccggcaggagcaccccatcctctacctcttctatgaggacatgaaggaggatctccgccgggaggttgccaaggtggcccagttcctggggcgggagctgacggaagtggcactggacaccattgcccaccacacatcctttgaggccatgcgggacaatcccagcaccaattacagcagtgtgccctcccacctcatggaccatggggtctcccccttcatgcgcaaaggcatcaccggtgactggaagaaccacttcactgtggcccagagtgcacactttgaccagtactatgctcagaagatggcgggcactgacctgcgcttccgcacagaaatc

SEQ ID NO: 17 - DNA, 921 bp, Gallus gallus
  SULT1C1, pst-17var (CBJ1151)
atggccctggataaaatggaaaatttgagtctggaagagaatatgctccgatctgaaatgggtgaagtgcaaggtattcctgtcacgaaaccaacatgtgacatatgggatcaagtgtggaacttcaaagccagacctgatgatctgcttgtagcaacatacgcaaaagcaggtactacatggacacaggagatagtggatatgattcaacaaaatggagacattgagaagtgtagacgtgccagtacttacaaacgccatcctttccttgagtggtatatcccagattcttcacctctcggatactcaggcctgaagttagctgaagctatgccttccccgcgaacaatgaaaactcatctccccgtgcagctggtgcctccctccttctgggaacaaaactgcaagatcatctatgtggcaagaaatgcaaaagacaacctggtgtcatactaccatttccacagaatgaataaagtattacctgacccaggaaccatcgaggagttcacagagaaattcatgaatggagaagtgctctggggttcctggtatgaccacgtaaaaggatggtggaaggcaaaagacaagcaccgtattctctacctcttctatgaagatatgaaggagaatccaaagagagaaattcagaagattatgaagtttctggagaaggacttagacgaggaggttctcaacaagataatatataacacctcatttgagataatgaaagacaatcccatgacaaactacactaaggattttgtgggagtaatggatcactcagtttccccattcatgagaaaaggatctgtcggggactggaagaattatttcactgtggcactgaataagaagtttgatcaggattataagaagaaaatggctgatacctctctagtttttcgcatggaattg

SEQ ID NO: 18 - DNA, 1005 bp, Zostera marina
  KMZ74024.1, pst-42
atggacgaaagcaataccctgagcagcattagcaccgatgaacagcataaaagcatcgaagaaagcttcgccaaatacaaacagctgattgcaagcacctatccgcgttgtgatctgccgctgcagccgacactgtttcagcataaagatcatatgtggttcagcacctatgaactgatggttagcaccctggttgcacaggatcatctggttagccgtccgaccgatgtttttgttaccagctttccgaaaagcggcaccacctggctgaaaggtctggttagtagcattagtacccgtaacgatatggataaagttaatagcctggcaccgcatcatcagattccgtttctggaatttcatgtgtatccgtccgatgatcagatcatggatattgatagcctgcagagtccgcgttttctgagcacccatattccgtatccgagcctgccgaaaagcatgattgatagcggttgtcgtattgtgtatatttggcgtgatccgaaagcaacctttgtgagcttttttcattttagcaatcagatccgcaccaaatatggtacagcactgagcagtattgaagatcgtctgaaatggttttgtgatggctattgttggtttggtccgtattgggatcatgtgttaggttattggaacgccaaaaaaaacggtggcaatatcctgttcctgaaatatgaggatatgatggaagatccgtgcagccatatcaaaaccctggcagaatttatgggtgttccgtttaccgaacaagaagaaaaagaaggcgttatgcaggcaattattgcaagctgtagcttcgacaaaatgaaagattccgacgtgaacaaaatcggcaaaaccatgctgcatgttgaagtggataacaattccttttttcgcgaaggcaaaacgaatgactgggtgaattatctgacaccggaaatggttgaaaaactggatcgtattaccaaagaacgttttgcagataccgatctgattccgtaa

SEQ ID NO: 19 - DNA, 789 bp, Zostera marina
  KMZ73756.1, pst-48
atggatccgagcatgaatgaatatgaagtttttagcggtgtggtggatgatgatcagcaagaaaccattaaagaaagctatgccaaatatcgtcagctggttgcagcatttccgcgttgtaccagcctgagcagcgaatttgcaccgctggaagcagcattttttcagcacaaagaatacaaatggatcagccagtttagcaccatggttaattgtctggttgccgaaaaacattttgttggtcgtccgattgatgtttttgtgggtagctttccgaaaagcggcaccgtttggctgaaagatctggtgtataaaatcacccgtcatggtggtaatcaggatcataaaaatgatctgctgagtccgcatcagaaagttccgtttctggaactgcagatttatagcagcgaagatcatgtgctggatattgatagcctgccgagtccgcgtgttctgagcacccatattccgtatccgagtctgccgaccagtctgattgatagcggttgtccgattgtgtatatttggcgtgatccgaaagcagtttttgttagcgattggcacttcttcaacaaaatcattccgagcgaaccgggtacaaatctgccgagctttaccattaacaaaaagtttgaatgcttttgcaacggctactccatttttggtccgtattgggatcatgtgttaggttattggaacgccaaaaaaaacggcgtgaatatcctgctgatcaaatatgaagatctgatggcagatccggttgtgcagctgaaagcactggcagaattt

SEQ ID NO: 20 - DNA, 1017 bp, Escherichia coli
  cysP
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactga

SEQ ID NO: 21 - DNA, 834 bp, Escherichia coli
  cysU
atgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggcctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacggaagtgacgtcgctgatgatttttgtgcgcttacaggagtttgattacccggcagcgagcgcgattgcttcggtgatcctcgcggcatctctgctgctgctgttctcaattaacactctgcaaagtcgctttggtcggcgtgtggtaggtcattaa

SEQ ID NO: 22 - DNA, 876 bp, Escherichia coli
  cysW
atggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattga

SEQ ID NO: 23 - DNA, 1098 bp, Escherichia coli
  cysA
atgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 24 - DNA, 909 bp, Escherichia coli
  cysD
atggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaa

SEQ ID NO: 25 - DNA, 1428 bp, Escherichia coli
  cysN
atgaacaccgcacttgcacaacaaatcgccaatgaaggcggcgtcgaagcctggatgattgcgcaacaacataaaagcctgctgcgttttctgacctgtggtagcgtcgatgacggcaaaagtactctgattggtcgtctgctgcacgatacccgccaaatctacgaagatcagctctcatcgctgcataacgacagtaagcgtcacggcacccagggcgaaaagctggatctggctctgctggtggacggcctgcaagctgagcgcgaacagggcatcaccattgacgtggcctaccgctatttctctaccgagaagcgtaaatttattatcgccgacaccccagggcacgagcagtacacccgcaatatggcgactggcgcatcgacatgtgaactggcgatcttactgatcgatgcccgtaaaggcgtgctcgatcaaacccgtcgtcacagttttatctccacactgttggggatcaaacatctggtcgtggcgatcaacaaaatggatctggtggattacagtgaagagacgttcacccgtattcgtgaagattatttgacctttgccgggcagctgccgggtaatctggatatccgctttgtgccgctctctgcactggaaggcgacaacgtggcatcgcaaagtgaaagtatgccgtggtacagcggtccgacactgctcgaagtgctggaaaccgtggagatccagcgagtggtggatgctcagccaatgcgcttcccggtgcagtacgttaatcgcccgaatctcgattttcgtggttacgccggaacgctggcatccggtcgcgtggaagtcgggcaacgtgtcaaagtgctgccctctggtgtggaatcaaacgtcgcgcggatcgtgacttttgatggtgatcgcgaagaagcctttgccggagaagcgatcaccctggtgctgacggatgagatcgacatcagccgtggcgatctgctgctggcggcagacgaagcgttaccggcggtgcagagcgcgtcggtggatgtggtatggatggcggaacagccgctttctccagggcagagttacgacatcaaaattgccggtaagaagacgcgcgcgcgtgttgatggcattcgctatcaggttgatattaataaccttacccagcgtgaagttgaaaacctgccactgaatgggatcggcctcgtggatctcacttttgacgagccgctggtgttagatcgttatcaacaaaatccggtgacgggtgggctgatttttatcgatcgcctgagcaatgtgaccgtgggtgccggtatggtgcacgagccagttagccaggcaactgctgcgccatctgaattcagtgcattcgaactggaattgaatgctctggttcgtcgccactttccgcactggggcgcgcgcgatttgctgggggataaataa

SEQ ID NO: 26 - DNA, 606 bp, Escherichia coli
  cysC
atggcgctgcatgacgaaaacgtcgtctggcatagccatccggtcactgtgcaacaacgcgagctacaccacggtcatcgtggtgtagtgctgtggtttaccggcctctccgggtccggtaaatcaacggtcgccggggcgctggaggaggcgttacataaactcggcgtcagtacgtatctgctggatggcgacaatgttcgccacggattatgcagcgatctcggttttagcgatgccgatcgtaaagagaatatccgtcgcgtcggtgaagtggcgaatttgatggttgaagccggactggtggtgctgaccgcatttatctcgccacaccgcgccgaacgccagatggttcgcgaacgcgtaggagaagggcgctttatcgaagtgtttgtcgatacgccgctggcgatttgcgaagcccgcgatcccaaaggcttatataagaaagcgcgtgccggtgaactgcgcaactttacgggaatagattccgtttacgaagcgcctgaatcggcagaaattcatctcaatggtgaacaattagtaacaaatttggtacagcaattattagatctgttgagacagaacgatattatcagatcctga

SEQ ID NO: 27 - DNA, 741 bp, Escherichia coli
  cysQ
atgttagatcaagtatgccagcttgcacggaatgcaggcgatgccattatgcaggtctacgacgggacgaaaccgatggacgtcgtcagcaaagcggacaattctccggtaacggcagcggatattgccgctcacaccgttatcatggacggtttacgtacgctgacaccggatgttccggtcctttctgaagaagatcctcccggttgggaagtccgtcagcactggcagcgttactggctggtagacccgctggatggtactaaagagtttattaaacgtaatggcgaattcaccgttaacattgcgctcattgaccatggcaaaccgattttaggcgtggtgtatgcgccggtaatgaacgtaatgtacagcgcggcagaaggcaaagcgtggaaagaagagtgcggtgtgcgcaagcagattcaggtccgcgatgcgcgcccgccgctggtggtgatcagccgttcccatgcggatgcggagctgaaagagtatctgcaacagcttggcgaacatcagaccacgtccatcggctcttcgctgaaattctgcctggtggcggaaggacaggcgcagctgtacccgcgcttcggaccaacgaatatttgggacaccgccgctggacatgctgtagctgcagctgccggagcgcacgttcacgactggcagggtaaaccgctggattacactccgcgtgagtcgttcctgaatccggggttcagagtgtctatttactaa

SEQ ID NO: 28 - DNA, 1065 bp, Bacillus subtilis
  cysP
atggaattagccgctattttatttagcttgtttttcgcgatgaatattggtgcaagcggcgctgcagcttcgatgggggttgcttacggctccggagccatcaaaaagaaaacttacgctttgatcctatgcgcagtcggtgtctttgccggggcggtcattggcggaggcgaggttgtaaaaacaatcagctccggcattattcctgagcagacgattacactgacgattgtttgcatcattattggagctgctgccctgtcgctctttactgctaatctgctcggcattcctttgtcgacaagtgaagtaacagtcggcgcggttgtcggcgtgggagtggcttataaagtattgtttgtgaacaatcttctgatcattgtgtcattttgggtttttgtcccgctgttcgctttcggcttcacctatttcgtatctaagctgtttcgttattttaagattgaagtaaaatcttctaaaaagcaaaaaatcctcggcattgttttgttagttgccggatttttcgaagcattctctgccggcatgaataacgtggcgaatgctgttggcccgttagtagctgccggtgtgcttgatgtcggaaaaggaaccttgtatggaggagcttttgttgctcttggcgcactgttattaggccggcgagtgctagaaacaaacgggaagaaaattacgaggttctccaaaggggaaggcattctcttatccggtacaggagcgggactggtgatcatcagttctgtatttgggatgccggtgcccttggcgcaagtcacgtcatcgtctattatcggaataggcatggcgaaaaacggtccgaacgtgttccataaacaagttgttcaaacgatgctgaaggtatggatcgtatcgccgtttttatctttatcgatttcttatctgcttgtatccttgtttttaaaagcagattattattcgatctttatcatggtaagtgtgcttttagcggcgggcggtgccatcagcctgacaaaggccattcgtaaagagagacgctcagtccacgaacaaggcgggggaatctga

SEQ ID NO: 29 - protein, 285 aa, Homo sapiens
  SULT2A1, pst-50, NP_003158.2
MSDDFLWFEGIAFPTMGFRSETLRKVRDEFVIRDEDVIILTYPKSGTNWLAEILCLMHSKGDAKWIQSVPIWERSPWVESEIGYTALSETESPRLFSSHLPIQLFPKSFFSSKAKVIYLMRNPRDVLVSGYFFWKNMKFIKKPKSWEEYFEWFCQGTVLYGSWFDHIHGWMPMREEKNFLLLSYEELKQDTGRTIEKICQFLGKTLEPEELNLILKNSSFQSMKENKMSNYSLLSVDYVVDKAQLLRKGVSGDWKNHFTVAQAEDFDKLFQEKMADLPRELFPWE

SEQ ID NO: 30 - protein, 291 aa, Rattus norvegicus
  SULT1A1, pst-5, NP_114022.1
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL

SEQ ID NO: 31 - protein, 295 aa, Homo sapiens [sequence omitted]

SEQ ID NO: 32 - protein, 295 aa, Canis lupus familiaris
  SULT1A1, pst-13, NP_001003223.1
MEDIPDTSRPPLKYVKGIPLIKYFAEALESLQDFQAQPDDLLISTYPKSGTTWVSEILDMIYQDGDVEKCRRAPVFIRVPFLEFKAPGIPTGLEVLKDTPAPRLIKTHLPLALLPQTLLDQKVKVVYVARNAKDVAVSYYHFYRMAKVHPDPDTWDSFLEKFMAGEVSYGSWYQHVQEWWELSHTHPVLYLFYEDMKENPKREIQKILKFVGRSLPEETVDLIVQHTSFKEMKNNSMANYTTLSPDIMDHSISAFMRKGISGDWKTTFTVAQNERFDADYAKKMEGCGLSFRTQL

SEQ ID NO: 33 - protein, 295 aa, Sus scrofa domesticus
  SULT1A1, pst-14
MEPVQDTYRPPLEYVKGVPLIKYFAEALGPLESFQAWPDDVLISTYPKSGTTWVSEILDLIYQGGDLQKCQRAPIFVRVPFLEFKIPGCPTGFELLKDTPAPRLLKTHLPLALLPQTLLDQKVKVVYVARNAKDVAVSYYHFYRMAKVHPNPGTWDSFLEDFMAGEVSYGSWYQHVQEWWELRHTHPVLYLFYEDMKENPKREIQKILEFVGRSLPEETVEDIVQHTSFQEMKNNAMTNYRTLPSDLLDHSISAFMRKGITGDWKSTFTVAQNERFEADYAEKMAGCNLRFRSEL

SEQ ID NO: 34 - protein, 295 aa, Equus caballus ferus
  SULT1A1, pst-11, XP_001502088.1
MELIQDTSRPPLKYVKGVPLIKYFAEALGPLQSFQARPDDLLISTYPKSGTTWVSEILDMIYHGGDLEKCRRAPIFIRVPFLEFKAPEIPSGVEVLKDTPAPRLLKTHLPLSLLPQTLLDQKVKVVYLARNAKDVAVSYYHFYRMAKVHPDPGTWDSFLEKFMAGEVSYGSWYKHVQEWWELSHTHPVLYLFYEDMKENPKKEIQKILEFVGRSLPEETLDRIVQHTSFKEMKKNPMANYSTIPCDIMDHNISAFMRKGIAGDWKNTFTVAQNEHFDTDYAEKMAGCKLSFRSEV

SEQ ID NO: 35 - protein, 299 aa, Danio rerio
  SULT1ST1, pst-29, AAH56729.1
MDIPDFSSISSRPTIFEFEGISMINHFTENWEKVKNFQARPDDILIATYPKAGTTWVSYILDLLYFGENAPEEHTSQPIYMRVPFLESCFKVIASGTELADNMTTSPRLIKTHLPVQLIPKSFWEQNSRVVYVARNAKDNVVSYFHFDRMNIVEPDPGDWNTFLHRFMDGKSVFGPWYDHVNGYWEKKQTYSNLLYLFYEDLVEDTGREVDRLCSFLGLSTSVSDREKITKDVQFDAMKQNKMTNYSTLPVMDFKISPFMRKGKVGDWKNHFTVAQNEQFDEVYKEKMKNATVKFRTEI

SEQ ID NO: 36 - protein, 338 aa, Drosophila melanogaster
  dmST1, pst-6
MPQSSFFAKSVPFEQIDKLAISGGYSSIFASSKPSVPVVGNWEQRFCRLADTFQPVLDRVYDFEVRDDDVWIVTLPKCGTTWMQELAWLVINECDFETAKSVDLTHRSPFLEFNGVVPNVPHDTIAAANALPSPRLIKSHLPAWMLPRQIWSKRPKIIYVYRNPKDAAISYFHHWRGMVGYQGTKSDFMHSFIDGYVNFTPCWPHILDFWQLRHEPNIFFTSYERMKGQLGQVISEVAQFLERSVSGEQIQQMQRHLSFESMRDNPACNHVKEFESMKAAAGREVEEFRFVRRGVVGSHKDELTADIIREFDLWSDSNLRDFKLNMDDFANYSKFAST

SEQ ID NO: 37 - protein, 327 aa, Drosophila melanogaster
  dmST1 - clone2, pst-27
MPQSSFFAKSVPFEQIDKLAISGGYSSIFASSKPSVPVVGNWEQRFCRLADTFQPVLDRVYDFEVRDDDVWIVTLPKCGTTWMQELAWLVINECDFETAKSVDLTHRSPFLEFNGVVPNVPHDTIAAANALPSPRLIKSHLPAWMLPRQIWSKRPKIIYVYRNPKDAAISYFHHWRGMVGYQGTKSDFMHSFIDGYVNFTPCWPHILDFWQLRHEPNIFFTSYERMKGQLGQVISEVAQFLERSVSQEQIQQMQRHLSFESMRDNPACNHVKEFESMKAAAGREVEEFRFVRRGVVGSHKDELTADIIREFDLWSDSNLRDFKLNME

SEQ ID NO: 38 - protein, 294 aa, Gallus gallus
  SULT1E1, pst-12, XP_420616.4
MGNDEVIRQDLGCLYDIPLYKCFVAGWPQVEAFQARPDDLLIATYPKSGTTWLSEILDAIYHDGDLEKCRRDAIYNRVPFLEMKAPGILSGVEQLEKIPSPRLVKTHLPVHLLPASFQEKDCKVIYMARNAKDVVISYYYFYQMAKIHPDPGTLSEFLQAFMDGKVAYGSWYKHVKGWWEKRHEKRLLYLFYEDMKKDPRREIQKILQFLGKEVAEETVARILHHTSFQEMKKNPATNYETMPTELMDHSLSPFMRKGISGDWANHFTVAQNERFDQHYQQQMAGSDLCFQMEA

SEQ ID NO: 39 - protein, 296 aa, Gallus gallus
  SULT1B1-predicted -clone1, pst-16var (CBJ1149), NP_001299607.1
MATVDRYLRQPWSVVHGIPMVCAFAPNWERVDNFQSRPEDIVVATFPKSGTTWVSEIVDMILQGGDPEKCKRDAIVNRVPMLEFVAPGEMPAGTEVLATMPSPRVVKSHLPAHILPKSFWDNGCKIIYVGRNAKDVAVSFYYFDLMNKFEQHPGTWDQYLEAFMAGKVAYGSWFDHVRGYWERRQEHPILYLFYEDMKEDLRREVAKVAQFLGRELTEVALDTIAHHTSFEAMRDNPSTNYSSVPSHLMDHGVSPFMRKGITGDWKNHFTVAQSAHFDQYYAQKMAGTDLRFRTEI

SEQ ID NO: 40 - protein, 296 aa, Gallus gallus
  SULT1B1-predicted -clone2, pst-16var
MATVDRYLHQPWSVVHGIPMVCAFAPNWERVDNFQSRPEDIVVATFPKSGTTWVSEIVDMILQGGDPEKCKRDAIVNRVPMLEFVAPGEMPAGTEVLATMPSPRVVKSHLPAHILPKSFWDNGCKIIYVGRNAKDVAVSFYYFDLMNKFEQHPGTWDQYLEAFMAGKVAYGSWFDHVRGYWERRQEHPILYLFYEDMKEDLRREVAKVAQFLGRELTEVALDTIAHHTSFEAMRDNPSTNYSSVPSHLMDHGVSPFMRKGITGDWKNHFTVAQSAHFDQYYAQKMAGTDLRFRTEI

SEQ ID NO: 41 - protein, 307 aa, Gallus gallus
  SULT1C1, pst-17var (CBJ1151), NP_989932.1
MALDKMENLSLEENMLRSEMGEVQGIPVTKPTCDIWDQVWNFKARPDDLLVATYAKAGTTWTQEIVDMIQQNGDIEKCRRASTYKRHPFLEWYIPDSSPLGYSGLKLAEAMPSPRTMKTHLPVQLVPPSFWEQNCKIIYVARNAKDNLVSYYHFHRMNKVLPDPGTIEEFTEKFMNGEVLWGSWYDHVKGWWKAKDKHRILYLFYEDMKENPKREIQKIMKFLEKDLDEEVLNKIIYNTSFEIMKDNPMTNYTKDFVGVMDHSVSPFMRKGSVGDWKNYFTVALNKKFDQDYKKKMADTSLVFRMEL

SEQ ID NO: 42 - protein, 334 aa, Zostera marina
  KMZ74024.1, pst-42
MDESNTLSSISTDEQHKSIEESFAKYKQLIASTYPRCDLPLQPTLFQHKDHMWFSTYELMVSTLVAQDHLVSRPTDVFVTSFPKSGTTWLKGLVSSISTRNDMDKVNSLAPHHQIPFLEFHVYPSDDQIMDIDSLQSPRFLSTHIPYPSLPKSMIDSGCRIVYIWRDPKATFVSFFHFSNQIRTKYGTALSSIEDRLKWFCDGYCWFGPYWDHVLGYWNAKKNGGNILFLKYEDMMEDPCSHIKTLAEFMGVPFTEQEEKEGVMQAIIASCSFDKMKDSDVNKIGKTMLHVEVDNNSFFREGKTNDWVNYLTPEMVEKLDRITKERFADTDLIP

SEQ ID NO: 43 - protein, 284 aa, Zostera marina
  KMZ73756.1, pst-48
MDFSVSHHRKLFSGTKVFRGSSHRCVYRLTPQIRDGLVEALQKKVGSSDEAKLTKFSLKIVFVVKDLVYKIKSRGGNQDHKKDLLSPHQKVPFLELQVYASEDHVLDIDSLPSPRLLSTHIPYPSLPTSLIDSGCSIVYIWRDPKAFECFCNGYSIFGPYWDHVLGYWNAKKNGANILLIKYEDLMVDPVAQLKILAEFLKLPFTEEEEEDNIIHGIITACSFQMVKDSKMYESGNTELLQVQVNNTTFLRERKTNDWENYLILKMAERLNLITVQRFADTDLI

SEQ ID NO: 44 - protein, 338 aa, Escherichia coli
  cysP, NP_416920.1
MAVNLLKKNSLALVASLLLAGHVQATELLNSSYDVSRELFAALNPPFEQQWAKDNGGDKLTIKQSHAGSSKQALAILQGLKADVVTYNQVTDVQILHDKGKLIPADWQSRLPNNSSPFYSTMGFLVRKGNPKNIHDWNDLVRSDVKLIFPNPKTSGNARYTYLAAWGAADKADGGDKGKTEQFMTQFLKNVEVFDTGGRGATTTFAERGLGDVLISFESEVNNIRKQYEAQGFEVVIPKTNILAEFPVAWVDKNVQANGTEKAAKAYLNWLYSPQAQTIITDYYYRVNNPEVMDKLKDKFPQTELFRVEDKFGSWPEVMKTHFTSGGELDKLLAAGRN

SEQ ID NO: 45 - protein, 277 aa, Escherichia coli
  cysU, NP_416919.1
MFAVSSRRVLPGFTLSLGTSLLFVCLILLLPLSALVMQLAQMSWAQYWEVITNPQVVAAYKVTLLSAFVASIFNGVFGLLMAWILTRYRFPGRTLLDALMDLPFALPTAVAGLTLASLFSVNGFYGEWLAKFDIKVTYTWLGIAVAMAFTSIPFVVRTVQPVLEELGPEYEEAAETLGATRWQSFCKVVLPELSPALVAGVALSFTRSLGEFGAVIFIAGNIAWKTEVTSLMIFVRLQEFDYPAASAIASVILAASLLLLFSINTLQSRFGRRVVGH

SEQ ID NO: 46 - protein, 291 aa, Escherichia coli
  cysW, YP_026168.2
MAEVTQLKRYDARPINWGKWFLIGIGMLVSAFILLVPMIYIFVQAFSKGLMPVLQNLADPDMLHAIWLTVMIALIAVPVNLVFGILLAWLVTRFNFPGRQLLLTLLDIPFAVSPVVAGLVYLLFYGSNGPLGGWLDEHNLQIMFSWPGMVLVTIFVTCPFVVRELVPVMLSQGSQEDEAAILLGASGWQMFRRVTLPNIRWALLYGVVLTNARAIGEFGAVSVVSGSIRGETLSLPLQIELLEQDYNTVGSFTAAALLTLMAIITLFLKSMLQWRLENQEKRAQQEEHHEH

SEQ ID NO: 47 - protein, 365 aa, Escherichia coli
  cysA, NP_416917.1
MSIEIANIKKSFGRTQVLNDISLDIPSGQMVALLGPSGSGKTTLLRIIAGLEHQTSGHIRFHGTDVSRLHARDRKVGFVFQHYALFRHMTVFDNIAFGLTVLPRRERPNAAAIKAKVTKLLEMVQLAHLADRYPAQLSGGQKQRVALARALAVEPQILLLDEPFGALDAQVRKELRRWLRQLHEELKFTSVFVTHDQEEATEVADRVVVMSQGNIEQADAPDQVWREPATRFVLEFMGEVNRLQGTIRGGQFHVGAHRWPLGYTPAYQGPVDLFLRPWEVDISRRTSLDSPLPVQVLEASPKGHYTQLVVQPLGWYNEPLTVVMHGDDAPQRGERLFVGLQHARLYNGDERIETRDEELALAQSA

SEQ ID NO: 48 - protein, 302 aa, Escherichia coli
  cysD, NP_417232.1
MDQIRLTHLRQLEAESIHIIREVAAEFSNPVMLYSIGKDSSVMLHLARKAFYPGTLPFPLLHVDTGWKFREMYEFRDRTAKAYGCELLVHKNPEGVAMGINPFVHGSAKHTDIMKTEGLKQALNKYGFDAAFGGARRDEEKSRAKERIYSFRDRFHRWDPKNQRPELWHNYNGQINKGESIRVFPLSNWTEQDIWQYIWLENIDIVPLYLAAERPVLERDGMLMMIDDNRIDLQPGEVIKKRMVRFRTLGCWPLTGAVESNAQTLPEIIEEMLVSTTSERQGRVIDRDQAGSMELKKRQGYF

SEQ ID NO: 49 - protein, 475 aa, Escherichia coli
  cysN, NP_417231.1
MNTALAQQIANEGGVEAWMIAQQHKSLLRFLTCGSVDDGKSTLIGRLLHDTRQIYEDQLSSLHNDSKRHGTQGEKLDLALLVDGLQAEREQGITIDVAYRYFSTEKRKFIIADTPGHEQYTRNMATGASTCELAILLIDARKGVLDQTRRHSFISTLLGIKHLVVAINKMDLVDYSEETFTRIREDYLTFAGQLPGNLDIRFVPLSALEGDNVASQSESMPWYSGPTLLEVLETVEIQRVVDAQPMRFPVQYVNRPNLDFRGYAGTLASGRVEVGQRVKVLPSGVESNVARIVTFDGDREEAFAGEAITLVLTDEIDISRGDLLLAADEALPAVQSASVDVVWMAEQPLSPGQSYDIKIAGKKTRARVDGIRYQVDINNLTQREVENLPLNGIGLVDLTFDEPLVLDRYQQNPVTGGLIFIDRLSNVTVGAGMVHEPVSQATAAPSEFSAFELELNALVRRHFPHWGARDLLGDK

SEQ ID NO: 50 - protein, 201 aa, Escherichia coli
  cysC, NP_417230.1
MALHDENVVWHSHPVTVQQRELHHGHRGVVLWFTGLSGSGKSTVAGALEEALHKLGVSTYLLDGDNVRHGLCSDLGFSDADRKENIRRVGEVANLMVEAGLVVLTAFISPHRAERQMVRERVGEGRFIEVFVDTPLAICEARDPKGLYKKARAGELRNFTGIDSVYEAPESAEIHLNGEQLVTNLVQQLLDLLRQNDIIRS

SEQ ID NO: 51 - protein, 246 aa, Escherichia coli
  NP_418635.1
MLDQVCQLARNAGDAIMQVYDGTKPMDVVSKADNSPVTAADIAAHTVIMDGLRTLTPDVPVLSEEDPPGWEVRQHWQRYWLVDPLDGTKEFIKRNGEFTVNIALIDHGKPILGVVYAPVMNVMYSAAEGKAWKEECGVRKQIQVRDARPPLVVISRSHADAELKEYLQQLGEHQTTSIGSSLKFCLVAEGQAQLYPRFGPTNIWDTAAGHAVAAAAGAHVHDWQGKPLDYTPRESFLNPGFRVSIY

SEQ ID NO: 52 - protein, 354 aa, Bacillus subtilis
  cysP, NP_389441.1
MELAAILFSLFFAMNIGASGAAASMGVAYGSGAIKKKTYALILCAVGVFAGAVIGGGEVVKTISSGIIPEQTITLTIVCIIIGAAALSLFTANLLGIPLSTSEVTVGAVVGVGVAYKVLFVNNLLIIVSFWVFVPLFAFGFTYFVSKLFRYFKIEVKSSKKQKILGIVLLVAGFFEAFSAGMNNVANAVGPLVAAGVLDVGKGTLYGGAFVALGALLLGRRVLETNGKKITRFSKGEGILLSGTGAGLVIISSVFGMPVPLAQVTSSSIIGIGMAKNGPNVFHKQVVQTMLKVWIVSPFLSLSISYLLVSLFLKADYYSIFIMVSVLLAAGGAISLTKAIRKERRSVHEQGGGI

SEQ ID NO: 53 - DNA, 1986 bp, Escherichia coli
  yjcS, NC_000913.3c4306597-4304612 Escherichia coli str. K-12 substr. MG1655
atgaataactctcggttattccgtttgagcaggattgttattgcgttaactgccgccagcggcatgatggtaaataccgctaacgcgaaagaggaagcgaaagccgccactcaatatacccaacaggttaatcagaattacgccaaatcattaccgtttagcgatcgtcaggattttgacgatgcccagcgtggatttatcgccccgctgctggatgaaggtattctgcgtgatgcgaacggtaaagtttactaccgcgcggacgattacaaatttgatattaatgccgcagcgccggaaaccgtaaaccccagcctgtggcgtcagtcgcaaatcaacggtatttctggcctgttcaaagtcaccgataaaatgtatcaggtgcgcggccaggatatctctaacattacgttcgttgagggcgagaaaggcattattgttatcgacccgctggtgacgccgcctgccgcaaaagccgcacttgacctttacttccagcatcgtccgcaaaaaccgattgttgccgttatctacactcacagccacaccgaccactatggtggcgtgaaaggcattatctctgaagccgatgttaaatccggcaaagttcaggtgattgcccctgcaggctttatggacgaagccatcagcgaaaacgtgctggcgggtaacatcatgagccgccgtgcgctctactcttacggtctgttactgccgcacaacgcgcaaggcaatgtgggtaatggccttggcgtgacgctggcaacgggcgacccgagcattattgcaccgacgaaaactatcgtcagaactggcgagaagatgattatcgacggcctggagtttgacttcctgatgaccccaggtagcgaagcgccagccgaaatgcacttctatattccggccctgaaagccctgtgtaccgccgagaacgccacgcataccctgcacaacttctacactctgcgcggcgcgaaaacccgcgacaccagcaagtggaccgagtatctgaacgaaacgctggatatgtggggtaacgacgcggaagtgctgtttatgccgcacacctggccggtctggggcaataagcatatcaatgattatattggtaaataccgcgataccatcaagtacattcacgaccagaccctgcacctggcgaaccagggctacaccatgaatgaaatcggcgacatgattaagctgccgcctgcacttgccaataactgggccagccgcggctattacggttctgtcagccacaacgcccgcgcggtgtataacttctatcttggctattacgacggtaacccggctaacctgcatccgtatggtcaggtggagatgggtaaacgttacgtgcaggcgctgggcggttctgcccgtgtcatcaacctggcgcaagaagcgaacaagcaaggtgattaccgctggtcggcagaactgctgaaacaggtgattgccgccaacccgggtgaccaggtcgcgaagaatctgcaagcgaataactttgaacagctgggctatcaggccgagtccgccacatggcgcggtttctacctgaccggcgcgaaagagctgcgcgaaggggtgcataagttcagccacggcaccaccggttccccggacaccattcgcgggatgtcggtcgaaatgctgttcgactttatggccgttcgcctcgatagcgcgaaagctgcgggtaaaaatatcagcctgaacttcaatatgagcaacggcgataacctcaacctgacgctgaacgatagcgtgcttaactaccggaaaacgctgcaaccgcaagccgacgcctctttctacatcagccgtgaagatctgcacgccgtgctgaccggacaagccaaaatggcggatctggtaaaagcgaagaaagccaaaattattggcaatggcgcgaaactggaagaaattatcgcctgtctggataatttcgatttgtgggtgaatatcgtaaccccaaattaa

SEQ ID NO: 54 - DNA, 1656 bp, Escherichia coli
  aslA, NC_000913.3c3986007-3984352 Escherichia coli str. K-12 substr. MG1655
atggaattttcgttttcacccaaacgtcttgttgttgctgtcgccgccgctcttcctctcatggccagcgcagcagataccccgtcaactgccaccgcacgcaaaggctttgccggatacgatcacccaaaccagtatctggttaaaccggcgaccactattgccgacaatatgatgccagtaatgcagcatccggcgcaggataaagaaacccagcagaagctggcagaacttgagaaaaaaaccggtaagaaaccgaatgtggttgttttcttgctggacgatgtgggctggatggacgtcggttttaacggtggcggcgtggcggtgggtaaccctacaccagatatcgacgccgttgccagccaggggctgattttaacttcggcgtattctcaaccaagctcttccccaacccgcgccaccattctcaccggacaatactccatccaccacggcattctgatgccgccaatgtacgggcaaccgggcgggctgcaagggttaaccacgctgccgcagttgctgcacgatcagggctacgtcactcaggccatcggaaaatggcatatgggggaaaacaaagagtcgcagccgcagaacgttggctttgatgatttccgtggctttaactcggtgtctgatatgtacaccgaatggcgcgacgttcacgtcaatccggaagtggccctgagtccggaccgttctgaatacatcaagcaattaccgttcagcaaagatgacgttcatgcggtgcgcggcggcgaacaacaggccattgccgacattacgccgaaatatatggaagatctggatcaacgctggatggactatggcgttaagttcctcgacaagatggcgaagagcgataaaccattcttcctctactacggcactcgtggctgccacttcgataactacccaaatgcgaaatatgcgggtagctctccggcacgcacctcgtatggcgactgcatggtggagatgaacgatgtgttcgctaatctgtataaaacactggagaaaaacggtcagcttgataacacgctgatcgtctttacctccgataacggaccggaagccgaagtaccgccgcacggacgcaccccgttccgtggtgcgaaaggttcgacctgggaaggcggcgttcgcgtaccgactttcgtttactggaaagggatgatccaaccgcgtaaatctgacggtattgtcgatctggcagatctcttccctaccgcgctggatctggcagggcatcctggagcgaaagtggcgaatttagtgccgaaaaccacctttatcgatggtgtggaccagacatccttcttcctgggaacaaatggtcagtctaaccgtaaggccgagcactacttcctcaacggtaaactcgctgctgtgcgtatggatgagttcaagtatcacgtcctgattcagcaaccttacgcttatacccagagcggatatcagggtggattcaccggcacagtaatgcaaacggcgggatcgtcggtgtttaacctctacaccgatccgcaggaaagcgactccatcggcgtgcgccatattccgatgggtgtaccgctacagaccgaaatgcacgcgtatatggagatcctgaaaaaatatccaccacgcgcgcagattaaatctgactaa

SEQ ID NO: 55 - DNA, 1683 bp, Escherichia coli
  ydeN, NC_000913.3c1582524-1580842 Escherichia coli str. K-12 substr. MG1655
atgaagtctgcattaaagaaaagtgtcgtaagtacctcgatatctttgatactggcatctggtatggctgcatttgctgctcatgcggcagatgatgtaaagctgaaagcaaccaaaacaaacgttgctttctcagactttacgccgacagaatacagtaccaaaggaaagccaaatattatcgtactgaccatggatgatcttggttatggacaacttccttttgataagggatcttttgacccaaaaacaatggaaaatcgtgaagttgtcgatacctacaaaatagggatagataaagccattgaagctgcacaaaaatcaacgccgacgctcctttcattaatggatgaaggcgtacgttttactaacggctatgtggcacacggtgtttccggcccctcccgcgccgcaataatgaccggtcgagctcccgcccgctttggtgtctattccaataccgatgctcaggatggtattccgctaacagaaactttcttgcctgaattattccagaatcatggttattacactgcagcagtaggtaaatggcacttgtcaaaaatcagtaatgtgccggtaccggaagataaacaaacgcgtgactatcatgacaacttcaccacattttctgcggaagaatggcaacctcaaaaccgtggctttgattactttatgggattccacgctgcaggaacggcatattacaactccccttcactgttcaaaaatcgtgaacgtgtccccgcaaaaggttatatcagcgatcagttaaccgatgaggcaattggcgttgttgatcgtgccaaaacacttgaccagccttttatgctttacctggcttataatgctccgcacctgccaaatgataatcctgcaccggatcaatatcagaagcaatttaataccggtagtcaaacagcagataactactacgcttccgtttattctgttgatcagggtgtaaaacgcattctcgaacaactgaagaaaaacggacagtatgacaatacaattattctctttacctccgataatggtgcggttatcgatggtcctctgccgctgaacggggcgcaaaaaggctataagagtcagacctatcctggcggtactcacaccccaatgtttatgtggtggaaaggaaaacttcaacccggtaattatgacaagctgatttccgcaatggatttctacccgacagctcttgatgcagccgatatcagcattccaaaagaccttaagctggatggcgtttccttgctgccctggttgcaagataagaaacaaggcgagccacataaaaatctgacctggataacctcttattctcactggtttgacgaggaaaatattccattctgggataattaccacaaatttgttcgccatcagtcagacgattacccgcataaccccaacactgaggacttaagccaattctcttatacggtgagaaataacgattattcgcttgtctatacagtagaaaacaatcagttaggtctctacaaactgacggatctacagcaaaaagataaccttgccgccgccaatccgcaggtcgttaaagagatgcaaggcgtggtaagagagtttatcgacagcagccagccaccgcttagcgaggtaaatcaggagaagtttaacaatatcaagaaagcactaagcgaagcgaaataa

SEQ ID NO: 56 - DNA, 1494 bp, Escherichia coli
  yidJ, NC_000913.3c3858404-3856911 Escherichia coli str. K-12 substr. MG1655
atgaaacgccccaattttctgttcgtcatgaccgatacccaggccaccaatatggtcggttgctatagcggtaaaccgctgaatacgcaaaatattgatagtctggcggcggaaggtattcgctttaattccgcctacacctgttcaccggtttgtacgcctgcacgggccggactatttaccggtatctacgctaaccagtccggcccgtggaccaacaacgtcgcgccaggcaaaaacatctccactatggggcgctactttaaggatgccggctatcacacctgttacatcggcaaatggcatctcgacggtcatgactatttcggcactggcgagtgtccgccggagtgggacgctgattactggttcgatggggcgaactatcttagcgaactgacggaaaaagagattagcctgtggcgcaatggcctaaacagcgtcgaagatttacaggcgaaccatatcgacgaaaccttcacctgggcgcatcgtatcagcaatcgggcggtggattttctgcaacagcctgcgcgcgcagacgagcctttcctgatggtggtttcgtatgatgagccgcatcacccgttcacctgtccggtggagtatttagagaaatacgctgatttttactacgagctgggcgagaaagcacaggatgacctggctaacaaaccggaacatcaccgcttatgggcgcaggcgatgccatcgccagtcggtgatgacgggctttatcaccatccgctctattttgcctgtaatgactttgttgatgaccaaatcggacgggtcatcaatgccttaacgccagagcaacgtgaaaatacgtgggttatttatacctccgatcacggcgaaatgatgggcgcacataagctgatcagtaaaggggcggcgatgtatgacgacatcacccgcattccgctgatcatccgttcgccgcaaggggagcggcgacaggtcgatacgccagtcagtcatatcgatttactgccgacaatgatggcgctggcagatattgaaaaaccagagattctgccgggggaaaatatccttgccgtgaaagagccacgcggcgtgatggtggaatttaaccgctacgagattgagcatgacagctttggcggttttattccggtgcgttgctgggtgacggatgactttaaactggtactcaacctcttcaccagtgatgaactttacgatcgccgtaatgacccaaatgaaatgcataacctgatcgatgatatccgttttgcagacgttcgcagcaaaatgcatgacgccttattggattacatggacaaaattcgcgatccgttccgcagttaccaatggagtctgcgtccgtggcgtaaagatgcacggccgcgctggatgggggcgtttcgtccacgtccacaagatggctattcgccagttgtacgcgactatgacaccggcctaccgacacaaggggtgaaggtggaggagaaaaaacagaagttctga

SEQ ID NO: 57 - DNA, 1158 bp, Escherichia coli
  ydeM, NC_000913.3c1580790-1579633 Escherichia coli str. K-12 substr. MG1655
atgcacgttacagccaagccctccagttttcaatgtaatctcaaatgtgattactgtttttaccttgaaaaagagtcgcagtttactcatgaaaaatggatggatgacagcactctgaaagagttcatcaaacaatatatcgcagcgtctggcaatcaggtctattttacctggcaaggcggtgaacccactctggctggcctggattttttccgtaaagttattcactatcaacaacgctatgcaggccaaaaacgtatttttaatgcattacaaacgaatggcattttattgaataatgaatggtgtgcctttctcaaagaacatgaatttctggttggtatctcgatcgatggcccccaggagttacatgaccgttacagacgcagtaattcaggtaacggtacttttgcaaaagtgatagcagccatcgagcgtctgaaatcatatcaagtagagtttaatacgttaaccgtcattaataacgttaatgtccattaccctcttgaggtttatcattttttaaaatctatcggcagtaaacatatgcaatttatcgaattgctagaaaccgggacgccgaatattgatttcagtggtcatagtgagaacacattccgtatcattgatttttctgtgcctcccacggcttatggcaagtttatgtcaaccatttttatgcaatgggttaaaaacgatgtgggtgaaattttcatccgtcagtttgaaagctttgtcagccgttttttggggaatgggcataccagttgtattttccaggagtcctgcaaggataatctggttgttgaaagtaatggagacatttacgaatgcgaccattttgtctatccacagtacaaaattggaaacattaataaatctgaactcaaaacgatgaacagtgtacaactgacagcgcaaaaaaaacggattccagcgaaatgtcagcaatgtgcatataaacctatctgcaatggcggttgtcctaagcatcgtattactaaagtaaacaatgagactgtttcttatttttgcgaaggttataaaatccttttttcaaccatggtaccttatatgaacgccatggtagagttagctaagaacagagtaccgctttaccacattatggatgttgcaaaacaaatggagaataattaa

SEQ ID NO: 58 - DNA, 1236 bp, Escherichia coli
  aslB, NC_000913.33982958-3984193 Escherichia coli str. K-12 substr. MG1655
atgctgcaacaggttccaacgcgtgcttttcatgtgatggcgaaaccgagcggttccgattgtaatctgaactgtgactactgtttttatctcgaaaaacaatccctttaccgcgaaaagccagtcacgcatatggacgatgacacgctggaagcgtatgtccgtcactatatcgctgccagcgaaccgcaaaacgaagtggcttttacctggcagggcggcgaaccaacgctactcgggctggcgttttaccgccgtgccgtagcgctacaggcgaaatatggtgctggcaggaagataagtaacagcttccagactaacggcgtgctgctggatgacgaatggtgcgcgtttctcgcggagcatcattttcttgttggtttatcgctggatggcccgcctgagatccacaatcaatatcgcgtgactaaaggtggcagacccacgcataagctggtgatgcgtgccctgacgctcctgcaaaaacatcatgtcgactataacgtgctggtctgcgttaatcgcaccagcgcgcagcaaccgttgcaggtatatgattttttgtgcgatgcgggagtcgaattcatccagtttattccggtggtcgagcgcctggctgatgaaacaactgcccgcgatggacttaagttacatgcgcctggtgatattcagggtgagctaacggaatggtcggtgcgccccgaggagttcggtgagtttctggtggcgatattcgaccactggatcaaacgcgacgtcggcaagattttcgtgatgaatatcgaatgggcgtttgccaattttgtcggtgcgccgggtgcggtttgccatcatcagccaacctgtgggcgctcggtgattgttgagcacaacggcgacgtttacgcctgtgatcactatgtttatccgcaatatcggctggggaatatgcaccagcaaacaattgcagaaatgatcgattccccgcaacagcaggcgtttggtgaagataaatttaagcagttaccggcgcagtgtcgcagttgtaacgtgttaaaagcgtgctggggaggctgcccgaaacaccgcttcatgctcgatgccagcggcaaaccgggactgaattatttgtgtgccgggtatcagcgttatttccgccatctaccgccatatcttaaagcaatggctgatttgctggcgcacggtcgcccggccagcgacattatgcatgcgcatttgctggtggtgagtaagtag

SEQ ID NO: 59 - DNA, 768 bp, Escherichia coli
  hdhA, NC_000913.3c1698040-1697273 Escherichia coli str. K-12 substr. MG1655
gtgtttaattctgacaacctgagactcgacggaaaatgcgccatcatcacaggtgcgggtgcaggtattggtaaagaaatcgccattacattcgcgacagctggcgcatctgtggtggtcagtgatattaacgccgacgcagctaaccatgttgtagacgaaattcaacaactgggtggtcaggcatttgcctgccgttgtgatattacttccgaacaggaactctctgcactggcagactttgctatcagtaagctgggtaaagttgatattctggttaacaacgccggtggcggtggacctaaaccgtttgatatgccaatggcggattttcgccgtgcttatgaactgaatgtgttttcttttttccatctgtcacaacttgttgcgccagaaatggaaaaaaatggcggtggcgttattctgaccatcacttctatggcggcagaaaataaaaatataaacatgacttcctatgcatcatctaaagctgcggccagtcatctggtcagaaatatggcgtttgacctgggtgaaaaaaatattcgggtaaatggcattgcgccgggggcaatattaaccgatgccctgaaatccgttattacaccagaaattgaacaaaaaatgttacagcacacgccgatcagacgtctgggccaaccgcaagatattgctaacgcagcgctgttcctttgctcgcctgctgcgagctgggtaagcggacaaattctcaccgtctccggtggtggggtacaggagctcaattaa

SEQ ID NO: 60 - DNA, 87 bp, Artificial Sequence
  Regulatory T7 Inducible promoter sequence
taatacgactcactataggggaattgtgagcggataacaattcccctctagaaataattttgtttaactttaagaaggagatatacc

SEQ ID NO: 61 - DNA, 82 bp, Artificial Sequence
  Regulatory Pmic7 Constitutive promoter sequence
tgcttgactcgtcgttcctcctacgtgtataattggacccctctagaaataattttgtttaactttaagaaggagatatacc

SEQ ID NO: 62 - DNA, 35 bp, Artificial Sequence
  BBa_J23110 promoter sequence
tttacggctagctcagtcctaggtacaatgctagc

SEQ ID NO: 63 - DNA, 68 bp, Artificial Sequence
  pMUT backbone reverse primer
aggggtccaattatacacgtaggaggaacgacgagtcaagcaatttcgcgggatcgagatcgatctcg

SEQ ID NO: 64 - DNA, 858 bp, Artificial Sequence
  S. boulardii codon optimized SULT2A1
atgtctgacgacttcttgtggtttgaaggcattgcgtttcctacaatgggcttcaggtcagagactcttagaaaggttagggatgaatttgttattagagatgaagatgtcatcatactgacatatcctaaaagtgggactaactggcttgctgagatcttgtgcttgatgcatagcaagggggatgctaagtggattcagagcgttcctatctgggaacgttctccttgggtcgagtctgaaatcggatacactgcattatctgagacagagagcccgcgtttgttctcttcccacctaccaattcaacttttccctaagtcttttttttcatctaaggcgaaagttatttatctgatgcgtaacccgcgtgacgtgttggtcagtggttattttttctggaaaaacatgaagttcatcaagaaaccgaagagctgggaagagtatttcgagtggttctgtcagggtacagtcctgtacggatcatggttcgaccatattcacgggtggatgcctatgagagaagaaaagaattttttactgttgagctacgaagagctaaaacaagacaccgggaggacgatagagaagatttgccagtttttaggtaagacactagagcccgaagaactaaatctaatcctgaaaaatagttccttccagagtatgaaagagaacaagatgagtaactacagtttattaagcgtagattacgtcgtggataaggctcagctacttaggaagggggtctcaggtgactggaagaaccacttcactgtcgcccaggcggaggacttcgacaaactgtttcaggaaaaaatggccgacctaccgagggaactattcccatgggaataa

SEQ ID NO: 65 - DNA, 50 bp, Artificial Sequence
  SULT2A1-fw primer
tcgtcatccttgtaatccatcgatactagtcaacggaatgcgtgcgatcg

SEQ ID NO: 66 - DNA, 30 bp, Artificial Sequence
  SULT2A1 rv primer
ctaactccttccttttcggttagagcggat

SEQ ID NO: 67 - DNA, 23 bp, Artificial Sequence
  pCfB2055-fw primer
atccgctctaaccgaaaaggaag

SEQ ID NO: 68 - DNA, 18 bp, Artificial Sequence
  pCfB2055 rv primer
gaatgcacgcgatcgcac

SEQ ID NO: 69 - DNA, 735 bp, Escherichia coli
  cysH, NC_000913.3c2888312-2887578
atgtccaaactcgatctaaacgccctgaacgaactgccgaaggtagatcgcattctggcgctggcggaaactaacgccgaactggaaaaactggacgctgaaggccgcgtagcctgggcgctggataatctgcccggtgaatatgtactttcttccagctttggcattcaggcggcggtgagcctgcatctggtgaatcaaattcgcccggatattccggtgatcctcaccgatacgggttacttgttcccggaaacctaccgctttattgacgagttaacggacaaactcaagctcaacctgaaagtgtaccgtgctaccgaaagcgcagcctggcaggaagcacgctacggaaaactgtgggagcagggcgttgaaggcattgaaaagtacaatgacatcaacaaagtcgaaccgatgaaccgggctctgaaagaactgaatgcgcaaacctggtttgctggcctgcgccgtgaacaatccggcagtcgtgccaatttaccggtgctggcaattcagcgtggcgtatttaaagtgctgccgattatcgactgggataaccgaactatttatcagtacctgcaaaaacatggcctgaaatatcacccattatgggatgaaggatatttatcggtgggcgatacccatacaacccgtaaatgggaacccggcatggcggaagaagaaacgcgtttctttggcttaaaaagggaatgtgggttacacgaagggtaa

SEQ ID NO: 70 - DNA, 741 bp, Escherichia coli; identical to SEQ ID NO: 27
  cysQ, NC_000913.34436755-4437495

SEQ ID NO: 71 - DNA, 3150 bp, Escherichia coli
  acrB, NC_000913.3c484403-481254
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccccggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggcgttatcaacaccgatggcaccatgacgcaggaggatatctccgactacgtggcggcgaatatgaaagatgccatcagccgtacgtcgggcgtgggtgatgttcagttgttcggttcacagtacgcgatgcgtatctggatgaacccgaatgagctgaacaaattccagctaacgccggttgatgtcattaccgccatcaaagcgcagaacgcccaggttgcggcgggtcagctcggtggtacgccgccggtgaaaggccaacagcttaacgcctctattattgctcagacgcgtctgacctctactgaagagttcggcaaaatcctgctgaaagtgaatcaggatggttcccgcgtgctgctgcgtgacgtcgcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccatacgacaccacgccgttcgtgaaaatctctattcacgaagtggttaaaacgctggtcgaagcgatcatcctcgtgttcctggttatgtatctgttcctgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgacgccatcgttgtggtagaaaacgttgagcgtgttatggcggaagaaggtttgccgccaaaagaagctacccgtaagtcgatggggcagattcagggcgctctggtcggtatcgcgatggtactgtcggcggtattcgtaccgatggccttctttggcggttctactggtgctatctatcgtcagttctctattaccattgtttcagcaatggcgctgtcggtactggtggcgttgatcctgactccagctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttacctggtgctgtatctgatcatcgtggtcggcatggcctatctgttcgtgcgtctgccaagctccttcttgccagatgaggaccagggcgtgtttatgaccatggttcagctgccagcaggtgcaacgcaggaacgtacacagaaagtgctcaatgaggtaacgcattactatctgaccaaagaaaagaacaacgttgagtcggtgttcgccgttaacggcttcggctttgcgggacgtggtcagaataccggtattgcgttcgtttccttgaaggactgggccgatcgtccgggcgaagaaaacaaagttgaagcgattaccatgcgtgcaacacgcgctttctcgcaaatcaaagatgcgatggttttcgcctttaacctgcccgcaatcgtggaactgggtactgcaaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggcagctatgtgaacgactttatcgaccgcggtcgtgtgaagaaagtttatgtcatgtcagaagcgaaataccgtatgctgccggatgatatcggcgactggtatgttcgtgctgctgatggtcagatggtgccattctcggcgttctcctcttctcgttgggagtacggttcgccgcgtctggaacgttacaacggcctgccatccatggaaatcttaggccaggcggcaccgggtaaaagtaccggtgaagcaatggagctgatggaacaactggcgagcaaactgcctaccggtgttggctatgactggacggggatgtcctatcaggaacgtctctccggcaaccaggcaccttcactgtacgcgatttcgttgattgtcgtgttcctgtgtctggcggcgctgtacgagagctggtcgattccgttctccgttatgctggtcgttccgctgggggttatcggtgcgttgctggctgccaccttccgtggcctgaccaatgacgtttacttccaggtaggcctgctcacaaccattgggttgtcggcgaagaacgcgatccttatcgtcgaattcgccaaagacttgatggataaagaaggtaaaggtctgattgaagcgacgcttgatgcggtgcggatgcgtttacgtccgatcctgatgacctcgctggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 72 - DNA, 768 bp, Escherichia coli
  ssuB, NC_000913.3c994044-993277
atgaatactgctcgtctgaaccagggcacgccattgttgctcaatgcagtaagcaaacattacgcggaaaatatcgtcctgaaccaactggatttacatattccggcaggtcagtttgtggcggtggtgggccgcagcggtggtggcaaaagtaccctgctgcgcctgctggcaggtctggaaacgccaaccgcaggcgatgtgttagcgggcaccacaccgctggctgaaattcaggaagacacgcgaatgatgtttcaggatgcgcgtctgctaccgtggaaatcggtgattgataacgttgggttaggccttaaaggccagtggcgcgatgccgcacgtcgagcgttggctgcggtaggactggagaatcgcgcaggggaatggcctgccgcactttctggcgggcagaaacagcgagtggcgctggcaagagcgttgattcatcgaccgggattattgttgcttgatgaaccgctcggggcgctggacgccttaacgcgactcgagatgcaggatttgattgtgtcactttggcaggagcacggctttaccgtactgttggtgacgcatgatgtgagcgaagcggtagcgatggctgaccgggtgctgttaattgaagagggaaaaattggtctggatttgacagtggatatcccccgtccgcgtcgattagggtcggtgcgattagcggagctggaagcggaggtgttgcaacgggtgatgcaacgtggtgaatctgaaactcgcctgagaaaacaggggtaa

SEQ ID NO: 73 - DNA, 1539 bp, Escherichia coli
  emrB, NC_000913.32812616-2814154
atgcaacagcaaaaaccgctggaaggcgcgcaactggtcattatgacgattgcgctgtcactggcgacattcatgcaggtgctggactccaccattgctaacgtggcgatccccactatcgccgggaatctgggctcatcgctcagccagggaacgtgggtaatcacttctttcggggtggcgaatgccatctcgatcccgcttaccggctggctggcaaagcgcgtcggggaagtgaaactgttcctttggtccaccatcgcctttgctattgcgtcgtgggcgtgtggtgtctccagcagcctgaatatgctgatcttcttccgcgtgattcaggggattgtcgccgggccgttgatcccgctttcgcaaagtctattgctgaataactacccgccagccaaacgctcgatcgcgctggcgttgtggtcgatgacggtgattgtcgcgccaatttgcggcccgatcctcggcggttatatcagcgataattaccactggggctggatattcttcatcaacgtgccgattggcgtggcggtggtgttgatgacactgcaaactctgcgcggacgtgaaacccgcaccgaacggcggcggattgatgccgtggggctggcactgctggttattggtatcggcagcctgcagattatgctcgaccgcggtaaagagctggactggttttcatcacaggaaattatcatccttaccgtggtggcggtggtggctatctgcttcctgattgtctgggagctgaccgacgataacccgatagtcgatctgtcgttgtttaagtcgcgcaacttcaccatcggctgcttgtgtatcagcctcgcgtatatgctctacttcggcgctattgttctgctgccgcagttgttgcaggaggtctacggttacacggcgacctgggcaggtttggcctctgcgccggtagggattattccggtgatcctgtcgccgattatcggccgcttcgcgcataaactggatatgcggcggctggtaaccttcagctttattatgtatgccgtctgcttctactggcgtgcctatacctttgaaccaggtatggattttggcgcgtcggcctggccgcagtttatccaggggtttgcggtggcctgcttctttatgccgctgaccaccattacgctgtctggtttgccaccggaacgactggcggcggcatcgagcctctctaactttacgcgaacgctggcggggtctatcggcacgtcgataaccacgaccatgtggaccaaccgcgagtcgatgcaccatgcgcagttgactgagtcggtaaacccgttcaacccgaatgcccaggcgatgtacagtcaactggaagggcttgggatgacgcaacagcaggcatcaggctggattgcccagcagatcaccaatcaggggctgattatttccgccaatgagatcttctggatgtcagccgggatattcctcgtcctgctggggctggtgtggtttgctaaaccgccatttggcgcaggtggcggcggaggcggtgcgcactaa

SEQ ID NO: 74 - DNA, 1728 bp, Escherichia coli
  pstI, NC_000913.32534066-2535793
atgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaa